(19) United States
(12) Patent Application Publication
(10) Pub. No.: US 2024/0084348 A1
(43) Pub. Date: Mar. 14, 2024

(54) NUCLEIC ACID MUTAGENESIS METHODS

(71) Applicant: Editas Medicine, Inc., Cambridge, MA (US)

(72) Inventor: Barrett Ethan Steinberg, Somerville, MA (US)

(21) Appl. No.: 18/110,444

(22) Filed: Feb. 16, 2023

Related U.S. Application Data
(63) Continuation of application No. 16/498,994, filed on Sep. 27, 2019, now abandoned.
(60) Provisional application No. 62/478,257, filed on Mar. 29, 2017.

(51) Int. Cl. C12P 19/34; C12N 15/10; C40B 40/06; C40B 50/00

(52) U.S. Cl. CPC C12P 19/34; C12N 15/102; C40B 40/06; C40B 50/00

(57) ABSTRACT

The present disclosure relates to the manipulation of nucleic acids, and more particularly to systems and methods for nucleic acid mutagenesis.

### CROSS-REFERENCE TO RELATED APPLICATIONS

This application is a Continuation of U.S. application Ser. No. 16/498,994, filed Sep. 27, 2019, which is a national stage entry of PCT/US2018/025043, filed on Mar. 29, 2018, which claims the benefit of U.S. Provisional Application No. 62/478,257, filed Mar. 29, 2017, the contents each of which are hereby incorporated herein by reference in their entirety.

### FIELD OF THE INVENTION

The present disclosure relates to the manipulation of nucleic acids, and more particularly to systems and methods for nucleic acid mutagenesis.

### SEQUENCE LISTING

The present specification makes reference to a Sequence Listing (submitted electronically as an .XML file named “2011271-0241_SL.xml” on Nov. 17, 2023). The .XML file was generated on Nov. 14, 2023 and is 598,575 bytes in size. The entire contents of the Sequence Listing are herein incorporated by reference.

### BACKGROUND

Although a variety of nucleic acid mutagenesis methods are known in the art, current methods of comprehensively and/or randomly mutagenizing a polynucleotide of interest can be cumbersome and/or costly. In addition, mutagenesis methods such as error-prone polymerase chain reaction (e.g., using an error-prone DNA polymerase such as Mutazyme I or Mutazyme II DNA polymerase), while relatively fast only produce single base-pair mutations and often introduce biases (e.g., for or against particular codons or particular regions). The products of the reaction also need to be subcloned back into a cloning vector which adds steps and time to the process. Wildtype rates also tend to be higher, often near half of the library. Other mutagenesis methods (e.g., those that use PFunkel) require a single-stranded uracilated template, which is difficult and time-consuming to generate. They also require specialized, often proprietary enzymes (e.g., thermostable ligase and uracil-tolerant polymerase are necessary). Chemical synthesis of polynucleotide mutants is likewise costly and time-consuming (e.g., costing hundreds of thousands of dollars and taking months to prepare a library that can be prepared at a fraction of the cost and in a single day in accordance with the inventive methods described herein). Current methods (e.g., chemical synthesis, inverse PCR, cassette mutagenesis, etc.) also require separate reactions per mutant position which significantly increases the complexity of the process.

### SUMMARY

The present disclosure provides methods which can be used to comprehensively and/or randomly mutagenize a polynucleotide of interest. In some embodiments, the methods can achieve low levels of bias. In some embodiments, the methods can produce a library of mutated polynucleotides in a single day and in a single reaction tube. The methods have relative flexibility of polymerase choice and can be conveniently applied to various templates including a double-stranded polynucleotide template (e.g., a standard plasmid) rather than having to start from a particular type of template (e.g., a single-stranded uracilated template as discussed above). In some embodiments, the methods are able to produce libraries with low wildtype rates. In some embodiments, the methods can provide tunable mutation rates and/or targetable mutations.

In one aspect, the present disclosure provides a method of preparing mutant copies of a double-stranded template polynucleotide which comprises steps of: (a) providing a double-stranded template polynucleotide that comprises a first strand and a second strand, wherein the double-stranded template polynucleotide comprises a target region; (b) providing a pool of first oligonucleotide primers, wherein each of the first oligonucleotide primers is independently (i) capable of hybridizing to the first strand within the target region, and (ii) complementary to a sequence within the target region except for at least one mutagenic site where the first oligonucleotide primer and first strand are non-complementary; (c) providing a second oligonucleotide primer that is capable of hybridizing to the second strand; and (d) combining the double-stranded template polynucleotide, pool of first oligonucleotide primers, and second oligonucleotide primer under conditions that allow amplification of the double-stranded template polynucleotide, thereby generating mutant copies of the template polynucleotide, wherein each mutant copy includes a mutated version of at least a portion of the target region.

In some embodiments the double-stranded polynucleotide template may be provided in situ via an initial amplification step from a corresponding single-stranded polynucleotide template. In some embodiments, the double-stranded template polynucleotide is circular. In some embodiments, the double-stranded template polynucleotide is a plasmid.

In some embodiments, the target region encodes a polypeptide and comprises a plurality of codons, and the mutagenic site includes 1, 2 or all 3 nucleotides within one of the codons. In some embodiments, the pool of first oligonucleotide primers collectively span the entire length of the target region. In some embodiments, for each codon, there is at least one first oligonucleotide primer with a mutagenic site that includes 1, 2 or all 3 nucleotides within the codon. In some embodiments, each of the first oligonucleotide primers is present in the pool at approximately equimolar concentrations. In some embodiments, the first oligonucleotide primers is phosphorylated. In some embodiments, the second oligonucleotide primer is phosphorylated.

In some embodiments, the second oligonucleotide primer is capable of hybridizing to the second strand outside the target region and is not capable of hybridizing to the second strand in the target region.

In an additional aspect, the present disclosure provides methods which further comprise a step of providing a third oligonucleotide primer which is 100% complementary to the first strand of the double-stranded template polynucleotide, and wherein step (d) comprises combining the third oligonucleotide primer together with the double-stranded template polynucleotide, pool of first oligonucleotide primers, and second oligonucleotide primer under conditions that allow amplification of the double-stranded template polynucleotide.

In some embodiments, the third oligonucleotide is 100% complementary to the first strand of the double-stranded template polynucleotide outside of the target region. In some embodiments where the double-stranded template polynucleotide is circular, the third oligonucleotide primer is capable of hybridizing to a region immediately adjacent to the region to which the second oligonucleotide primer hybridizes.

In some embodiments, the conditions in step (d) comprise incubating the double-stranded template polynucleotide, the pool of first oligonucleotide primers, and the second oligonucleotide primer together in a reaction mixture. In some embodiments, the conditions in step (d) comprise incubating the double-stranded template polynucleotide, the pool of first oligonucleotide primers, the second oligonucleotide primer, and the third oligonucleotide primer together in a reaction mixture. The reaction mixture may comprise a DNA polymerase. The reaction mixture may comprise a DNA polymerase that has exonuclease activity (e.g., 3′ to 5′ exonuclease activity). In some embodiments, the DNA polymerase is not strand-displacing. In some embodiments, the DNA polymerase has an error rate of less than three base pair changes per kilobase of DNA (e.g., less than two base pair changes per kilobase of DNA or less than one base pair change per kilobase of DNA). In some embodiments, the DNA polymerase has an error rate that is at least 20-fold lower than that of Taq DNA polymerase under the same conditions (e.g., at least 40-fold lower or 50-fold lower).

In an additional aspect, the present disclosure provides methods which further comprise a step of: (e) combining the reaction mixture with one or more nucleases under conditions to allow activity of the one or more nucleases. In some embodiments, the one or more nucleases comprises a methylation-specific nuclease, an exonuclease, a nuclease specific for single-stranded DNA, or a combination thereof. In some embodiments, the one or more nucleases comprises a methylation-specific nuclease, an exonuclease, and a nuclease specific for single-stranded DNA. In some embodiments, the methylation-specific nuclease is DpnI. In some embodiments, the exonuclease cleaves in the 5′ to 3′ direction. In some embodiments, the exonuclease preferentially cleaves phosphorylated substrates (e.g., 5′ phosphorylated substrates). In some embodiments, the nuclease specific for single-stranded DNA is Exonuclease I (ExoI).

In some embodiments, the molar ratio of the pool of first oligonucleotide primers to the template polynucleotide is 1:1 or greater. In some embodiments, the molar ratio of the pool of first oligonucleotide primers to the template polynucleotide is 50:1 or greater. In some embodiments, the molar ratio of the pool of first oligonucleotide primers to the template polynucleotide is 100:1 or greater. In some embodiments, the molar ratio of the second oligonucleotide primer to the template polynucleotide is 1:1 or greater. In some embodiments, the molar ratio of the second oligonucleotide primer to the template polynucleotide is 50:1 or greater. In some embodiments, the molar ratio of the second oligonucleotide primer to the template polynucleotide is 100:1 or greater.

In an additional aspect, the present disclosure provides methods which further comprise a step of transforming the library of mutated polynucleotides into cells.

In an additional aspect, the present disclosure provides a library obtained by any of the methods described herein.

### BRIEF DESCRIPTION OF THE DRAWING

FIG. 1 depicts an overview of an embodiment of the methods of the present invention. Pooled degenerate, mutagenic oligonucleotides are added to a circular double-stranded template DNA (shown as solid arrows above the circular double-stranded template DNA) along with a non-degenerate, non-mutagenic reverse primer (solid arrow) and an optional non-degenerate, non-mutagenic forward primer (dotted arrow). Exemplary reaction products are shown in the bottom part of FIG. 1. Multiple amplification cycles may recombine incomplete products into nicked circular DNA. Linear products may be phosphorylated in embodiments in which phosphorylated primers are used.

FIGS. 2A and 2B provide schematic representations of the domain organization of S. pyogenes Cas9.

FIG. 2A shows the organization of the Cas9 domains, including amino acid positions, in reference to the two lobes of Cas9 (recognition (REC) and nuclease (NUC) lobes).

FIG. 2B shows the percent homology of each domain across 83 Cas9 orthologs.

FIGS. 3A-3G depict an alignment of Cas9 sequences (Chylinski 2013). The N-terminal RuvC motif is boxed and indicated with a “Y.” The other two RuvC motifs are boxed and indicated with a “B.” The HNH domain is boxed and indicated by a “G.” Sm: S. mutans (SEQ ID NO:1); Sp: S. pyogenes (SEQ ID NO:2); St: S. thermophilus (SEQ ID NO:4); and Li: L. innocua (SEQ ID NO:5). “Motif” (SEQ ID NO:14) is a consensus sequence based on the four sequences. Residues conserved in all four sequences are indicated by single letter amino acid abbreviation; “*” indicates any amino acid found in the corresponding position of any of the four sequences; and “-” indicates absent.

FIGS. 4A-4B show an alignment of the N-terminal RuvC motif from the Cas9 molecules disclosed in Chylinski 2013 with sequence outliers removed (SEQ ID NOs:52-95, 120-123). The last line of FIG. 4B identifies 4 highly conserved residues.

FIGS. 5A-5B show an alignment of the N-terminal RuvC motif from the Cas9 molecules disclosed in Chylinski 2013 (SEQ ID NOs:52-123). The last line of FIG. 5B identifies 3 highly conserved residues.

FIGS. 6A-6C show an alignment of the HNH domain from the Cas9 molecules disclosed in Chylinski 2013 (SEQ ID NOs:124-198). The last line of FIG. 6C identifies conserved residues.

FIGS. 7A-7B show an alignment of the HNH domain from the Cas9 molecules disclosed in Chylinski 2013 with sequence outliers removed (SEQ ID NOs:124-141, 148, 149, 151-153, 162, 163, 166-174, 177-187, 194-198). The last line of FIG. 7B identifies 3 highly conserved residues.

FIGS. 8A-8I are representations of several exemplary gRNAs.

FIG. 8A depicts a modular gRNA molecule derived in part (or modeled on a sequence in part) from Streptococcus pyogenes (S. pyogenes) as a duplexed structure (SEQ ID NOs:39 and 40, respectively, in order of appearance);

FIG. 8B depicts a unimolecular gRNA molecule derived in part from S. pyogenes as a duplexed structure (SEQ ID NO:41);

FIG. 8C depicts a unimolecular gRNA molecule derived in part from S. pyogenes as a duplexed structure (SEQ ID NO:42);

FIG. 8D depicts a unimolecular gRNA molecule derived in part from S. pyogenes as a duplexed structure (SEQ ID NO:43);

FIG. 8E depicts a unimolecular gRNA molecule derived in part from S. pyogenes as a duplexed structure (SEQ ID NO:44);

FIG. 8F depicts a modular gRNA molecule derived in part from Streptococcus thermophilus (S. thermophilus) as a duplexed structure (SEQ ID NOs:45 and 46, respectively, in order of appearance);

FIG. 8G depicts an alignment of modular gRNA molecules of S. pyogenes and S. thermophilus (SEQ ID NOs:39, 45, 47, and 46, respectively, in order of appearance).

FIGS. 8H-8I depicts additional exemplary structures of unimolecular gRNA molecules.

FIG. 8H shows an exemplary structure of a unimolecular gRNA molecule derived in part from S. pyogenes as a duplexed structure (SEQ ID NO:42).

FIG. 8I shows an exemplary structure of a unimolecular gRNA molecule derived in part from S. aureus as a duplexed structure (SEQ ID NO:38).

FIG. 9 illustrates gRNA domain nomenclature using an exemplary gRNA sequence (SEQ ID NO:42).

FIGS. 10A-10B depict the codon mutation rate of Cas9 libraries prepared as described in Example 1.

FIG. 10A depicts the codon mutation rate for a Cas9 library that was prepared in the presence of a non-degenerate, non-mutagenic forward primer. The average mutation rate observed was 5.1 codon mutations per mutated polynucleotide (with a median of 3 and a standard deviation of 8.7). The wildtype percentage was 8.7%.

FIG. 10B depicts the codon mutation rate for a Cas9 library that was prepared without the non-degenerate, non-mutagenic forward primer. The average mutation rate was 5.5 codon mutations per mutated polynucleotide (with a median of 5 and a standard deviation of 4.9). The wildtype percentage was 2.6%.

FIG. 11 depicts the number of mutations found for every codon position in a Cas9 library prepared as described in Example 1. A linear fit of the results was (y=−0.116x with R 2=0.2 where x is codon position and y is frequency of mutations). This demonstrates a weak and slight negative correlation (i.e., low bias).

FIG. 12 depicts the degree of codon bias based on the number of amino acid mutations found for every codon position in a Cas9 library prepared as described in Example 1.

FIG. 13 depicts the degree of amino acid bias based on the normalized amino acid representation across all amino acids in a Cas9 library prepared as described in Example 1. Counts of amino acid mutations were normalized to the expected random distribution of amino acid mutations due to codon bias given a random (NNN) codon.

FIGS. 14A-14B depict an exemplary codon optimized nucleic acid sequence encoding a Cas9 molecule of S. pyogenes (SEQ ID NO:3).

FIGS. 15A-15B depicts an exemplary codon optimized nucleic acid sequences encoding a Cas9 molecule of S. aureus (SEQ ID NO:7).

FIGS. 16A-16B depicts an exemplary codon optimized nucleic acid sequences encoding a Cas9 molecule of S. aureus (SEQ ID NO:8).

FIGS. 17A-17B depicts an exemplary codon optimized nucleic acid sequences encoding a Cas9 molecule of S. aureus (SEQ ID NO:9).

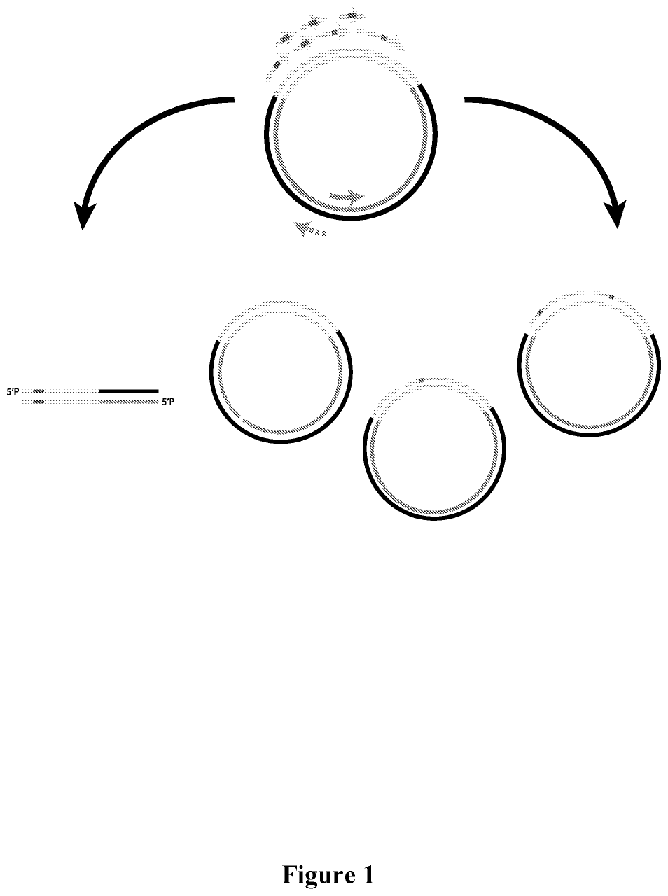

### DEFINITIONS

Throughout the specification, several terms are employed that are defined in the following paragraphs. Other definitions are also found within the body of the specification.

As used herein, the terms “about” and “approximately,” in reference to a number, is used herein to include numbers that fall within a range of 20%, 10%, 5%, or 1% in either direction (greater than or less than) of the number unless otherwise stated or otherwise evident from the context (except where such number would exceed 100% of a possible value).

As used herein, the term “amplification,” when used in reference to polynucleotides, refers to a method that increases the representation in a population of a specific nucleotide sequence (e.g., from a template polynucleotide) in a sample by producing multiple (i.e., at least 2) copies of the desired nucleotide sequence. Methods for nucleic acid amplification are known in the art and include, but are not limited to, polymerase chain reaction (PCR) and ligase chain reaction (LCR). Variants of standard PCR or LCR reactions can also be used. A “copy” or “amplicon” does not necessarily have perfect sequence complementarity or identity to the nucleotide sequence in the template polynucleotide. Unless otherwise specified, one or more copies can comprise one or more mutant copies, i.e., copies containing one or more mutations (“mutant copies”) as compared to the nucleotide sequence in the template polynucleotide. Mutant copies can comprise mutations in one or more bases. For example, for template polynucleotides that comprise a coding region with a plurality of codons, mutant copies can comprise mutations in one or more than one codon and within each codon, there can be mutations in one, two, or all three nucleotides of the codon. In general, “mutations” will be understood to include substitutions, insertions or deletions relative to the template polynucleotide.

As used herein, the term “complementary” refers to nucleotides or nucleotide sequences that base-pair according to the standard Watson-Crick complementary rules (adenine “A” base pairs with thymine “T”, and guanine “G” base pairs with cytosine “C”). Nucleotide sequences that are “100% complementary” or which exhibit “100% complementarity” are nucleotide sequences which base-pair with one another across the entirety of at least one of the two nucleotide sequences. An oligonucleotide can be “100% complementary” to a template polynucleotide that is longer than the oligonucleotide (i.e., the oligonucleotide is “100% complementary” to the template polynucleotide if the entire sequence of the oligonucleotide base-pairs with a portion of the template polynucleotide).

As used herein, the phrase “corresponding to,” when used to describe positions or sites within nucleotide sequences, is used herein as it is understood in the art. As is well known in the art, two or more nucleotide sequences can be aligned using standard bioinformatic tools, including, but not limited to, programs such as BLAST, ClustalX, Sequencher, etc. Even though the two or more nucleotide sequences may not match exactly and/or do not have the same length, an alignment of the nucleotide sequences can still be performed and, if desirable, a “consensus” sequence generated. Indeed, programs and algorithms used for alignments typically tolerate definable levels of differences, including insertions, deletions, inversions, polymorphisms, point mutations, etc. Such alignments can aid in the determination of which positions in one nucleotide sequence correspond to which positions in other nucleotide sequences. When used in the context of nucleotide sequences, the term “corresponding position” refers to a position in one sequence that corresponds to a particular position in another sequence.

As used herein, the term “degenerate” when used to refer to an oligonucleotide or nucleotide sequence, refers to the characteristic that the oligonucleotide or nucleotide sequence is actually a mixture in which one or more positions contain two or more different bases.

As used herein, the term “error rate,” when used in reference to a DNA polymerase or a similar enzyme, refers to the probability of the DNA polymerase or similar enzyme introducing one base pair mutation per cycle of amplification. The error rate may be lower in a DNA polymerase or similar enzyme with proof-reading ability.

As used herein, the term “hybridize” or “hybridization” refers to a process where two strands in a double-stranded polynucleotide anneal to each other under appropriately stringent conditions. The phrase “is capable is hybridizing to” refers to the ability of two nucleotide sequences to hybridize to each other under typical hybridization conditions (e.g., in the context of a typical amplification reaction, “hybridize” would refer to the interaction of two complementary nucleotide sequences during the annealing phase). As understood by one of ordinary skill in the art, nucleotide sequences need not have perfect sequence complementarity to hybridize with one another. Those skilled in the art understand how to estimate and adjust the stringency of hybridization conditions such that sequences having at least a desired level of complementary will stably hybridize, while those having lower complementary will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, et al. 1994, Current Protocols in Molecular Biology. John Wiley & Sons, Secaucus, N.J.

As used herein, the term “non-complementary” refers to nucleotides or nucleotide sequences that do not base-pair according to the standard Watson-Crick complementary rules.

As used herein, the term “mutant copy,” in reference to a template polynucleotide, refers to a copy of at least a portion of a template polynucleotide that contains one or more mutations. The mutant copies can be generated, for example, using nucleic acid amplification protocols as discussed herein. Mutant copies can comprise mutations in one or more nucleotides. For example, for polynucleotides that comprise a coding region with a plurality of codons, mutant copies can comprise mutations in one or more than one codon and within each codon, there can be mutations in one, two, or all three nucleotides of the codon. A mutant copy of a double-stranded template polynucleotide can include a copy of all or just a portion of one strand of the template polynucleotide. A mutant copy of a double-stranded template polynucleotide can also include a copy of all or just a portion of both strands of the template polynucleotide.

As used herein, the terms “nucleic acid”, “nucleic acid molecule” or “polynucleotide” are used herein interchangeably. They refer to a polymer of deoxyribonucleotides or ribonucleotides in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products. DNAs and RNAs are both polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

As used herein, the term “oligonucleotide” refers to a string of nucleotides. Oligonucleotides may be obtained by a number of methods including, for example, chemical synthesis, restriction enzyme digestion or PCR. As will be appreciated by one skilled in the art, the length of an oligonucleotide (i.e., the number of nucleotides) can vary widely, often depending on the intended function or use of the oligonucleotide. Generally, oligonucleotides comprise between about 5 and about 300 nucleotides, for example, between about 15 and about 200 nucleotides, between about 15 and about 100 nucleotides, between about 15 and about 50 nucleotides, and between about 20 and about 40 nucleotides. In some embodiments, oligonucleotides are between about 20 and about 40 nucleotides in length.

As used herein, the term “plurality” means more than one.

As used herein, the term “polypeptide” generally has its art-recognized meaning of a polymer of amino acids. The term is also used to refer to specific functional classes of polypeptides, such as, for example, nucleases, antibodies, etc.

As used herein, the term “primer” is interchangeable with “oligonucleotide primer” and is used herein to refer to an oligonucleotide that acts as a point of initiation of synthesis of a primer extension product when hybridized to a template polynucleotide, when placed under suitable conditions (e.g., buffer, salt, temperature and pH), in the presence of nucleotides and an agent for nucleic acid polymerization (e.g., a DNA-dependent or RNA-dependent polymerase). The primer is preferably single-stranded for maximum efficiency in amplification, but may alternatively be double-stranded. If double-stranded, the primer may first be treated (e.g., denatured) to allow separation of its strands before being used to prepare extension products. Such a denaturation step is typically performed using heat, but may alternatively be carried out using alkali, followed by neutralization. A typical primer comprises a sequence of about 10 to about 50, e.g., about 20 to about 40 nucleotides that is complementary to a sequence in a template polynucleotide.

### DETAILED DESCRIPTION OF CERTAIN EMBODIMENTS

The present invention encompasses the recognition that random mutagenesis methods that introduce little or no bias (e.g., for or against particular sub-regions and/or for or against particular codons), particularly such methods that are cost-effective and can be done quickly, are desired.

### Methods of Preparing a Library of Mutated Polynucleotides

In one aspect, the present disclosure provides methods of preparing a library of mutated polynucleotides. In one embodiment, the methods are for preparing mutant copies of a double-stranded template polynucleotide. In some embodiments, provided methods allow comprehensive mutagenesis over an entire nucleotide sequence of interest. To the inventors' knowledge, provided methods are faster and more cost-effective than existing methods of comprehensively mutating a nucleotide sequence of interest. In some embodiments, comprehensive mutagenesis using methods of the present invention is accomplished in a single day, and/or using a single reaction tube.

In certain embodiments, provided are methods which start with a double-stranded template polynucleotide that comprises a first strand and a second strand. The double-stranded template polynucleotide comprises a target region which is intended to be mutated. The target region can be one contiguous nucleotide sequence or comprised of at least two non-contiguous nucleotides or nucleotide sequences (e.g., a codon or series of codons) that are intended to be mutated separated by a nucleotide or nucleotide sequence (e.g., a codon or series of codons) that is not intended to be mutated. A pool of first oligonucleotide primers is provided which are each capable of hybridizing to the first strand within the target region. It is to be understood that, as used herein, the terms “capable of hybridizing” (or “not capable of hybridizing”) mean that the oligonucleotide in questions is (or is not) capable of hybridizing to the corresponding polynucleotide under a condition of the claimed method. Each of the first oligonucleotide primers is independently complementary to a sequence within the target region except for at least one mutagenic site where the first oligonucleotide primer and first strand are non-complementary (i.e., the first oligonucleotide primers each hybridize to independent sequences within the target region and the mutagenic site(s) for each first oligonucleotide primer are independent of the mutagenic site(s) of other first oligonucleotide primers in the pool). In some embodiments, a mutagenic site for a particular first oligonucleotide primer consists of no more than three nucleotides. In some embodiments, the mutagenic site consists of three nucleotides (e.g., a codon in a coding region of a template polynucleotide). In some embodiments, the mutagenic site consists of two nucleotides. In some embodiments, the mutagenic site consists of one nucleotide. In some embodiments the mutation introduced by a first oligonucleotide primer is a substitution. In some embodiments the mutation introduced by a first oligonucleotide primer is a deletion. In some embodiments the mutation introduced by a first oligonucleotide primer is an insertion. A second oligonucleotide primer is provided which is capable of hybridizing to the second strand of the double-stranded template polynucleotide. The double-stranded template polynucleotide, pool of first oligonucleotide primers, and second oligonucleotide primer are then combined under conditions that allow amplification of the double-stranded template polynucleotide. The methods generate mutant copies of the template polynucleotide, wherein each mutant copy includes a mutated version of at least a portion of the target region.

### Target Region

Methods of the present invention are generally applicable to any polynucleotide sequence that one desires to mutagenize and/or for which one desires to generate a library of mutants. Such a polynucleotide sequence is referred to herein as the “target region.” Any type of polynucleotide sequence can serve as a target region, including, but not limited to, gene elements that are not transcribed, sequences that serves a templates for non-coding RNAs, sequences that encode a polypeptide, any combination of the aforementioned sequences or gene elements, and any fragment of the aforementioned sequences or gene elements, etc.

In some embodiments, the target region is a gene regulatory element (e.g., a DNA binding site, a promoter, an enhancer, etc.) or a fragment thereof. In some embodiments, the target region is or comprises a DNA aptamer or a fragment thereof.

In some embodiments, the target region serves as a template for all or portion of a non-coding RNA, i.e., an RNA molecule that is not translated into a polypeptide. Such a non-coding RNA may or may not be functional and may or may not be processed after being transcribed. Non-limiting examples of non-coding RNAs include RNA aptamers, guide RNAs (gRNAs), ribozymes, microRNAs (miRNAs), small interfering RNAs (siRNAs), long non-coding RNAs (lncRNAs), piwi RNAs (piRNAs), transfer RNAs (tRNAs), ribosomal RNAs (rRNAs), small nucleolar RNAs (snoRNAs), small nuclear RNAs (snRNAs), extracellular RNAs (exRNAs), and small Cajal body RNAs (scaRNAs).

In some embodiments, the target region encodes all or a portion of a polypeptide, e.g., the target region comprises a coding region with a plurality of codons. Methods of the present invention are generally applicable to any type of polypeptide, non-limiting examples of which include antibodies and antibody-binding fragments thereof, structural proteins (e.g., keratin, elastin, and collagen), transport proteins (e.g., hemoglobin), DNA-regulatory proteins, DNA-binding proteins, DNA-structural proteins (e.g., histones), enzymes, nutrient storage proteins (e.g., ferritin, ovalbumin, and casein), protein hormones, receptor proteins, contractile proteins (e.g., actin and myosin), and any fragment and/or fusion thereof.

In some embodiments, the target region encodes all or a portion of an antibody or antigen-binding fragment thereof. For example, in some embodiments, the template polynucleotide encodes an antibody, the target region encodes the variable region of the antibody and the methods are used to generate a library of mutated polynucleotides that encode new versions of the antibody with mutated complementarity determining regions (CDR). In another example, the template polynucleotide encodes an antibody, the target region encodes the variable region of the antibody and the methods are used to generate a library of mutated polynucleotides that encode new versions of the antibody with mutated framework regions (but otherwise original CDRs).

In some embodiments, the target region encodes all or a portion of an enzyme. In some embodiments, the enzyme is an oxidoreductase. In some embodiments, the enzyme is a hydrolase. In some embodiments, the enzyme is a transferase. In some embodiments, the enzyme is a lyase. In some embodiments, the enzyme is an isomerase. In some embodiments, the enzyme is a ligase. For example, in some embodiments, the template polynucleotide encodes an enzyme, the target region encodes a functional domain of the enzyme and the methods are used to generate a library of mutated polynucleotides that encode new versions of the enzyme with a mutated active site. In another example, the template polynucleotide encodes an enzyme, the target region encodes a functional domain of the enzyme and the methods are used to generate a library of mutated polynucleotides that encode new versions of the enzyme (but an otherwise original active site).

As a non-limiting example where the enzyme is a hydrolase, the target region can encode all or a portion of a nuclease. In some embodiments, the nuclease is site-specific (e.g., a restriction endonuclease, a meganuclease, a TALEN, a zinc finger nuclease, etc.). In some embodiments, the site specificity of a site-specific nuclease is conferred by an accessory molecule, which may or may not be encoded by another element present in a double-stranded template polynucleotide. For example, the CRISPR-associated (Cas) nucleases are guided to specific sites by “guide RNAs” or gRNAs as described herein. In some embodiments, the target region encodes all or a portion of a CRISPR-associated (Cas) nuclease, a fragment thereof, or a variant thereof (e.g., a catalytically inactive variant or a “nickase” variant which has been mutated in such a way that it only cleaves a single strand of double-stranded DNA). In some embodiments, the target region encodes all or a portion of a Cas nuclease, and a nucleotide sequence that serves as a template for a gRNA suitable for guiding the Cas nuclease is also included in the double-stranded template polynucleotide. In some such embodiments, the nucleotide sequence that serves as a template for the gRNA is also part of the target region. In some such embodiments, the nucleotide sequence that serves as a template for the gRNA is not part of the target region. Other exemplary embodiments of the present invention that involve target regions that encode all or portions of Cas nucleases are discussed in more detail below.

In some embodiments, the target region comprises at least 300 nucleotides, at least 400 nucleotides, at least 500 nucleotides, at least 600 nucleotides, at least 700 nucleotides, at least 800 nucleotides, at least 900 nucleotides, at least 1000 nucleotides, at least 1100 nucleotides, at least 1200 nucleotides, at least 1300 nucleotides, at least 1400 nucleotides, at least 1500 nucleotides, at least 1600 nucleotides, at least 1700 nucleotides, at least 1800 nucleotides, at least 1900 nucleotides, at least 2000 nucleotides, at least 2100 nucleotides, at least 2200 nucleotides, at least 2300 nucleotides, at least 2400 nucleotides, at least 2500 nucleotides, at least 2600 nucleotides, at least 2700 nucleotides, at least 2800 nucleotides, at least 2900 nucleotides, or at least 3000 nucleotides.

In some embodiments, the target region encodes all or a portion of a polypeptide and comprises a plurality of codons, and each mutagenic site includes 1, 2, or all 3 nucleotides within one of the codons. In some embodiments, the target region comprises at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 codons. In some embodiments, provided methods are used to mutate a portion of a polypeptide (i.e., the template polynucleotide encodes a polypeptide but the target region encodes only a portion of the polypeptide). In some such embodiments, the target region comprises fewer than 100, fewer than 150, fewer than 200, fewer than 250, fewer than 300, fewer than 350, fewer than 400, fewer than 450, or fewer than 500 codons.

In some embodiments, provided methods can be used to target each codon in the target region, that is, provided methods allow the generation of libraries of mutated polynucleotides that collectively contain at least one mutation (relative to the target region in the template polynucleotide) in each codon of the target region. For example, in some embodiments, for each codon, there is at least one first oligonucleotide primer in the pool with a mutagenic site that includes 1, 2 or all 3 nucleotides within the codon.

For example, in certain embodiments, provided are methods comprising steps of: (a) providing a double-stranded template polynucleotide that comprises a first strand and second strand, wherein the double-stranded template polynucleotide comprises a target region that encodes all or a portion of a polypeptide and comprises a plurality of codons; (b) providing a pool of first oligonucleotide primers, wherein each of the first oligonucleotide primers is independently: (i) capable of hybridizing to the first strand within the target region, (ii) complementary to a sequence within the target region except for at least one mutagenic site where the first oligonucleotide primer and first strand are non-complementary, wherein the mutagenic site includes 1, 2 or all 3 nucleotides within one of the codons; (c) providing a second oligonucleotide primer that is capable of hybridizing to the second strand; and (d) combining the double-stranded template polynucleotide, pool of first oligonucleotide primers, and second oligonucleotide primer under conditions that allow amplification of the double-stranded template polynucleotide, thereby generating mutant copies of the template polynucleotide, wherein each mutant copy includes a mutated version of at least a portion of the target region

### Mutagenesis Coverage

As noted previously, the target region can be one contiguous nucleotide sequence or comprised of at least two non-contiguous nucleotides or nucleotide sequences (e.g., a codon or series of codons) that are intended to be mutated separated by a nucleotide or nucleotide sequence (e.g., a codon or series of codons) that is not intended to be mutated.

Thus, in some embodiments, the pool of first oligonucleotide primers collectively span the entire length of the target region (i.e., they collectively cover a set of mutagenic sites that include each nucleotide position within the target region). In other embodiments, the pool of first oligonucleotide primers does not collectively span the entire length of the target region (i.e., the pool lacks first oligonucleotide primers for at least some nucleotide positions of the target region that are not intended to be mutated). For example, in some embodiments, the pool of first oligonucleotide primers collectively spans one or more regions of interest within the target region. Alternatively or additionally, the mutagenic site or sites within the target region may each be specific pre-determined nucleotide position or positions that collectively comprise a smaller subset of all the possible nucleotide positions in the target region. For example, in some embodiments, combinations of one or more specific mutations of interest (e.g., that have been identified previously) can be generated using such pools of first oligonucleotide primers with methods disclosed herein.

In some embodiments, the largest stretch of the target region that is not mutated by the methods of the present invention is no greater than 10%, 9%, 8%, 7%, 6%, 5%, 4%, 3%, 2%, or 1% of the length of the target region. In some embodiments, the largest stretch is no greater than 5% of the length of the target region. In some embodiments, the largest stretch is no greater than 3% of the length of the target region. In some embodiments, the largest stretch is no greater than 1% of the length of the target region.

In some embodiments, the stretch(es) of the target region that are not mutated by the methods of the present invention are collectively no greater than 50%, 25%, 10%, 9%, 8%, 7%, 6%, 5%, 4%, 3%, 2%, or 1% of the length of the target region. In some embodiments, they are collectively no greater than 5% of the length of the target region. In some embodiments, they are collectively no greater than 3% of the length of the target region. In some embodiments, they are collectively no greater than 1% of the length of the target region.

In some embodiments, the largest stretch of the target region that is not mutated by the methods of the present invention is no greater than 50, 25, 10, 9, 8, 7, 6, 5, 4, 3, 2, or 1 nucleotide(s) in length. In some embodiments, the largest stretch is no greater than 5 nucleotide(s) in length. In some embodiments, the largest stretch is no greater than 3 nucleotide(s) in length. In some embodiments, the largest stretch is no greater than 1 nucleotide in length.

In some embodiments, the stretch(es) of the target region that are not mutated by the methods of the present invention are collectively no greater than 500, 250, 100, 50, 10, 9, 8, 7, 6, 5, 4, 3, 2, or 1 nucleotide(s) in length. In some embodiments, they are collectively no greater than 5 nucleotide(s) in length. In some embodiments, they are collectively no greater than 3 nucleotide(s) in length. In some embodiments, they are collectively no greater than 1 nucleotide in length.

In some embodiments in which the target region comprises a plurality of codons, the largest stretch of the target region that is not mutated by the methods of the present invention is no greater than 10%, 9%, 8%, 7%, 6%, 5%, 4%, 3%, 2%, or 1% of the plurality of codons. In some embodiments, the largest stretch is no greater than 5% of the plurality of codons. In some embodiments, the largest stretch is no greater than 3% of the plurality of codons. In some embodiments, the largest stretch is no greater than 1% of the plurality of codons.

In some embodiments in which the target region comprises a plurality of codons, the stretch(es) of the target region that are not mutated by the methods of the present invention are collectively no greater than 50%, 25%, 10%, 9%, 8%, 7%, 6%, 5%, 4%, 3%, 2%, or 1% of the plurality of codons. In some embodiments, they are collectively no greater than 5% of the plurality of codons. In some embodiments, they are collectively no greater than 3% of the plurality of codons. In some embodiments, they are collectively no greater than 1% of the plurality of codons.

In some embodiments, every codon in the target region is mutated. In some embodiments, every nucleotide position in the target region is mutated.

In some embodiments, the library of mutated polynucleotides that is generated collectively comprises at least one mutation in each codon. In some embodiments, the library of mutated polynucleotides that is generated collectively comprises at least one mutation at every nucleotide position in the target region.

### Double-Stranded Template Polynucleotides

In some embodiments, the double-stranded template polynucleotide is circular. For example, the template polynucleotide can be a plasmid. In some such embodiments, provided methods further comprise, after the library of mutated polynucleotides is generated, one or more steps to remove non-circular polynucleotides, as described herein.

In some embodiments, the double-stranded template polynucleotide further comprises, in addition to the target region, one or more of the following elements: one or more origins of replication, one or more antibiotic resistance genes, one or more regulatory elements (such as, for example, a promoter that drives expression of one or more genes), and one or more selectable markers distinct from the one or more antibiotic resistance genes.

In some embodiments in which the target region encodes a polypeptide, the template polynucleotide further comprises an element that encodes an accessory molecule that may be used together with the polypeptide. For example, the polypeptide may function together with the accessory molecule.

In some embodiments, the accessory molecule is an RNA molecule. For example, the accessory molecule can be a gRNA that is used together with a CRISPR-associated (Cas) nuclease as discussed further herein.

In some embodiments, the accessory molecule is a polypeptide.

### Pool of First Oligonucleotide Primers

In certain embodiments, inventive methods comprise a step of providing a pool of first oligonucleotide primers, wherein each of the first oligonucleotide primers in the pool is independently capable of hybridizing to the first strand of the double-stranded template polynucleotide and wherein each of the first oligonucleotide primers is complementary to a sequence within the target region except for at least one mutagenic site where the first oligonucleotide primer and first strand are non-complementary.

In some embodiments, each mutagenic site for each first oligonucleotide primer corresponds to an internal position or positions within the first oligonucleotide primer. That is, the mutagenic site does not correspond to a position at either end (5′ or 3′) of the first oligonucleotide primer.

In some embodiments, each of the first oligonucleotide primers is present in the pool at approximately equimolar concentrations. For example, in embodiments in which the target region comprises a plurality of codons, mutagenesis of all codons with low bias or no bias for or against any particular codon(s) can be facilitated by using approximately equimolar concentrations of each first oligonucleotide primer.

In some embodiments, each of the first oligonucleotide primers is present in the pool at concentrations that are not approximately equimolar. For example, in embodiments in which the target region comprises a plurality of codons, mutagenesis of one or more particular codons can be biased for or against by using higher or lower, respectively, concentrations of first oligonucleotide primers with corresponding mutagenic sites in the particular codons.

In some embodiments, the sequences of each of the first oligonucleotide primers is designed to optimize primer binding.

In some embodiments, the sequences of each of the first oligonucleotide primers is designed to equalize primer binding.

For example, sequences for first oligonucleotide primers can be generated through a computer script or algorithm designed to generate sequences of oligonucleotide primers with certain desired characteristics. For example, in some embodiments, the first oligonucleotide primers are designed so that they do not form hairpins and/or do not dimerize with other first oligonucleotide primers in the pool. In some embodiments, the first oligonucleotide primers are designed to have Tm values which are within a particular range of each other (e.g., within 5° C., within 4° C., within 3° C., within 2° C. or within 1° C. of each other). In some embodiments, first oligonucleotide primers are designed to have a GC % within a particular range. In some embodiments, the first oligonucleotide primers are designed to avoid having any secondary structure (e.g., to avoid any hairpins). In some embodiments, the first oligonucleotide primers are designed to avoid repeats of 2 or more of the same nucleotide, for example 3 or more of the same nucleotide, for example 4 or more of the same nucleotide. In some embodiments, the first oligonucleotide primers are designed to provide end stability.

In some embodiments, the pool of first oligonucleotide primers is subjected to conditions to allow phosphorylation of oligonucleotide primers. For example, the pool of first oligonucleotide primers can be treated with a kinase. In some embodiments, the kinase is a kinase that phosphorylates DNA substrates at their 5′ ends.

In some embodiments, each of the first oligonucleotide primers is phosphorylated. In some embodiments, each of the first oligonucleotide primers is phosphorylated at its 5′ end.

In some embodiments, each of the first oligonucleotide primers is detectably labeled, e.g., at its 5′ end.

In some embodiments, the target region comprises a plurality of codons and each first oligonucleotide primer is capable of hybridizing to a sequence within the target region. In some embodiments, the first strand is the coding strand, also known in the art as the “sense” strand. In some embodiments, the first strand is the non-coding strand, also known in the art as the “anti-sense” strand.

In certain embodiments in which the target region comprises a plurality of codons, each of the first oligonucleotide primers is complementary to a sequence within the target region except for a mutagenic site within at least one “mutagenic codon.”

Instead of a complementary sequence to the mutagenic codon(s), in some embodiments, the first oligonucleotide primer contains a sequence that is non-complementary to the mutagenic codon(s), each of which generally comprises three nucleotides. By “non-complementary” it is meant that at least one of the nucleotides in the part of the first oligonucleotide primer that corresponds to the mutagenic codon is not complementary to the nucleotide in the mutagenic codon at the corresponding position. Thus, in some embodiments, the first oligonucleotide primer contains one, two, or three nucleotides that are not complementary to the nucleotide at the corresponding position, and such non-complementary nucleotides correspond to a position within the mutagenic codon.

In some embodiments, this non-complementary sequence contains the same number of nucleotides as that of corresponding nucleotides within the mutagenic codon so as to not interfere with translation.

For example, if the mutagenic codon has the sequence 5′-ACG-3′ in the first strand, the corresponding complementary sequence would be 5′-CGT-3′. However, for at least some of the first oligonucleotide primer(s) for which the mutagenic codon has the sequence 5′-ACG-3′, instead of having 5′-CGT-3′ at the corresponding location, the first oligonucleotide primer could have any other trinucleotide instead at that location, for example, any of the listed sequences in Table 1 (where sequences are presented in the 5′ to 3′ direction)

TABLE 1
Containing one mutation with respect to the
complementary sequence CGT

| | | |
|---|---|---|
| AGT | CAT | CGA |
| GGT | CCT | CGC |
| TGT | CTT | CGG |

Containing two mutations with respect to the
complementary sequence CGT

| | | |
|---|---|---|
| AAT | GAT | TAT |
| ACT | GCT | TCT |
| ATT | GTT | TTT |
| AGA | GGA | TGA |
| AGC | GGC | TGC |
| AGG | GGG | TGG |
| CAA | CCA | CTA |
| CAC | CCC | CTC |
| CAG | CCG | CTG |

Containing three mutations with respect to
the complementary sequence CGT

| | | |
|---|---|---|
| AAA | GAA | TAA |
| AAC | GAC | TAC |
| AAG | GAG | TAG |
| ACA | GCA | TCA |
| ACC | GCC | TCC |
| ACG | GCG | TCG |
| ATA | GTA | TTA |
| ATC | GTC | TTC |
| ATG | GTG | TTG |

In some embodiments, the target region encodes a polypeptide. In some such embodiments, each of the first oligonucleotide primers can, instead of having the complementary sequence to its mutagenic codon, have any other trinucleotide at that location, except for a trinucleotide that would encode a stop codon or otherwise interfere with translation of the polypeptide.

For example, according to the genetic code that has been identified as universal across all organisms studied thus far, 5′-UAA-3′, 5′-UAG-3′, and 5′-UGA-3′ are translation stop codons. According to this genetic code, in embodiments in which the first strand is the coding strand, the first oligonucleotide primers would correspond to the non-coding strand, and the sequences 5′-TTA-3′, 5′-CTA-3′, and 5′-TCA-3′ in the first oligonucleotide primers, would ultimately be replicated as stop codons. In some embodiments, therefore, the first oligonucleotide primers, instead of having a complementary sequence to the mutagenic codon, could have any other non-complementary sequence except for 5′-TTA-3′, 5′-CTA-3′, and 5′-TCA-3′.

According to the universal genetic code, in embodiments in which the first strand is the non-coding strand, the first oligonucleotide primers would correspond to the coding strand, and the sequences 5′-TAA-3′, 5′-TAG-3′, and 5′-TGA-3′ in the first oligonucleotide primers would ultimately be replicated as stop codons. In some embodiments, therefore, the first oligonucleotide primers, instead of having a complementary sequence to the mutagenic codon, could have any other non-complementary sequence except for 5′-TAA-3′, 5′-TAG-3′, and 5′-TGA-3′.

In some embodiments, one or more of the first oligonucleotide primers in the pool is a mixture of species of oligonucleotide primers having nearly identical sequences. Each species of oligonucleotide primer in such a mixture can have identical sequences except at a position or positions corresponding to a mutagenic site. For example, the mixture can comprise one species of oligonucleotide primer that is fully complementary to a sequence within the target region, and one or more different species of oligonucleotide primers that are complementary to the same sequence within the target region except at a position or positions corresponding to a mutagenic site. In some embodiments, the mixture comprises at least two species of oligonucleotide primers that are complementary to a sequence within the target region except within at least one mutagenic site. In some embodiments, the mixture does not comprise any species of oligonucleotide primer that is fully complementary to a sequence within the target region; in such embodiments, all of the species in the mixture are non-complementary to the mutagenic site at the corresponding position in their sequences.

### Degenerate Primers

In some embodiments, one or more of the first oligonucleotide primers in the pool is a degenerate primer in that it has a degenerate sequence at one or more of the positions corresponding to a mutagenic site. Thus, in embodiments in which the mutagenic site comprises up to three nucleotides, such a degenerate primer is actually a mixture of species of first oligonucleotide primers, wherein the mixture contains more than one possible nucleotide or nucleotide sequence (e.g., dinucleotide, trinucleotide, etc.) at the mutagenic site.

In some embodiments, the degenerate sequence includes all possible nucleotides or nucleotide sequences at a mutagenic site; a primer with such a degenerate sequence may be referred to as a “fully degenerate primer” herein. Generally, “all possible nucleotides” refers to the four standard nucleotides for the appropriate nucleic acid. For deoxyribonucleic acids, the four standard deoxyribonucleotides are deoxyadenylate, deoxyguanylate, deoxythmyidylate, and deoxycytidylate. For ribonucleic acids, the four standard ribonucleotides are adenylate, guanylate, uridylate, and cytidylate. In some embodiments, non-standard nucleotides are used in addition to or instead of standard nucleotides; thus the number of possible nucleotides at a given position may be more than four.

In some embodiments, the degenerate sequence includes a subset of all possible nucleotides or nucleotide sequences at a mutagenic site.

In some embodiments, the degenerate primer contains at least 50% of all of the possible nucleotides or nucleotide sequences at a mutagenic site. In some embodiments, the degenerate primer contains at least 75% of all of the possible nucleotides or nucleotide sequences at a mutagenic site. In some embodiments, the degenerate primer contains at least 80% of all of the possible nucleotides or nucleotide sequences at a mutagenic site. In some embodiments, the degenerate primer contains at least 90% of all of the possible nucleotides or nucleotide sequences at a mutagenic site. In some embodiments, the degenerate primer contains at least 95%, at least 96%, at least 97%, at least 98%, or at least 99% of all of the possible nucleotides or nucleotide sequences at a mutagenic site.

In some embodiments, the degenerate primer includes all of the possible nucleotides or nucleotide sequences at a mutagenic site.

In some embodiments, the degenerate primer includes one species that is fully complementary to a sequence within the target region throughout the entire length of the primer.

In some embodiments, the degenerate primer contains all of the possible nucleotides or nucleotide sequences at a mutagenic site except for the nucleotide or nucleotide sequence that is fully complementary to the corresponding nucleotide or nucleotide sequence in the mutagenic site.

In some embodiments, the degenerate primer has a degenerate sequence at one nucleotide position corresponding to the mutagenic site for the degenerate primer. In some embodiments, the degenerate primer comprises a mixture of four different species of first oligonucleotide primers, wherein the sequences of each species are identical except that they differ by one nucleotide at a position corresponding to the mutagenic site. In some embodiments, the degenerate primer comprises a mixture of three different species of first oligonucleotide primers, wherein the sequences of each species are identical except that they differ by one nucleotide at a position corresponding to the mutagenic site. In some embodiments, the degenerate primer comprises a mixture of at least two different species of first oligonucleotide primers, wherein the sequences of each species are identical except that they differ by one nucleotide at a position corresponding to the mutagenic site.

In some embodiments, the degenerate primer has a degenerate sequence at two nucleotide positions corresponding to the mutagenic site for the degenerate primer. In some embodiments, the degenerate primer comprises a mixture of sixteen different species of first oligonucleotide primers, wherein the sequences of each species are identical except that they differ by at most two nucleotides at positions corresponding to the mutagenic site. In some embodiments, the degenerate primer comprises a mixture of at least eight different species of first oligonucleotide primers, wherein the sequences of each species are identical except that they differ by at most two nucleotides at positions corresponding to the mutagenic site. In some such embodiments, the degenerate primer comprises a mixture of at least nine, at least ten, at least 11, at least 12, at least 13, at least 14, or 15 different species of first oligonucleotide primers, wherein the sequences of each species are identical except that they differ by at most two nucleotides at positions corresponding to the mutagenic site. In some embodiments, the degenerate primer comprises a mixture of 15 different species of first oligonucleotide primers, wherein the sequences of each species are identical except that they differ by at most two nucleotides at positions corresponding to the mutagenic site.

In some embodiments, the degenerate primer has a degenerate sequence at three nucleotide positions corresponding to the mutagenic site for the degenerate primer. In some embodiments, the degenerate primer comprises a mixture of sixty-four different species of first oligonucleotide primers, wherein the sequences of each species are identical except that they differ by at most three nucleotides at positions corresponding to the mutagenic site. In some embodiments, the degenerate primer comprises a mixture of at least thirty-two different species of first oligonucleotide primers, wherein the sequences of each species are identical except that they differ by at most three nucleotides at positions corresponding to the mutagenic site. In some such embodiments, the degenerate primer comprises a mixture of at least 35, at least 40, at least 45, at least 50, at least 55, at least 60, or 63 different species of first oligonucleotide primers, wherein the sequences of each species are identical except that they differ by at most three nucleotides at positions corresponding to the mutagenic site. In some embodiments, the degenerate primer comprises a mixture of 63 different species of first oligonucleotide primers, wherein the sequences of each species are identical except that they differ by at most three nucleotides at positions corresponding to the mutagenic site.

In some embodiments, at least 50% of the first oligonucleotide primers in the pool is a degenerate primer. In some embodiments at least 60%, at least 70%, at least 80, at least 90%, at least 95%, at least 98%, or at least 99% of the first oligonucleotide primers in the pool is a degenerate primer. In some embodiments, all of the first oligonucleotide primers in the pool is a degenerate primer.

In some embodiments, the species of primers within each degenerate primer are present at approximately equimolar concentrations. In some embodiments, the species of primers within each degenerate primer is not present at approximately equimolar concentrations.

### Second Oligonucleotide Primers

In certain embodiments, inventive methods comprise a step of providing a second oligonucleotide primer that is capable of hybridizing to the second strand of the double-stranded template polynucleotide.

In some embodiments, the second oligonucleotide primer is subjected to conditions to allow phosphorylation of oligonucleotide primers. For example, the second oligonucleotide primer can be treated with a kinase. In some embodiments, the kinase is a kinase that phosphorylates DNA substrates at their 5′ ends.

In some embodiments, the second oligonucleotide primer is phosphorylated. In some embodiments, the second oligonucleotide primer is phosphorylated at its 5′ end. In some embodiments, the second oligonucleotide primer is detectably labeled, e.g., at its 5′ end.

In some embodiments, the second oligonucleotide primer is capable of hybridizing to a sequence within the double-stranded template polynucleotide but outside of the target region. In some embodiments, the second oligonucleotide primer is not capable of hybridizing to the target region.

### Third Oligonucleotide Primer

In some embodiments, inventive methods further comprise a step of providing a third oligonucleotide primer, wherein the third oligonucleotide primer is capable of hybridizing to the first strand of the double-stranded template polynucleotide and is 100% complementary to the first strand of the double-stranded template polynucleotide, and wherein step (d) comprises combining the third oligonucleotide primer together with the double-stranded template polynucleotide, pool of first oligonucleotide primers, and second oligonucleotide primer under conditions that allow amplification of the template polynucleotide.

In some embodiments, the third oligonucleotide is 100% complementary to the first strand of the double-stranded template polynucleotide outside of the target region. In some embodiments, the third oligonucleotide primer is capable of hybridizing to a region on the double-stranded template polynucleotide outside of the target region. In some embodiments, the third oligonucleotide primer is not capable of hybridizing to the target region.

In some embodiments in which the template polynucleotide is circular, the third oligonucleotide primer is capable of hybridizing to a region immediately adjacent (e.g., with no intervening nucleotides) to the region to which the second oligonucleotide primer hybridizes. In some embodiments, the second and third oligonucleotide primers, when aligned onto the sequence of the template polynucleotide, are oriented such that, under suitable amplification conditions (e.g., PCR conditions), the third oligonucleotide primer and the second oligonucleotide primer could be used to amplify the entire sequence of the template polynucleotide.

Conditions that Allow Amplification of the Double-Stranded Template Polynucleotide

In certain embodiments, in accordance with certain methods of the invention, the double-stranded template polynucleotide, the pool of first oligonucleotide primers, the second oligonucleotide primer, and optionally the third oligonucleotide primer are combined under conditions that allow amplification of the double-stranded template polynucleotide.

In some embodiments, the first oligonucleotide primers are randomly incorporated during amplification of the template polynucleotide, thereby generating mutated fragments of the template polynucleotide, which may, in some embodiments, recombine into a full length mutated copy of the template polynucleotide (e.g., much in the same way that a linearized destination vector and PCR product prime each other in circular polymerase extension cloning or CPEC (e.g., see Quan and Tiang 2009). In some embodiments, linear DNA could be used as the double-stranded polynucleotide template if homologous ends are designed into the ends of the template such that a circular double-stranded polynucleotide would be created in subsequent cycles.

In certain embodiments, this combining step comprises incubating the double-stranded template polynucleotide, the pool of first oligonucleotide primers, the second oligonucleotide primer, and optionally the third oligonucleotide primer in a reaction mixture.

In some embodiments, the reaction mixture comprises a DNA polymerase. Any of several DNA polymerases may be suitable for use in accordance with inventive methods.

In some embodiments, the DNA polymerase has exonuclease activity. In some embodiments, the DNA polymerase has 3′ to 5′ exonuclease activity.

In some embodiments, the DNA polymerase is not strand-displacing.

In some embodiments, the DNA polymerase has an error rate less than that of Taq polymerase under similar conditions. In some embodiments, the DNA polymerase has an error rate that is at least 10-fold lower, at least 15-fold lower-, at least 20-fold lower, at least 25-fold lower, at least 30-fold lower, at least 35-fold lower, at least 40-fold lower, at least 45-fold lower, or at least 50-fold lower than that of Taq polymerase under similar conditions. In some embodiments, the DNA polymerase has an error rate that is at least 50-fold lower than that of Taq polymerase under similar conditions. In some embodiments, the DNA polymerase has an error rate that is approximately 50-fold lower than that of Taq polymerase under the same conditions.

In some embodiments, the DNA polymerase has an error rate of less than about 3 base pair substitutions per kilobase of DNA. In some embodiments, the DNA polymerase has an error rate of less than about 2 base pair substitutions per kilobase of DNA. In some embodiments, the DNA polymerase has an error rate of less than about 1 base pair substitution per kilobase of DNA. In some embodiments, the DNA polymerase has an error rate of less than about 1 base pair substitution per 2 kilobases of DNA, less than about 1 base pair substitution per 5 kilobases of DNA, less than about 1 base pair substitution per 10 kilobases of DNA, less than about 1 base pair substitution per 50 kilobases of DNA, less than about 1 base pair substitution per 100 kilobases of DNA, less than about 1 base pair substitution per 200 kilobases of DNA, or less than about 1 base pair substitution per 500 kilobases of DNA.

In some embodiments, the DNA polymerase has an error rate of no greater than about 1 base pair per 600 kilobases of DNA, no greater than about 1 base pair per 700 kilobases of DNA, no greater than about 1 base pair per 800 kilobases of DNA, no greater than about 1 base pair per 900 kilobases of DNA, no greater than about 1 base pair per 950 kilobases of DNA, or no greater than about 1 base pair per 1000 kilobases of DNA.

In some embodiments, the DNA polymerase has an error rate of about 1 base pair per 1000 kilobases of DNA.

In some embodiments, the error rate of the DNA polymerase is measured by the polymerase fidelity assay described in Kunkel and Tindall 1988 (hereinafter “Kunkel et al.”), which uses portions of the lacZ gene in M13 bacteriophage to correlate host bacterial colony color changes with errors in DNA synthesis. In some embodiments, the error rate of the DNA polymerase is measured by a modified version of the assay described in Kunkel et al., for example, the assay described in Barnes 1992, which uses PCR to copy the entire lacZ gene and portions of drug resistance genes, with subsequent ligation, cloning, transformation, and counting of blue vs. white colony colors.

In some embodiments, the error rate of the DNA polymerase is measured by an assay that is based on Sanger sequencing of individual cloned PCR products.

Non-limiting examples of suitable DNA polymerases include high fidelity DNA polymerases, such as, e.g., Phusion High Fidelity DNA polymerase (available from Thermo Fisher Scientific), PrimeSTAR GXL DNA Polymerase (available from Clontech), and Q5 High-Fidelity DNA polymerase (available from New England Biolabs).

In some embodiments, the reaction mixture comprises a buffer, e.g., a buffer that is suitable for a DNA polymerase that is being included in the reaction.

In some embodiments, the reaction mixture comprises nucleotides, e.g., deoxyribonucleotides. In some embodiments, the deoxyribonucleotides comprise an equimolar mixture of dATP, dCTP, dGTP, and dTTP.

In some embodiments, the reaction mixture comprises a salt, e.g., magnesium chloride (MgCl₂).

For example, in some embodiments, the reaction mixture comprises a DNA polymerase, deoxyribonucleotides, a buffer and a salt, which, when incubated together with the template polynucleotide, the first oligonucleotide primer, and the second oligonucleotide primer under suitable conditions, will result in amplification of the double-stranded template polynucleotide.

The concentrations of the various components in the reaction mixture can vary depending on the embodiment; and suitable concentrations are often disclosed in known protocols or manufacturer's directions. For example, certain DNA polymerases will perform optimally with certain concentrations of ions such as magnesium ions.

The relative concentrations and absolute amounts of the double-stranded template polynucleotide, pool of first oligonucleotide primers, second oligonucleotide primer, and third oligonucleotide primer (if present) can vary depending on the embodiment.

In some embodiments, at most 500 ng, at most 400 ng, at most 300 ng, at most 200 ng, or at most 100 ng of the double-stranded template polynucleotide is present in the reaction mixture. In some embodiments, at most 200 ng of the double-stranded template polynucleotide is present in the reaction mixture. In some embodiments approximately 200 ng of the double-stranded template polynucleotide is present in the reaction mixture. In some embodiments, at most 100 ng of the double-stranded template polynucleotide is present in the reaction mixture. In some embodiments approximately 100 ng of the double-stranded template polynucleotide is present in the reaction mixture.

In some embodiments, the molar ratio of the pool of first oligonucleotide primers to that of the double-stranded template polynucleotide is approximately 1:1. In some embodiments, the molar ratio of the pool of first oligonucleotide primers to that of the double-stranded template polynucleotide is at least 1:1.

In some embodiments, the pool of first oligonucleotide primers is present in the reaction mixture at a molar excess relative to the concentration of the double-stranded template polynucleotide; i.e., the molar ratio of the pool of first oligonucleotide primers to that of the double-stranded template polynucleotide is greater than 1:1. For example, in some embodiments, the molar ratio of the pool of first oligonucleotide primers to that of the double-stranded template polynucleotide is approximately 1.5:1 or greater. In some embodiments, the molar ratio of the pool of first oligonucleotide primers to that of the double-stranded template polynucleotide is approximately 2:1 or greater, 3:1 or greater, 4:1 or greater, 5:1 or greater, 6:1 or greater, 7:1 or greater, 8:1 or greater, 9:1 or greater, or 10:1 or greater. In some embodiments, the molar ratio of the pool of first oligonucleotide primers to that of the double-stranded template polynucleotide is approximately 15:1 or greater, 20:1 or greater, 25:1 or greater, 30:1 or greater, 35:1 or greater, 40:1 or greater, 45:1 or greater, 50:1 or greater, 55:1 or greater, 60:1 or greater, 65:1 or greater, 70:1 or greater, 75:1 or greater, 80:1 or greater, 85:1 or greater 90:1 or greater, or 95:1 or greater, or 100:1 or greater. In some embodiments, the molar ratio of the pool of first oligonucleotide primers to that of the double-stranded template polynucleotide is approximately 10:1 or greater. In some embodiments, the molar ratio of the pool of first oligonucleotide primers to that of the double-stranded template polynucleotide is approximately 50:1 or greater. In some embodiments, the molar ratio of the pool of first oligonucleotide primers to that of the double-stranded template polynucleotide is approximately 100:1 or greater.

In some embodiments, the molar ratio of the second oligonucleotide primer to that of the double-stranded template polynucleotide is approximately 1:1. In some embodiments, the molar ratio of the second oligonucleotide primer to that of the double-stranded template polynucleotide is at least 1:1.

In some embodiments, the second oligonucleotide primer is present in the reaction mixture at a molar excess relative to the concentration of the double-stranded template polynucleotide; i.e., the molar ratio of the second oligonucleotide primer to that of the double-stranded template polynucleotide is greater than 1:1. For example, in some embodiments, the molar ratio of the second oligonucleotide primer to that of the double-stranded template polynucleotide is approximately 1.5:1 or greater. In some embodiments, the molar ratio of the second oligonucleotide primer to that of the double-stranded template polynucleotide is approximately 2:1 or greater, 3:1 or greater, 4:1 or greater, 5:1 or greater, 6:1 or greater, 7:1 or greater, 8:1 or greater, 9:1 or greater, or 10:1 or greater. In some embodiments, the molar ratio of the second oligonucleotide primer to that of the double-stranded template polynucleotide is approximately 15:1 or greater, 20:1 or greater, 25:1 or greater, 30:1 or greater, 35:1 or greater, 40:1 or greater, 45:1 or greater, 50:1 or greater, 55:1 or greater, 60:1 or greater, 65:1 or greater, 70:1 or greater, 75:1 or greater, 80:1 or greater, 85:1 or greater 90:1 or greater, or 95:1 or greater, or 100:1 or greater. In some embodiments, the molar ratio of the second oligonucleotide primer to that of the double-stranded template polynucleotide is approximately 10:1 or greater. In some embodiments, the molar ratio of the second oligonucleotide primer to that of the double-stranded template polynucleotide is approximately 50:1 or greater. In some embodiments, the molar ratio of the second oligonucleotide primer to that of the double-stranded template polynucleotide is approximately 100:1 or greater.

In some embodiments, the third oligonucleotide primer is present in the reaction mixture at a concentration that is approximately equimolar to that of the double-stranded template polynucleotide, i.e., in a 1:1 ratio.

In some embodiments, the third oligonucleotide primer is present in the reaction mixture at a molar excess relative to the concentration of the double-stranded template polynucleotide; i.e., the molar ratio of the third oligonucleotide primer to that of the double-stranded template polynucleotide is greater than 1:1. For example, in some embodiments, the molar ratio of the third oligonucleotide primer to that of the double-stranded template polynucleotide is approximately 1.5:1 or greater. In some embodiments, the molar ratio of the third oligonucleotide primer to that of the double-stranded template polynucleotide is approximately 2:1 or greater, 3:1 or greater, 4:1 or greater, 5:1 or greater, 6:1 or greater, 7:1 or greater, 8:1 or greater, 9:1 or greater, or 10:1 or greater. In some embodiments, the molar ratio of the third oligonucleotide primer to that of the double-stranded template polynucleotide is approximately 15:1 or greater, 20:1 or greater, 25:1 or greater, 30:1 or greater, 35:1 or greater, 40:1 or greater, 45:1 or greater, 50:1 or greater, 55:1 or greater, 60:1 or greater, 65:1 or greater, 70:1 or greater, 75:1 or greater, 80:1 or greater, 85:1 or greater 90:1 or greater, or 95:1 or greater, or 100:1 or greater. In some embodiments, the molar ratio of the third oligonucleotide primer to that of the double-stranded template polynucleotide is approximately 10:1 or greater. In some embodiments, the molar ratio of the third oligonucleotide primer to that of the double-stranded template polynucleotide is approximately 50:1 or greater. In some embodiments, the molar ratio of the third oligonucleotide primer to that of the double-stranded template polynucleotide is approximately 100:1 or greater.

In some embodiments, the double-stranded template polynucleotide is present in the reaction mixture at a molar excess relative to the concentration of the third oligonucleotide primer; i.e., the molar ratio of the double-stranded template polynucleotide to that of the third oligonucleotide primer is greater than 1:1. For example, in some embodiments, the molar ratio of the double-stranded template polynucleotide to that of the third oligonucleotide primer is approximately 1.5:1 or greater. In some embodiments, the molar ratio of the double-stranded template polynucleotide to that of the third oligonucleotide primer is approximately 2:1 or greater, 3:1 or greater, 4:1 or greater, 5:1 or greater, 6:1 or greater, 7:1 or greater, 8:1 or greater, 9:1 or greater, or 10:1 or greater. In some embodiments, the molar ratio of the double-stranded template polynucleotide to that of the third oligonucleotide primer is approximately 15:1 or greater, 20:1 or greater, 25:1 or greater, 30:1 or greater, 35:1 or greater, 40:1 or greater, 45:1 or greater, 50:1 or greater, 55:1 or greater, 60:1 or greater, 65:1 or greater, 70:1 or greater, 75:1 or greater, 80:1 or greater, 85:1 or greater 90:1 or greater, or 95:1 or greater, or 100:1 or greater. In some embodiments, the molar ratio of the double-stranded template polynucleotide to that of the third oligonucleotide primer is approximately 10:1 or greater. In some embodiments, the molar ratio of the double-stranded template polynucleotide to that of the third oligonucleotide primer is approximately 50:1 or greater. In some embodiments, the molar ratio of the double-stranded template polynucleotide to that of the third oligonucleotide primer is approximately 100:1 or greater.

In some embodiments, the pool of first oligonucleotide primers is present in the reaction mixture at a concentration that is approximately equimolar to that of the second oligonucleotide primer.

### Amplification Conditions

In accordance with certain methods of the invention, the template polynucleotide is amplified such that one or more mutant copies of the template polynucleotide is generated.

In certain embodiments, conditions that allow amplification of the template polynucleotide comprise incubating a reaction mixture through one or more cycles of different temperatures (“thermocycling”). In some embodiments, such conditions are similar to or the same as conditions suitable for polymerase chain reactions. For example, a reaction mixture can undergo one or more thermocycles, each thermocycle comprising incubating the reaction mixture at 1) a temperature suitable for denaturation of double-stranded polynucleotide complexes for a period of time (the “denaturation phase”), 2) then at a temperature suitable for annealing of two strands of polynucleotides and/or oligonucleotides (e.g., annealing of oligonucleotides to a polynucleotide) for a period of time (the “annealing phase”), and 3) then at a temperature suitable for extension of an oligonucleotide primer by one or more nucleotides for a period of time (the “extension phase”), wherein the periods of time during the denaturation phase, the annealing phase, and the extension phase may be different or the same.

In some embodiments, the reaction mixture is incubated for an initial period of time at the temperature suitable for denaturation before the set of thermocycles.

In some embodiments, the reaction mixture is incubated for a final period of time at the temperature suitable for extension after the set of thermocycles.

The temperatures, periods of time for each temperature, and total of number of thermocycles may vary depending on the embodiment and/or may be influenced by factors such as the length of the template polynucleotide, complexity of the template polynucleotide sequence, type of DNA polymerase used, etc. For example, a longer extension phase (e.g., longer than the denaturation phase and/or the annealing phase) might be used when trying to amplify a larger double-stranded template polynucleotide.

In general, one of ordinary skill in the art would be able to adjust the conditions for amplification accordingly.

In some embodiments, the temperature used during the denaturation phase (the “denaturation temperature”) is within five degrees Celsius of 98° C., within four degrees Celsius of 98° C., within three degrees Celsius of 98° C., within two degrees Celsius of 98° C., or within one degree Celsius of 98° C. In some embodiments, the denaturation temperature is approximately 98° C.

In some embodiments, the temperature used during the annealing phase (the “annealing temperature”) is within five degrees Celsius of 55° C., within four degrees Celsius of 55° C., within three degrees Celsius of 55° C., within two degrees Celsius of 55° C., or within one degree Celsius of 55° C. In some embodiments, the annealing temperature is approximately 55° C.

In some embodiments, the temperature used during the extension phase (the “extension temperature”) is within five degrees Celsius of 72° C., within four degrees Celsius of 72° C., within three degrees Celsius of 72° C., within two degrees Celsius of 72° C., or within one degree Celsius of 72° C. In some embodiments, the extension temperature is approximately 72° C.

In some embodiments, the length of the denaturation phase (the “denaturation period”) is greater than about 15 seconds, greater than about 20 seconds, or greater than about 25 seconds. In some embodiments, the denaturation period is less than about 50 seconds, less than about 45 seconds, less than about 40 seconds, or less than about 30 seconds. In some embodiments, the denaturation period is about 30 seconds.

In some embodiments, the length of the annealing phase (the “annealing period”) is greater than about 15 seconds, greater than about 20 seconds, or greater than about 25 seconds. In some embodiments, the annealing period is less than about 50 seconds, less than about 45 seconds, less than about 40 seconds, or less than about 30 seconds. In some embodiments, the annealing period is about 30 seconds.

The length of the extension phase (the “extension period”) can be varied, e.g., depending on the length of the double-stranded template polynucleotide. Generally, a longer extension period may be suitable for longer double-stranded template polynucleotides.

In some embodiments, the extension period is greater than about 60 seconds, greater than about 90 seconds, greater than about 2 minutes, greater than about 2.5 minutes, greater than about 3 minutes, greater than about 3.5 minutes, greater than about 4 minutes, greater than about 4.5 minutes, greater than about 5 minutes, greater than about 5.5 minutes, greater than about 6 minutes, greater than about 6.5 minutes, greater than about 7 minutes, greater than about 7.5 minutes, greater than about 8 minutes, greater than about 8.5 minutes, or greater than about 9 minutes.

In some embodiments, the extension period is less than about 20 minutes, less than about 19 minutes, less than about 18 minutes, less than about 17 minutes, less than about 16 minutes, less than about 15 minutes, less than about 14 minutes, less than about 13 minutes, less than about 12 minutes, or less than about 11 minutes. In some embodiments, the extension period is about 10 minutes.

### Nuclease Incubation

In some embodiments involving a reaction mixture, inventive methods further comprise a step of incubating the reaction mixture with one or more nucleases under conditions to allow activity of the one or more nucleases. Such a step may be useful, for example, in digesting (and thereby facilitating removal of and/or reduction in the amount of) undesired products, such as, for example, template polynucleotides, linear polynucleotides (e.g., in embodiments in which the desired products are circular), and/or single-stranded polynucleotides (e.g., in embodiments in which the desired products are double-stranded).

In some embodiments, the one or more nucleases comprises a methylation-specific nuclease, an exonuclease, a nuclease specific for single-stranded DNA, or a combination thereof.

A “methylation-specific” nuclease preferentially cleaves hemimethylated and/or fully methylated nucleic acid substrates over unmethylated nucleic acid substrates. The methylation-specific nuclease can be useful, for example, in digesting template polynucleotides that have been produced in organisms that methylate DNA. For example, plasmid DNA produced in Escherichia coli is typically methylated; thus, if such plasmid DNA is used as a template polynucleotide, the template polynucleotide can be removed using a nuclease that is specific for methylated DNA. Non-limiting examples of methylation-specific nucleases include DpnI, FspEI, LpnPI, McrBC, and MspJI. In some embodiments, the methylation-specific nuclease is DpnI. In some embodiments, a mixture of more than one methylation-specific nuclease is used.

Exonucleases cleave phosphodiester bonds at the ends of polynucleotides, e.g., at the 5′ and/or 3′ end. Exonucleases therefore may be useful in digesting linear polynucleotides, for example, in embodiments where the desired products are non-linear, e.g., circular.

In some embodiments, the exonuclease is processive, i.e., it is able to cleave phosphodiester bonds consecutively without releasing its substrate.

In some embodiments, the exonuclease cannot cleave nicked circular double-stranded DNA.

In some embodiments, the exonuclease preferentially cleaves double-stranded substrates over single-stranded substrates.

In some embodiments, the exonuclease preferentially cleaves substrates that are phosphorylated at the 5′ end. In some such embodiments, the first oligonucleotide primers and/or second oligonucleotide primer is/are phosphorylated at the 5′ end, thus “tagging” any linear polynucleotides for cleavage by the exonuclease.

Any of a variety of exonucleases may be suitable for use in accordance with methods of the invention. In some embodiments, the exonuclease is lambda exonuclease. In some embodiments, the exonuclease is Exo III, Exo V or Exo VIII. In some embodiments, the exonuclease is Exo V. In some embodiments, the exonuclease is Exo VIII.

In some embodiments, the one or more nucleases with which the reaction mixture is incubated includes a nuclease specific for single-stranded DNA. In some embodiments, the nuclease specific for single-stranded DNA removes nucleotides from single-stranded DNA in the 3′ to 5′ direction. In some embodiments, the nuclease specific for single-stranded DNA is Exo I. In some embodiments, the nuclease specific for single-stranded DNA is mung bean nuclease. In some embodiments, the nuclease specific for single-stranded DNA is RecJf. In some embodiments, the nuclease specific for single-stranded DNA is Exo T.

In some embodiments in which the reaction mixture is incubated with more than one nuclease, one incubation step is carried out with all the nucleases together.

In some embodiments in which the reaction mixture is incubated with more than one nuclease, more than one incubation step is carried out, e.g., sequentially. For example, each incubation step can involve a subset of the more than one nuclease, with each incubation step being carried under the conditions that are most suitable for nucleases that are involved at that step. In some embodiments, an inactivation step is carried out in between at least some of the incubation steps, e.g., to inactivate the nuclease(s) used in the previous step. In some embodiments, a cleanup step is carried out in between at least some of the incubation steps, e.g., to remove some of the cleavage products generated and/or nuclease(s) used in the previous step.

### Tuning the Mutagenesis Rate

In accordance with methods disclosed herein, the rate of mutagenesis can be tuned by varying certain parameters. The rate of mutagenesis can be measured in any of a variety of ways, a non-limiting example of which is the average number of codon mutations in each mutated polynucleotide.

For example, including a third oligonucleotide primer as described herein during the amplification of the double-stranded template polynucleotide can lower the mutation rate. Further tuning can be achieved by, for example, altering the molar ratio of the third oligonucleotide primer relative to that of other components present during amplification.

In some embodiments, the third oligonucleotide primer is present in the reaction mixture at a concentration equimolar to that of the double-stranded template polynucleotide.

In some embodiments, the third oligonucleotide primer is present in the reaction mixture at a concentration equimolar to that of the pool of first oligonucleotide primers. In some embodiments, the third oligonucleotide primer is present in the reaction mixture at a lower concentration that that of the pool of first oligonucleotide primers. For example, in some embodiments, the molar ratio of the third oligonucleotide primer to that of the pool of first oligonucleotide primers is approximately 1:10 or less, approximately 1:30 or less, approximately 1:40 or less, approximately 1:50 or less, approximately 1:60 or less, approximately 1:70 or less, approximately 1:80 or less, approximately 1:90 or less, approximately 1:100 or less, approximately 1:120 or less, approximately 1:140 or less, approximately 1:160 or less, approximately 1:180 or less, or approximately 1:200 or less. In some embodiments, the molar ratio of the third oligonucleotide primer to that of the pool of first oligonucleotide primers is approximately 1:100 or less. In some embodiments, the molar ratio of the third oligonucleotide primer to that of the pool of first oligonucleotide primers is approximately 1:100.

In some embodiments, the third oligonucleotide primer is present in the reaction mixture at a concentration equimolar to that of the second oligonucleotide primers. In some embodiments, the third oligonucleotide primer is present in the reaction mixture at a lower concentration that that of the second oligonucleotide primer. For example, in some embodiments, the molar ratio of the third oligonucleotide primer to that of the second oligonucleotide primer is approximately 1:10 or less, approximately 1:30 or less, approximately 1:40 or less, approximately 1:50 or less, approximately 1:60 or less, approximately 1:70 or less, approximately 1:80 or less, approximately 1:90 or less, approximately 1:100 or less, approximately 1:120 or less, approximately 1:140 or less, approximately 1:160 or less, approximately 1:180 or less, or approximately 1:200 or less. In some embodiments, the molar ratio of the third oligonucleotide primer to that of the second oligonucleotide primer is approximately 1:100 or less. In some embodiments, the molar ratio of the third oligonucleotide primer to that of the second oligonucleotide primers is approximately 1:100.

### Libraries

In certain aspects, provided are libraries of mutant copies of a template polynucleotide, wherein each mutant copy includes a mutated version of at least a portion of the target region (i.e., a region targeted for mutagenesis). In some embodiments, provided libraries are generated by methods which start from a double-stranded template polynucleotide as disclosed herein. A mutant copy of a double-stranded template polynucleotide can include a copy of all or just a portion of one strand of the template polynucleotide. A mutant copy of a double-stranded template polynucleotide can also include a copy of all or just a portion of both strands of the template polynucleotide.

It should be understood that although provided libraries contain mutant copies of a template polynucleotide, and may well largely comprise mutant copies, it may also contain copies that are not mutated, e.g., having identical sequence (“wildtype”) as that of the template polynucleotide. In some embodiments, the wildtype percentage in the library is less than 25%, for example, less than 20%, less than 15%, less than 10%, less than 9%, less than 8%, less than 7%, less than 6%, less than 5%, less than 4%, less than 3%, less than 2%, or less than 1%. It is to be understood that the term “wildtype” is used herein to refer to the sequence of the template polynucleotide that is used as the starting point for mutagenesis and that it need not be a naturally occurring sequence.

Typically, many mutants are generated by inventive methods of mutagenesis. Typically, the larger the target region, the larger the size of the library and the greater the number of mutant copies contained in the library.

In some embodiments, the library contains at least 10, at least 20, at least 30, at least 40, at least 50, at least 60, at least 70, at least 80, or at least 90 different mutant copies. In some embodiments, the library contains at least 100, at least 200, at least 300, at least 400, at least 500, at least 600, at least 700, at least 800, or at least 900 different mutant copies. In some embodiments, the library contains at least 1,000, at least 2,000, at least 3,000, at least 4,000, at least 5,000, at least 6,000, at least 7,000, at least 8,000, or at least 9,000 different mutant copies. In some embodiments, the library contains at least 10,000, at least 20,000, at least 30,000, at least 40,000, at least 50,000, at least 60,000, at least 70,000, at least 80,000, or at least 90,000 different mutant copies. In some embodiments, the library contains at least 100,000 different mutant copies.

In some embodiments, the library includes a plurality of mutant copies that include a copy of all of one or both strands of the template polynucleotide where the sequences of the mutant copies are identical in length as the template polynucleotide and only differ from the sequence of the template polynucleotide at the mutation or mutations that were introduced as a result of the methods.

In some embodiments, the library is a plasmid library.

When the template polynucleotide encodes a polypeptide, the number of amino acids represented at a given codon of the polypeptide can be used as a measure of the extent of the library's coverage of the mutant space, or the library's diversity. Similarly, the number or percentage of codons for which there is at least one mutant (relative to the template polynucleotide) in the library can be used as a measure of the extent of the library's coverage of the mutant space, or the library's diversity.

In some embodiments, the library comprises at least one mutant for at least 50%, at least 60%, at least 65%, at least 70%, at least 75%, at least 80%, at least 85%, at least 90%, at least 95%, at least 97%, at least 98%, at least 99%, or all of the codons in the target region. In some embodiments, the library comprises at least one mutant for at least 75% of the codons in the target region. In some embodiments, the library comprises at least one mutant for at least 90% of the codons in the target region. In some embodiments, the library comprises at least one mutant for at least 95% of the codons in the target region. In some embodiments, the library comprises at least one mutant for at least 97% of the codons in the target region. In some embodiments, the library comprises at least one mutant for at least 98% of the codons in the target region. In some embodiments, the library comprises at least one mutant for at least 99% of the codons in the target region. In some embodiments, the library comprises at least one mutant for all of the codons in the target region.

In some embodiments, the average mutation rate across the library is at least 1 codon mutation per mutated polynucleotide, for example, at least 2 codon mutations per mutated polynucleotide, at least 3 codon mutations per mutated polynucleotide, at least 4 codon mutations per mutated polynucleotide, at least 5 codon mutations per mutated polynucleotide, at least 6 codon mutations per mutated polynucleotide, at least 7 codon mutations per mutated polynucleotide, at least 8 codon mutations per mutated polynucleotide, at least 9 codon mutations per mutated polynucleotide, or at least 10 codon mutations per mutated polynucleotide.

In some embodiments, the median mutation rate across the library is at least 1 codon mutation per mutated polynucleotide, for example, at least 2 codon mutations per mutated polynucleotide, at least 3 codon mutations per mutated polynucleotide, at least 4 codon mutations per mutated polynucleotide, at least 5 codon mutations per mutated polynucleotide, at least 6 codon mutations per mutated polynucleotide, at least 7 codon mutations per mutated polynucleotide, at least 8 codon mutations per mutated polynucleotide, at least 9 codon mutations per mutated polynucleotide, or at least 10 codon mutations per mutated polynucleotide.

In some embodiments, at least 5, 10, 15, 16, 17, 18, or 19 different amino acids are represented at a given codon for at least 50% of the codons in the target region. In some embodiments, 20 different amino acids are represented at a given codon for at least 50% of the codons in the target region.

In some embodiments, at least 5, 10, 15, 16, 17, 18, or 19 different amino acids are represented at a given codon for at least 60% of the codons in the target region. In some embodiments, 20 different amino acids are represented at a given codon for at least 60% of the codons in the target region.

In some embodiments, at least 5, 10, 15, 16, 17, 18, or 19 different amino acids are represented at a given codon for at least 65% of the codons in the target region. In some embodiments, 20 different amino acids are represented at a given codon for at least 65% of the codons in the target region.

In some embodiments, at least 5, 10, 15, 16, 17, 18, or 19 different amino acids are represented at a given codon for at least 70% of the codons in the target region. In some embodiments, 20 different amino acids are represented at a given codon for at least 70% of the codons in the target region.

In some embodiments, at least 5, 10, 15, 16, 17, 18, or 19 different amino acids are represented at a given codon for at least 75% of the codons in the target region. In some embodiments, 20 different amino acids are represented at a given codon for at least 75% of the codons in the target region.

In some embodiments, at least 5, 10, 15, 16, 17, 18, or 19 different amino acids are represented at a given codon for at least 80% of the codons in the target region. In some embodiments, 20 different amino acids are represented at a given codon for at least 80% of the codons in the target region.

In some embodiments, at least 5, 10, 15, 16, 17, 18, or 19 different amino acids are represented at a given codon for at least 85% of the codons in the target region. In some embodiments, 20 different amino acids are represented at a given codon for at least 85% of the codons in the target region.

In some embodiments, at least 5, 10, 15, 16, 17, 18, or 19 different amino acids are represented at a given codon for at least 90% of the codons in the target region. In some embodiments, 20 different amino acids are represented at a given codon for at least 90% of the codons in the target region.

In some embodiments, at least 5, 10, 15, 16, 17, 18, or 19 different amino acids are represented at a given codon for at least 95% of the codons in the target region. In some embodiments, 20 different amino acids are represented at a given codon for at least 95% of the codons in the target region.

In some embodiments, at least 5, 10, 15, 16, 17, 18, or 19 different amino acids are represented at a given codon for at least 96% of the codons in the target region. In some embodiments, 20 different amino acids are represented at a given codon for at least 96% of the codons in the target region.

In some embodiments, at least 5, 10, 15, 16, 17, 18, or 19 different amino acids are represented at a given codon for at least 97% of the codons in the target region. In some embodiments, 20 different amino acids are represented at a given codon for at least 97% of the codons in the target region.

In some embodiments, at least 5, 10, 15, 16, 17, 18, or 19 different amino acids are represented at a given codon for at least 98% of the codons in the target region. In some embodiments, 20 different amino acids are represented at a given codon for at least 98% of the codons in the target region.

In some embodiments, at least 5, 10, 15, 16, 17, 18, or 19 different amino acids are represented at a given codon for at least 99% of the codons in the target region. In some embodiments, 20 different amino acids are represented at a given codon for at least 99% of the codons in the target region.

In some embodiments, at least 5, 10, 15, 16, 17, 18, or 19 different amino acids are represented at a given codon for all of the codons in the target region. In some embodiments, 20 different amino acids are represented at a given codon for all of the codons in the target region.

In some embodiments, very little or no bias for or against particular codons is observed across all the mutants in the library. The number, fraction, or percentage of mutant copies having a mutation in each codon can give a measure of the bias the library has for or against particular codons. For example, when using a pool of first oligonucleotide primers that are fully degenerate (i.e., encompass A, G, T and C at each position within the target region), the level of bias can be assessed by calculating an average frequency of each different base observed at each position within the target region (where an average of 0.25 would equate to no bias). In some such embodiments, the average frequency of different bases observed at each position within the target region is between 0.25 and 0.35, for example, between 0.25 and 0.5, between 0.25 and 0.75, or between 0.25 and 0.9. In some such embodiments, the average frequency of different bases observed at each position within the target region is greater than 0.5, for example, greater than 0.75, or greater than 0.9.

In some embodiments, for every codon in the target region, the library comprises no more than x mutant copies having a mutation in that codon, wherein

$$x = j \times \frac{p}{n} , \qquad ( Equation 1 )$$

wherein n denotes the number of codons in the target region, p denotes the total number of mutant copies in the library, and j is selected from 2, 1.9, 1.8. 1.6, 1.5, 1.4, 1.3, 1.2, and 1.1. In some embodiments, j is 2. In some embodiments, j is 1.5. In some embodiments, j is 1.2. In some embodiments, j is 1.1.

In some embodiments, for every codon in the target region, the library comprises no fewer than x mutant copies having a mutation in that codon, wherein

$$x = \frac{p}{kn} , \qquad ( Equation 2 )$$

wherein n denotes the number of codons in the target region, p denotes the total number of mutant copies in the library, and k is selected from 2, 1.9, 1.8. 1.6, 1.5, 1.4, 1.3, 1.2, and 1.1. In some embodiments, k is 2. In some embodiments, k is 1.5. In some embodiments, k is 1.2. In some embodiments, k is 1.1.

In certain embodiments, the library of mutant copies is transformed into cells. For example, in embodiments in which the mutant copies are plasmids, the plasmids are transformed into cells that can replicate such plasmids, e.g., bacterial cells.

### Uses

In some embodiments, libraries generated by methods of the present invention are used in selections to select for mutated copies having, or encoding mutated polypeptides having, certain desirable properties (“positive selection”) and/or against mutated copies having, or encoding mutated polypeptides having, certain undesirable properties (“negative selection”).

In some embodiments, mutant copies that been selected after undergoing one or more selection protocols are further mutated using methods of the present invention. This cycle (mutagenesis to generate a library of mutant copies followed by one or more selection rounds) can be repeated as many times as desired (e.g., 2, 3, 4, 5, 10, 20 or more times), for example, until the mutant copies obtained meet certain criteria and/or a desired number of mutant copies meeting certain criteria are obtained.

The conditions for mutagenesis during each round of mutagenesis need not be the same, nor do the conditions for the selection need to be the same. For example, in one cycle, the target region within the double-stranded template polynucleotide may span, e.g., an entire coding region, while in another cycle, the target region within the template polynucleotide may span, e.g., only a subset of the codons of a coding region such as the codons that code for a particular active site of the polypeptide.

Although the inventive methods allow methods of preparing libraries of mutant copies with little or no bias for or against particular sub-regions within the target region (e.g., little or no bias for or against particular codons within a target region), it is possible that after one or more rounds of selection, the collection of selected mutant copies may exhibit a bias for particular sub-regions and/or codons.

Exemplary Target Regions that Encode all or a Portion of a Cas Molecule

As discussed above, the methods of the present invention are generally applicable to any polynucleotide sequence that one desires to mutagenize and/or for which one desires to generate a library of mutants. In this section we discuss target regions that encode all or a portion of a Cas molecule (specifically a Cas9 molecule) to illustrate some exemplary target regions that can be mutated in accordance with the methods of the present invention. It will be appreciated that these embodiments are provided solely for illustrative purposes and that the methods can readily be applied to target regions from other types of Cas molecules (e.g., Cpf1 molecules and C2c1 or C2c3 molecules) but also to target regions from other types of nucleases (e.g., meganucleases, TALENs, zinc finger nucleases, etc.) and to target regions from other types of polypeptides (e.g., antibodies, etc.) or target regions from other molecules that are encoded by a double-stranded template polynucleotide of the present invention (e.g., RNA aptamers, gRNAs, etc.).

### Cas Molecules

CRISPR-associated (Cas) molecules are guided to specific sites by “guide RNAs” or gRNAs. In some embodiments, the target region encodes all or a portion of a CRISPR-associated (Cas) molecule, a fragment thereof, or a variant thereof (e.g., a catalytically inactive variant or a “nickase” variant which has been mutated in such a way that it only cleaves a single strand of double-stranded DNA). In some embodiments, the target region encodes all or a portion of a Cas molecule, and a nucleotide sequence that serves as a template for a gRNA suitable for guiding the Cas molecule is also included in the double-stranded template polynucleotide. In some such embodiments, all or a portion of the nucleotide sequence that serves as a template for the gRNA is also part of the target region (i.e., leading to the production of a library that can be used to simultaneously produce mutated Cas molecules and mutated gRNA molecules). In some such embodiments, the nucleotide sequence that serves as a template for the gRNA is not part of the target region.

Various types of Cas molecules or Cas polypeptides can be used to practice the inventions disclosed herein. In some embodiments, Cas molecules of Type II Cas systems are used (e.g., Cas9 molecules). In other embodiments, Cas molecules of other Cas systems are used. Exemplary Cas molecules (and Cas systems) have been described previously (see, e.g., Haft 2005, Makarova 2011 and Makarova 2015). For example, in some embodiments Cas molecules from Class 1 Cas systems are used including Cas molecules from the Type I, Type III and Type IV Cas systems (see, e.g., Makarova 2015). In some embodiments Cas molecules from Class 2 Cas systems may be used including Cas molecules from the Type II or Type V Cas systems (e.g., Cas9 molecules but also Cpf1 molecules and C2c1 or C2c3 molecules) (see, e.g., Zetsche 2015 and Shmakov 2015).

### Cas9 Molecules

In some embodiments, the target region encodes all or a portion of a Cas9 molecule. Target regions which encode all of a portion of a Cas9 molecule from a variety of species can be used in the methods and compositions described herein. While S. pyogenes and S. aureus Cas9 molecules are the subject of much of the disclosure herein, Cas9 molecules of, derived from, or based on the Cas9 proteins of other species can be used as well.

Exemplary naturally occurring Cas9 molecules have been described previously (see, e.g., Chylinski 2013). Exemplary naturally occurring Cas9 molecules include a Cas9 molecule of a cluster 1 bacterial family. Examples include a Cas9 molecule of: S. aureus, S. pyogenes (e.g., strain SF370, MGAS10270, MGAS10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA159, NN2025), S. macacae (e.g., strain NCTC11558), S. gallolyticus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. anginosus (e.g., strain F0211), S. agalactiae (e.g., strain NEM316, A909), Listeria monocytogenes (e.g., strain F6854), Listeria innocua (L. innocua, e.g., strain Clip11262), Enterococcus italicus (e.g., strain DSM 15952), or Enterococcus faecium (e.g., strain 1,231,408).

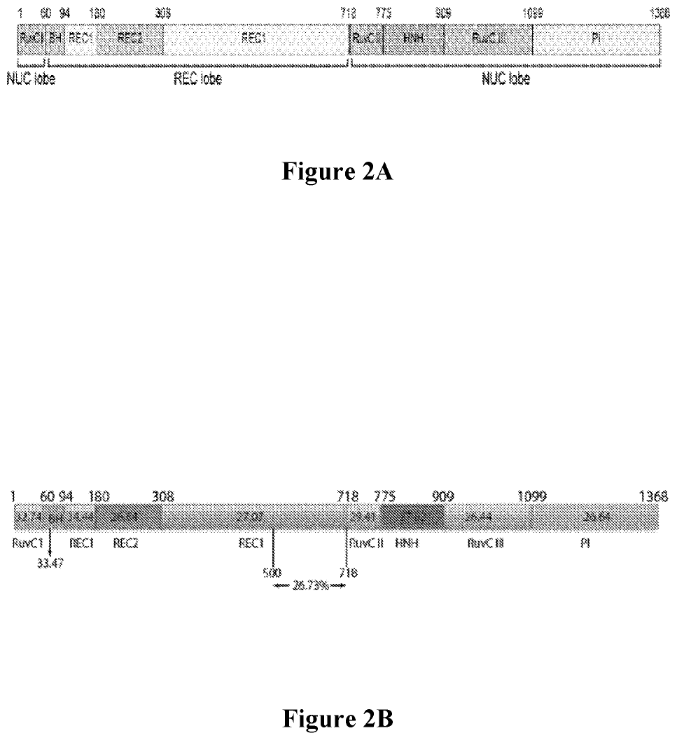

Crystal structures have been determined for two different naturally occurring bacterial Cas9 molecules (Jinek 2014) and for S. pyogenes Cas9 with a guide RNA (e.g., a synthetic fusion of crRNA and tracrRNA) (Nishimasu 2014; Anders 2014). A naturally occurring Cas9 molecule comprises two lobes: a recognition (REC) lobe and a nuclease (NUC) lobe; each of which further comprise domains described herein. FIGS. 2A-2B provide a schematic of the organization of important Cas9 domains in the primary structure. The domain nomenclature and the numbering of the amino acid residues encompassed by each domain used throughout this disclosure is as described previously (Nishimasu 2014). The numbering of the amino acid residues is with reference to Cas9 from S. pyogenes.

The REC lobe comprises the arginine-rich bridge helix (BH), the REC1 domain, and the REC2 domain. The REC lobe does not share structural similarity with other known proteins, indicating that it is a Cas9-specific functional domain. The BH domain is a long a helix and arginine rich region and comprises amino acids 60-93 of the sequence of S. pyogenes Cas9. The REC1 domain is important for recognition of the repeat:anti-repeat duplex, e.g., of a gRNA or a tracrRNA, and is therefore critical for Cas9 activity by recognizing the target sequence. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat:anti-repeat duplex. The REC2 domain comprises amino acids 180-307 of the sequence of S. pyogenes Cas9.

The NUC lobe comprises the RuvC domain, the HNH domain, and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule, and comprises amino acids 1099-1368 of the sequence of S. pyogenes Cas9.

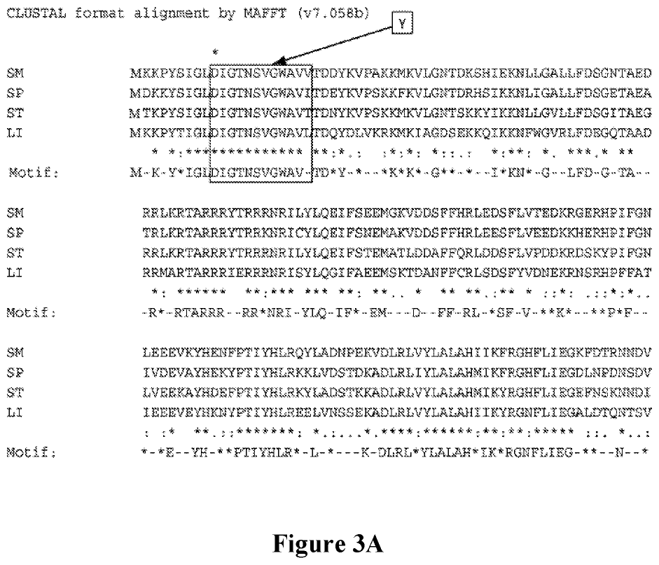

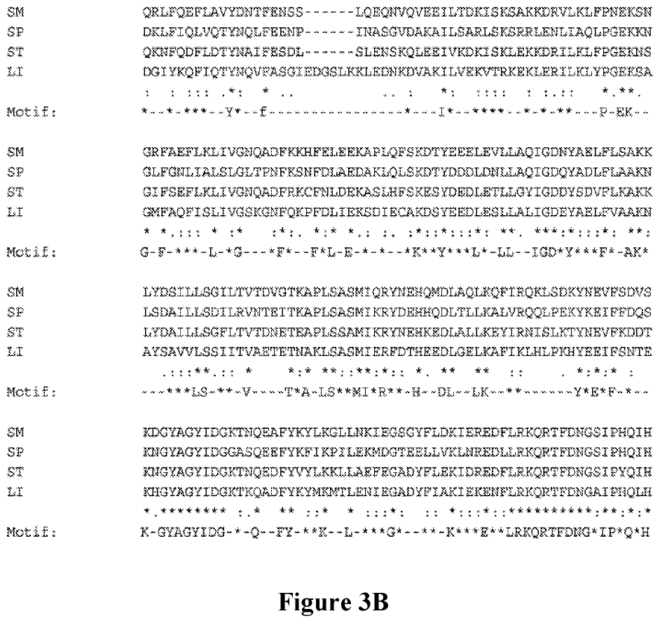

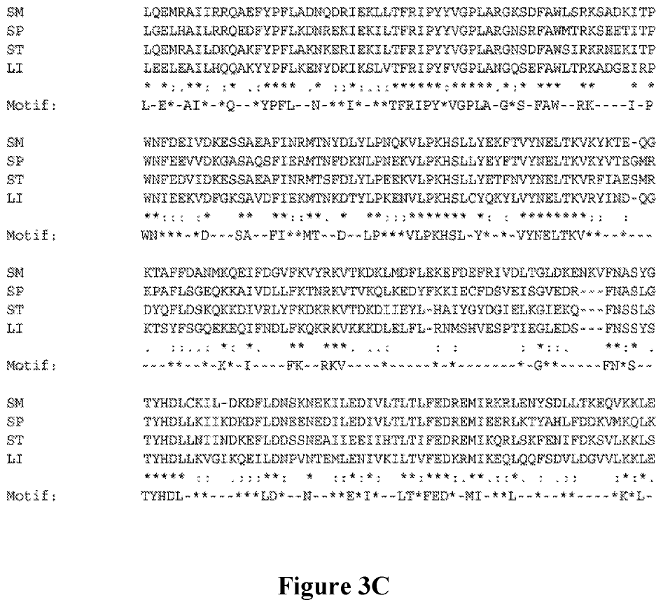

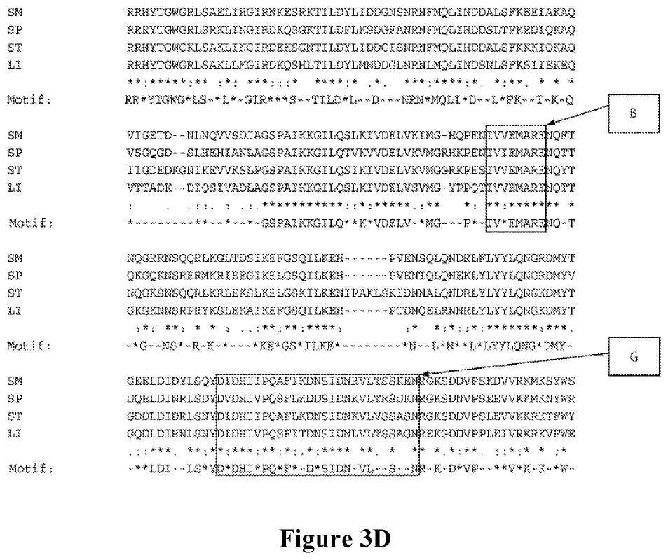

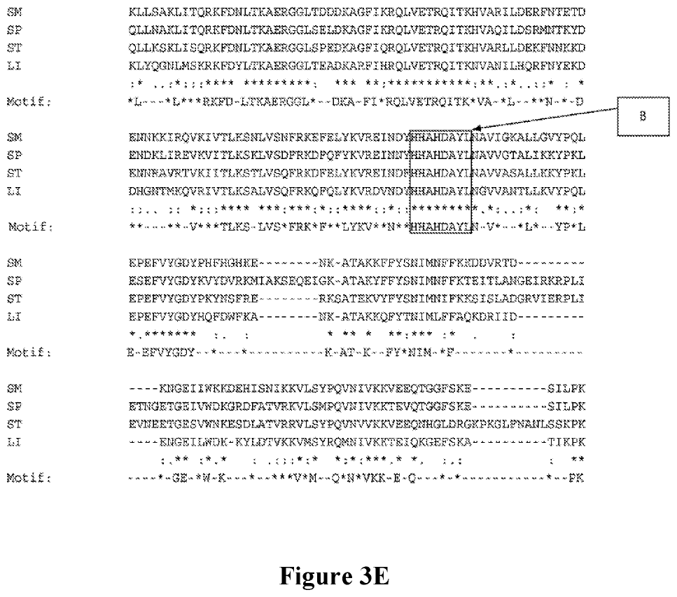

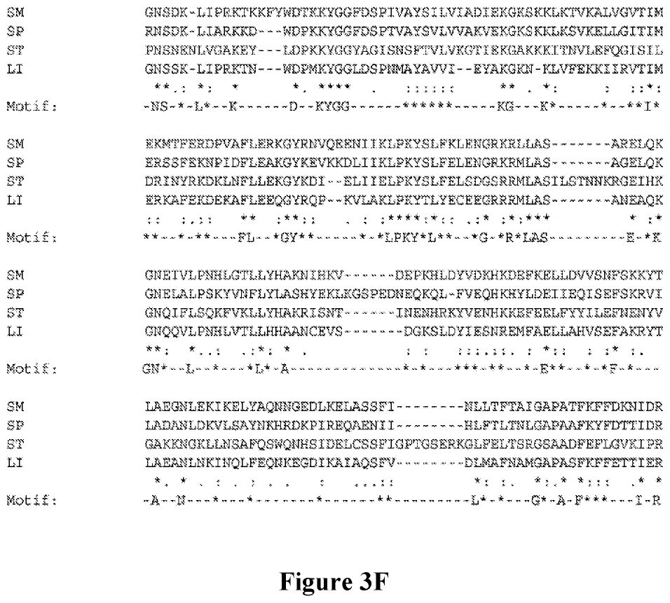

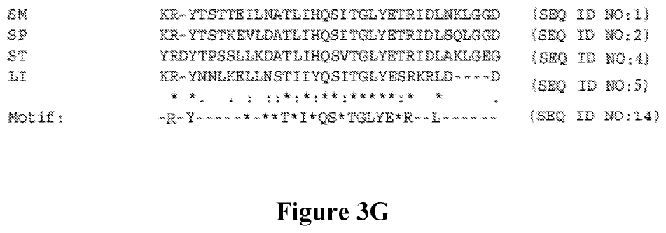

FIGS. 3A-3G depict an alignment of Cas9 sequences (Chylinski 2013). The N-terminal RuvC motif is boxed and indicated with a “Y.” The other two RuvC motifs are boxed and indicated with a “B.” The HNH domain is boxed and indicated by a “G.” Sm: S. mutans (SEQ ID NO:1); Sp: S. pyogenes (SEQ ID NO:2); St: S. thermophilus (SEQ ID NO:4); and Li: L. innocua (SEQ ID NO:5). “Motif” (SEQ ID NO:14) is a consensus sequence based on the four sequences. Residues conserved in all four sequences are indicated by single letter amino acid abbreviation; “*” indicates any amino acid found in the corresponding position of any of the four sequences; and “-” indicates absent.

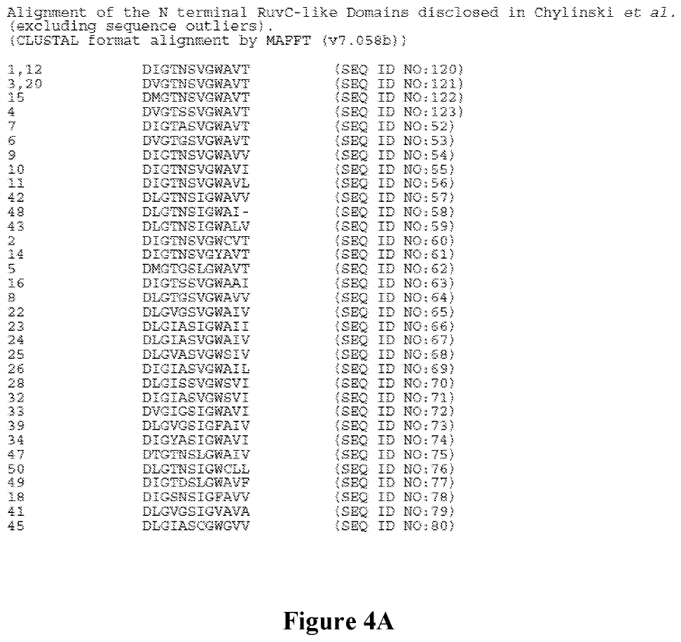

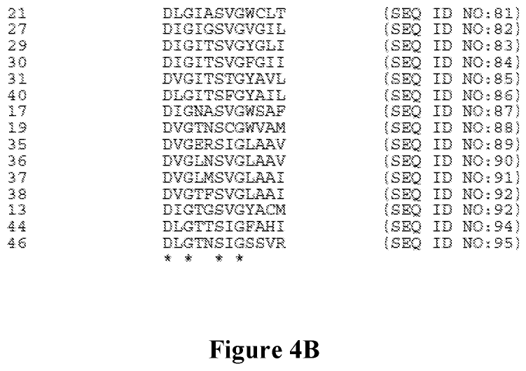

FIGS. 4A-4B show an alignment of the N-terminal RuvC motif from the Cas9 molecules disclosed in Chylinski 2013 with sequence outliers removed (SEQ ID NOs:52-95, 120-123). The last line of FIG. 4B identifies 4 highly conserved residues.

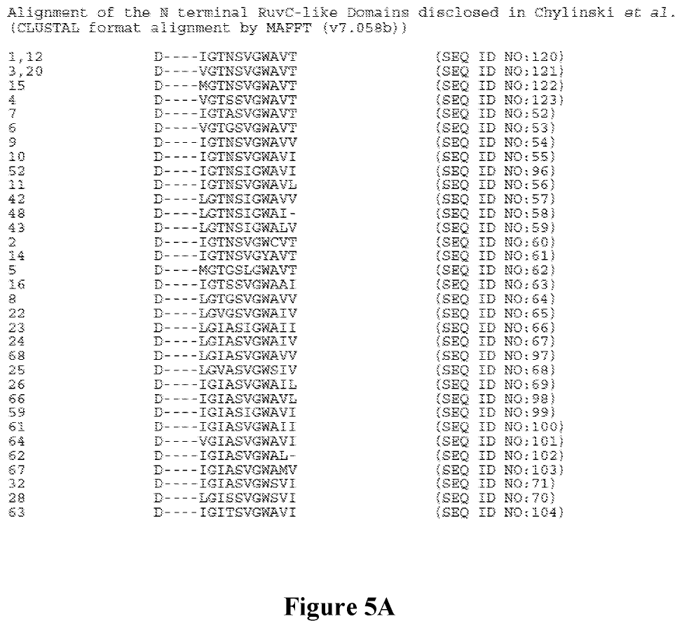

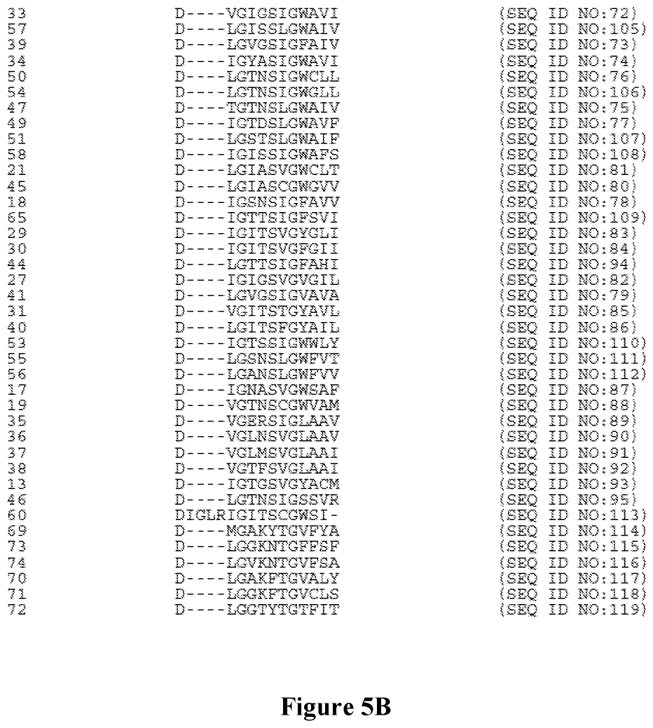

FIGS. 5A-5B show an alignment of the N-terminal RuvC motif from the Cas9 molecules disclosed in Chylinski 2013 (SEQ ID NOs:52-123). The last line of FIG. 5B identifies 3 highly conserved residues.

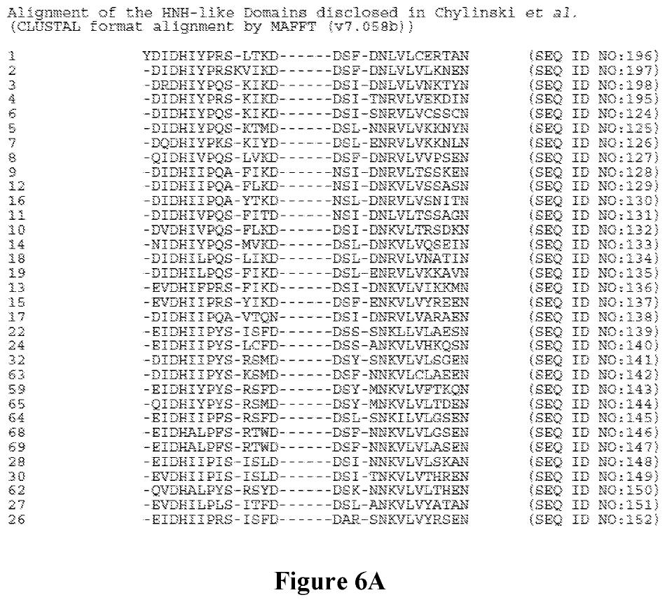

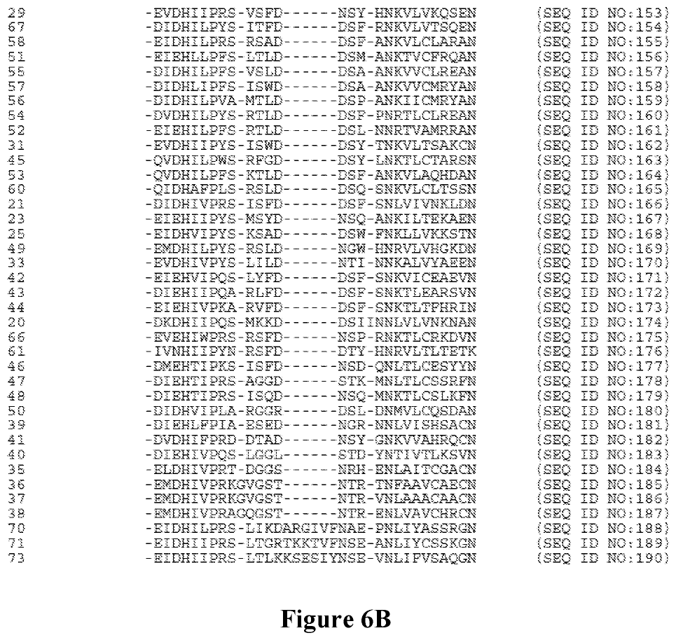

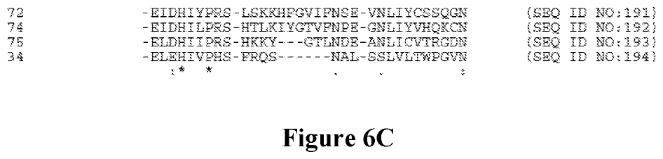

FIGS. 6A-6C show an alignment of the HNH domain from the Cas9 molecules disclosed in Chylinski 2013 (SEQ ID NOs:124-198). The last line of FIG. 6C identifies conserved residues.

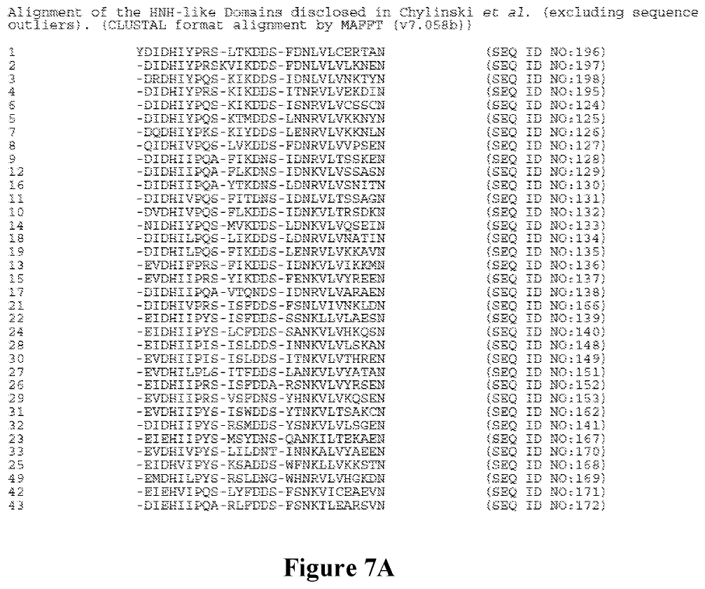

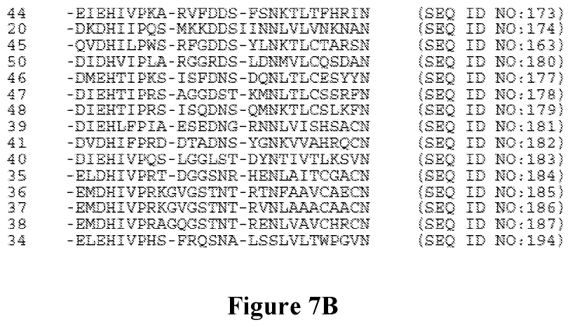

FIGS. 7A-7B show an alignment of the HNH domain from the Cas9 molecules disclosed in Chylinski 2013 with sequence outliers removed (SEQ ID NOs:124-141, 148, 149, 151-153, 162, 163, 166-174, 177-187, 194-198). The last line of FIG. 7B identifies 3 highly conserved residues.

In some embodiments, the target region encodes a lobe of a Cas9 molecule (e.g., the REC lobe or the NUC lobe). In some embodiments, the target region encodes a domain from the REC lobe of a Cas9 molecule (e.g., the BH domain, the REC1 domain, or the REC2 domain). In some embodiments, the target region encodes a motif of a Cas9 molecule (e.g., a REC1 motif at amino acids 94 to 179 or 308 to 717 of the sequence of S. pyogenes Cas9 or the corresponding REC1 motifs in a different Cas9). In some embodiments, the target region encodes a domain from the NUC lobe of a Cas9 molecule (e.g., the RuvC domain, the HNH domain, or the PAM-interacting (PI) domain). In some embodiments, the target region encodes a motif of a Cas9 molecule (e.g., a RuvC I, RuvCII, or RuvCIII motifs at amino acids 1-59, 718-769, and 909-1098, respectively, of the sequence of S. pyogenes Cas9 or the corresponding RuvC I, RuvCII, or RuvCIII motifs in a different Cas9).

### Libraries of Engineered or Altered Cas9 Molecules

In some embodiments, methods and compositions of the present invention can be used to engineer a library of Cas9 molecules and Cas9 polypeptides which possess any of a number of properties, including nuclease activity (e.g., endonuclease and/or exonuclease activity); helicase activity; the ability to associate functionally with a gRNA molecule; and the ability to target (or localize to) a site on a nucleic acid (e.g., PAM recognition and specificity). In certain embodiments, methods and compositions of the present invention can be used to engineer a library of Cas9 molecules or Cas9 polypeptides which include all or a subset of these properties. In a typical embodiment, a Cas9 molecule or Cas9 polypeptide has the ability to interact with a gRNA molecule and, in concert with the gRNA molecule, localize to a site in a nucleic acid. Other activities, e.g., PAM specificity, cleavage activity, or helicase activity can vary more widely in Cas9 molecules and Cas9 polypeptides.

In some embodiments, methods and compositions of the present invention can be used to engineer a library of Cas9 molecules and Cas9 polypeptides which comprise altered enzymatic properties, e.g., altered nuclease activity or altered helicase activity (as compared with a naturally occurring or other reference Cas9 molecule including a Cas9 molecule that has already been engineered or altered). As discussed herein, an engineered Cas9 molecule or Cas9 polypeptide can have nickase activity or no cleavage activity (as opposed to double strand nuclease activity). In an embodiment methods and compositions of the present invention can be used to engineer a library of Cas9 molecules or Cas9 polypeptides which have an alteration that alters its size, e.g., a deletion of amino acid sequence that reduces its size, e.g., with or without significant effect on one or more, or any Cas9 activity. In an embodiment, an engineered Cas9 molecule or Cas9 polypeptide can comprise an alteration that affects PAM recognition (e.g., a Cas9 molecule can be altered to recognize a PAM sequence other than that recognized by the endogenous wild-type PI domain).

Cas9 molecules or Cas9 polypeptides with desired properties can be made in accordance with the present invention in a number of ways, e.g., by alteration of a parental, e.g., naturally occurring, Cas9 molecules or Cas9 polypeptides, to provide an altered Cas9 molecule or Cas9 polypeptide having a desired property. For example, one or more mutations or differences relative to a parental Cas9 molecule, e.g., a naturally occurring or engineered Cas9 molecule, can be introduced. Such mutations and differences comprise: substitutions (e.g., conservative substitutions or substitutions of non-essential amino acids); insertions; or deletions. In an embodiment, a Cas9 molecule or Cas9 polypeptide in a library of the present invention can comprises one or more mutations or differences, e.g., at least 1, 2, 3, 4, 5, 10, 15, 20, 30, 40 or 50 mutations but less than 200, 100, or 80 mutations relative to a reference, e.g., a parental, Cas9 molecule.

### Nucleic Acids Encoding Cas9 Molecules

Nucleic acids encoding Cas9 molecules or Cas9 polypeptides, e.g., an enzymatically active Cas9 (eaCas9) molecule or eaCas9 polypeptides are provided herein. Exemplary nucleic acids encoding Cas9 molecules or Cas9 polypeptides have also been described previously (see, e.g., Cong 2013; Wang 2013; Mali 2013; Jinek 2012).

An exemplary codon optimized nucleic acid sequence encoding a Cas9 molecule of S. pyogenes is set forth in SEQ ID NO:3. The corresponding amino acid sequence of an S. pyogenes Cas9 molecule is set forth in SEQ ID NO:2. Exemplary codon optimized nucleic acid sequences encoding a Cas9 molecule of S. aureus are set forth in SEQ ID NOs:7-9.

Guide RNA (gRNA) Molecules

The terms “guide RNA molecule”, “gRNA molecule” or “gRNA”, are used interchangeably herein, and refer to a nucleic acid that promotes the specific targeting or homing of a gRNA molecule/Cas molecule complex (e.g., a gRNA molecule/Cas9 molecules complex) to a target nucleic acid. gRNA molecules can be unimolecular (having a single RNA molecule) (e.g., chimeric), or modular (comprising more than one, and typically two, separate RNA molecules). The gRNA molecules comprise a targeting domain comprising, consisting of, or consisting essentially of a nucleic acid sequence fully or partially complementary to a target domain. In certain embodiments, the gRNA molecule further comprises one or more additional domains, including for example a first complementarity domain, a linking domain, a second complementarity domain, a proximal domain, a tail domain, and a 5′ extension domain. Each of these domains is discussed below. In certain embodiments, one or more of the domains in the gRNA molecule comprises an amino acid sequence identical to or sharing sequence homology with a naturally occurring sequence, e.g., from S. pyogenes, S. aureus, or S. thermophilus.

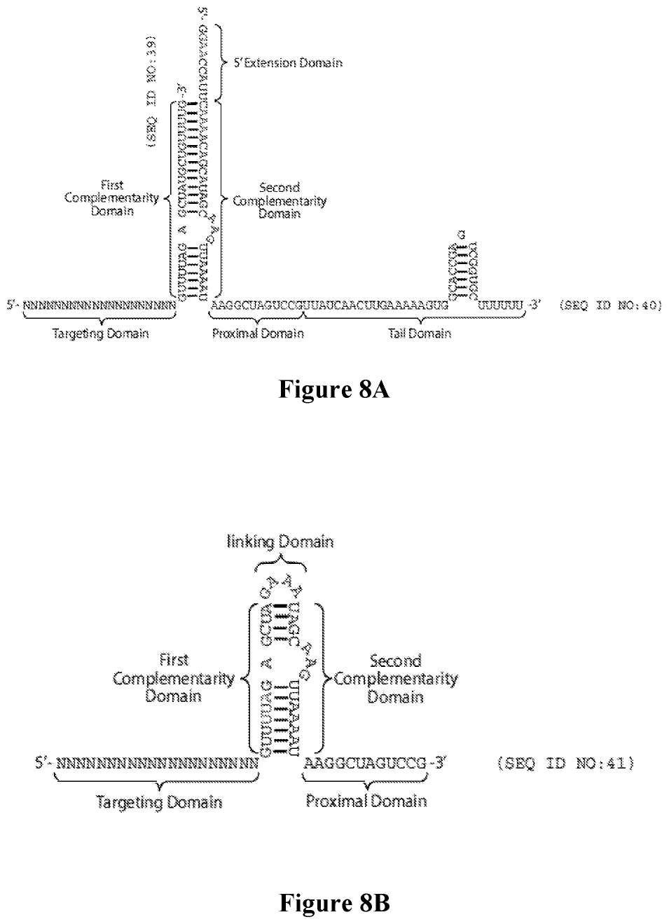

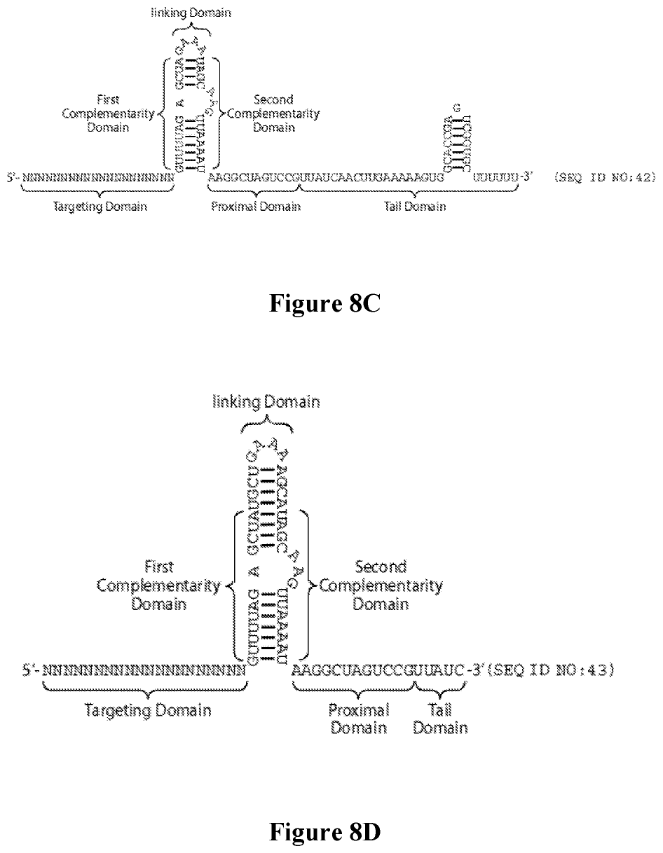

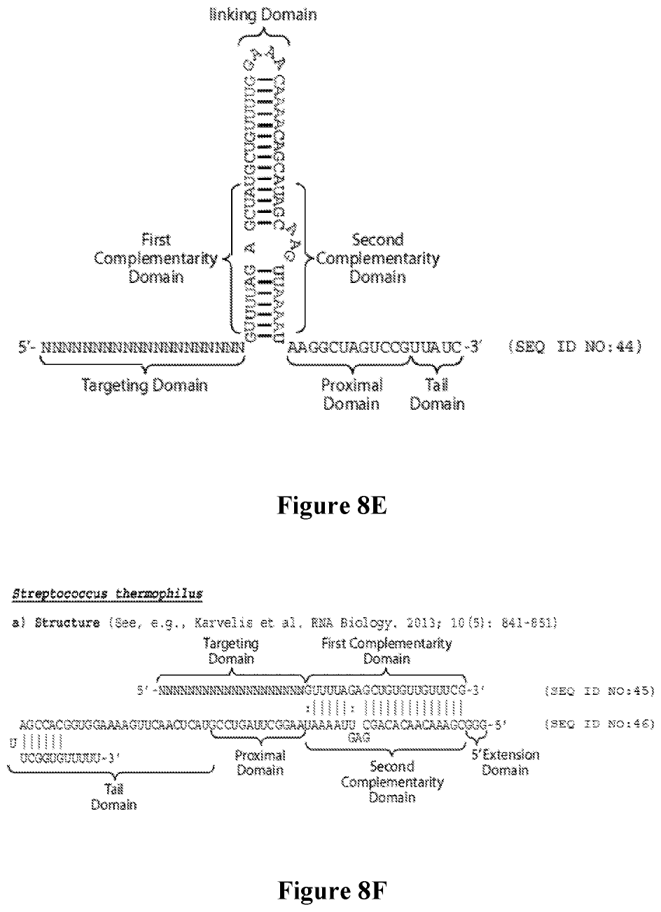

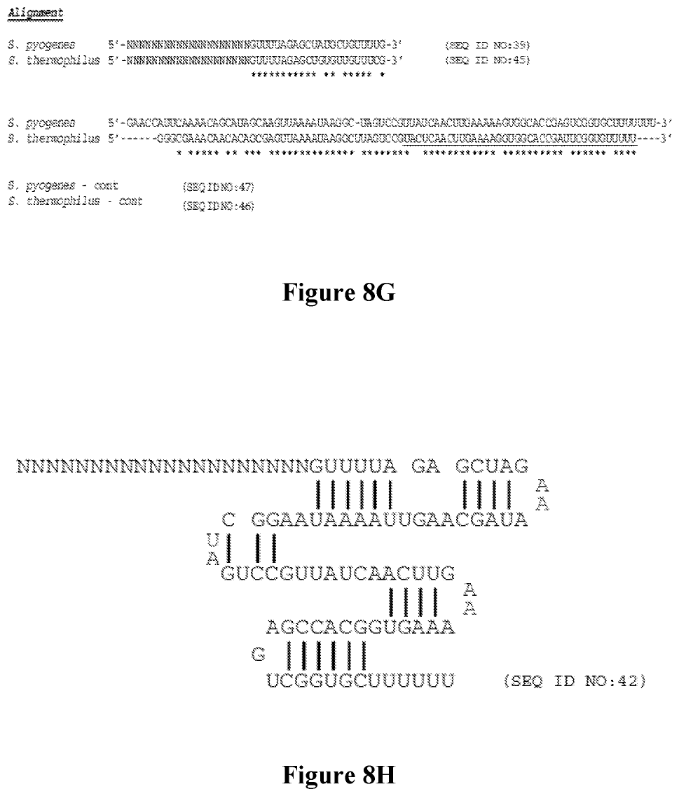

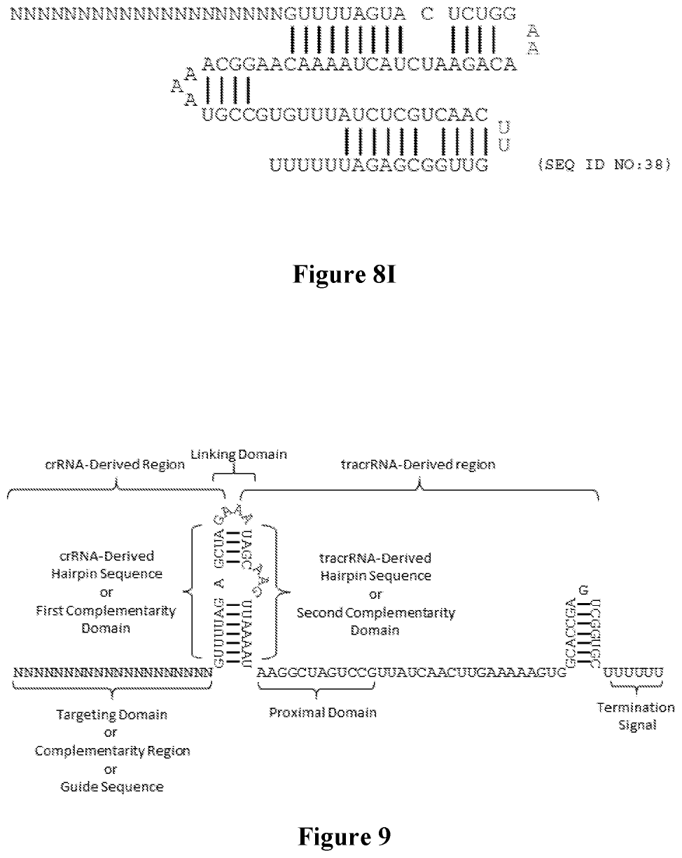

Several exemplary gRNA structures are provided in FIGS. 8A-8I. With regard to the three-dimensional form, or intra- or inter-strand interactions of an active form of a gRNA, regions of high complementarity are sometimes shown as duplexes in FIGS. 8A-8I and other depictions provided herein. FIG. 9 illustrates gRNA domain nomenclature using the gRNA sequence of SEQ ID NO:42, which contains one hairpin loop in the tracrRNA-derived region. In certain embodiments, a gRNA may contain more than one (e.g., two, three, or more) hairpin loops in this region (see, e.g., FIGS. 8H-8I).

In certain embodiments, a unimolecular, or chimeric, gRNA comprises, preferably from 5′ to 3′: a targeting domain; a first complementarity domain; a linking domain; a second complementarity domain (which is complementary to the first complementarity domain); a proximal domain; and optionally, a tail domain.

In certain embodiments, a modular gRNA comprises: a first strand comprising, preferably from 5′ to 3′: a targeting domain; and a first complementarity domain; and a second strand, comprising, preferably from 5′ to 3′: optionally, a 5′ extension domain; a second complementarity domain (which is complementary to the first complementarity domain); a proximal domain; and optionally, a tail domain.

The targeting domain (sometimes referred to alternatively as the guide sequence or complementarity region) comprises, consists of, or consists essentially of a nucleic acid sequence that is complementary or partially complementary to a target nucleic acid sequence in a gene (e.g., a coding region or regulatory region). The nucleic acid sequence in a gene to which all or a portion of the targeting domain is complementary or partially complementary is referred to herein as the target domain. Methods for selecting targeting domains are known in the art (see, e.g., Fu 2014; Sternberg 2014). The strand of the target nucleic acid comprising the target domain is referred to herein as the complementary strand because it is complementary to the targeting domain sequence. Since the targeting domain is part of a gRNA molecule, it comprises the base uracil (U) rather than thymine (T); conversely, any DNA molecule encoding the gRNA molecule will comprise thymine rather than uracil. In a targeting domain/target domain pair, the uracil bases in the targeting domain will pair with the adenine bases in the target domain. In certain embodiments, the degree of complementarity between the targeting domain and target domain is sufficient to allow targeting of a Cas9 molecule to the target nucleic acid.

The first and second complementarity (sometimes referred to alternatively as the crRNA-derived hairpin sequence and tracrRNA-derived hairpin sequences, respectively) domains are fully or partially complementary to one another. In certain embodiments, the degree of complementarity is sufficient for the two domains to form a duplexed region under at least some physiological conditions. In certain embodiments, the degree of complementarity between the first and second complementarity domains, together with other properties of the gRNA, is sufficient to allow targeting of a Cas9 molecule to a target nucleic acid. Examples of first and second complementary domains are set forth in FIGS. 8A-8G.

The linking domain is disposed between and serves to link the first and second complementarity domains in a unimolecular or chimeric gRNA. FIGS. 8B-8E provide examples of linking domains. In certain embodiments, part of the linking domain is from a crRNA-derived region, and another part is from a tracrRNA-derived region. In certain embodiments, the linking domain links the first and second complementarity domains covalently. In certain of these embodiments, the linking domain consists of or comprises a covalent bond. In other embodiments, the linking domain links the first and second complementarity domains non-covalently. In certain embodiments, the linking domain is ten or fewer nucleotides in length, e.g., 1, 2, 3, 4, 5, 6, 7, 8, 9, or 10 nucleotides.

In certain embodiments, a modular gRNA as disclosed herein comprises a 5′ extension domain, i.e., one or more additional nucleotides 5′ to the second complementarity domain (see, e.g., FIG. 8A). In certain embodiments, the 5′ extension domain is 2 to 10 or more, 2 to 9, 2 to 8, 2 to 7, 2 to 6, 2 to 5, or 2 to 4 nucleotides in length. In certain embodiments, the 5′ extension domain has at least 60, 70, 80, 85, 90, or 95% homology with, or differs by no more than 1, 2, 3, 4, 5, or 6 nucleotides from, a reference 5′ extension domain, e.g., a naturally occurring, e.g., an S. pyogenes, S. aureus, or S. thermophilus, 5′ extension domain, or a 5′ extension domain described herein, e.g., from FIGS. 8A-8G.

FIGS. 8A-8G also provide examples of proximal domains. In certain embodiments, the proximal domain is 5 to 20 or more nucleotides in length, e.g., 5, 6, 7, 8, 9, 10, 11, 12, 13, 14, 15, 16, 17, 18, 19, 20, 21, 22, 23, 24, 25, or 26 nucleotides in length. In certain embodiments, the proximal domain can share homology with or be derived from a naturally occurring proximal domain. In certain of these embodiments, the proximal domain has at least 50%, 60%, 70%, 80%, 85%, 90%, or 95% homology with or differs by no more than 1, 2, 3, 4, 5, or 6 nucleotides from a proximal domain disclosed herein, e.g., an S. pyogenes, S. aureus, or S. thermophilus proximal domain, including those set forth in FIGS. 8A-8G.

A broad spectrum of tail domains are suitable for use in the gRNA molecules disclosed herein. FIGS. 8A and 8C-4G provide examples of such tail domains. In certain embodiments, the tail domain is absent. In certain embodiments, the tail domain can share homology with or be derived from a naturally occurring tail domain or the 5′ end of a naturally occurring tail domain. In certain of these embodiments, the proximal domain has at least 50%, 60%, 70%, 80%, 85%, 90%, or 95% homology with or differs by no more than 1, 2, 3, 4, 5, or 6 nucleotides from a naturally occurring tail domain disclosed herein, e.g., an S. pyogenes, S. aureus, or S. thermophilus tail domain, including those set forth in FIGS. 8A and 8C-8G.

### REFERENCES

Anders et al. Nature 513(7519):569-573 (2014)
Barnes Gene 112:29-35 (1992)
Cong et al. Science 399(6121):819-823 (2013)
Chylinski et al. RNA Biol 10(5):726-737 (2013)
Fu et al. Nat Biotechnol 32:279-284 (2014)
Haft et al. PLoS Computational Biology 1(6): e60 (2005)
Jinek et al. Science 337(6096):816-821 (2012)
Jinek et al. Science 343(6176):1247997 (2014)
Kunkel and Tindall Biochemistry 27:6008-6013 (1988)
Makarova et al. Nature Review Microbiology 9:467-477 (2011)
Makarova et al. Nature Reviews Microbiology 13:722-736 (2015)
Mali et al. Science 399(6121):823-826 (2013)
Nishimasu et al. Cell 156(5):935-949 (2014)
Quan and Tiang PLoS ONE 4(7): e6441 (2009)
Shmakov et al. Mol Cell 60(3):385-97 (2015)
Sternberg et al. Nature 507(7490):62-67 (2014)
Wang et al. Cell 153(4):910-918 (2013)
Zetsche et al. Cell 163:759-771 (2015)

### EXAMPLES

### Example 1: Library Generation for a Cas9 Polypeptide

Random codon mutation of a Cas9 polypeptide allows for a diverse and unbiased library of variants. In order to create a library, we designed 1054 degenerate oligonucleotides targeting every codon of a Cas9 polypeptide. Oligonucleotide primers were designed via a custom Matlab program that optimized primers for length, Tm, GC %, secondary structure, end stability, repeats, and dimers. Each synthesized primer contained a degenerate nucleotide triplet (“NNN”), corresponding to the codon to be randomly mutated. This degenerate site was flanked by DNA homologous to the flanking sites of the codon to be mutated. Additionally, phosphorylated non-degenerate reverse primer (“T7-term”) and an abutting forward primer (“T7-F”), were synthesized.

Plasmid containing the Cas9 sequence was isolated. In parallel, degenerate oligonucleotide primers were pooled at equimolar concentration and phosphorylated in a polynucleotide kinase reaction. The phosphorylated oligo pool was mixed with 100 ng template plasmid at 100-fold molar excess along with 100-fold molar excess of non-degenerate reverse primer and optionally equimolar non-degenerate, non-mutagenic forward primer. Phusion® Mastermix (New England Biolabs) and water were added to a total volume of 50 μl. The reaction was cycled at 98° C. for 30 seconds; 25 cycles of 98° C. for 30 seconds, 55° C. for 30 seconds, and 72° C. for 10 minutes; and a final extension for 10 minutes at 72° C. 1 μl each of DpnI, Lambda Exonuclease, and Exo I were added to the reaction mixture and incubated at 37° C. for 60 minutes. After incubation, the reaction was purified and electroporated into NEB® Turbo electrocompetent E. coli (New England Biolabs). The transformed E. coli were plated on selective media and grown overnight at 37° C.

We observed roughly 10⁶ surviving colony-forming units. Surviving colonies were pooled and plasmids were isolated from the library. The Cas9 region of plasmids from the library was then amplified via PCR and subjected to PacBio sequencing. Custom Matlab scripts were used to verify sites of mutations found on each read.

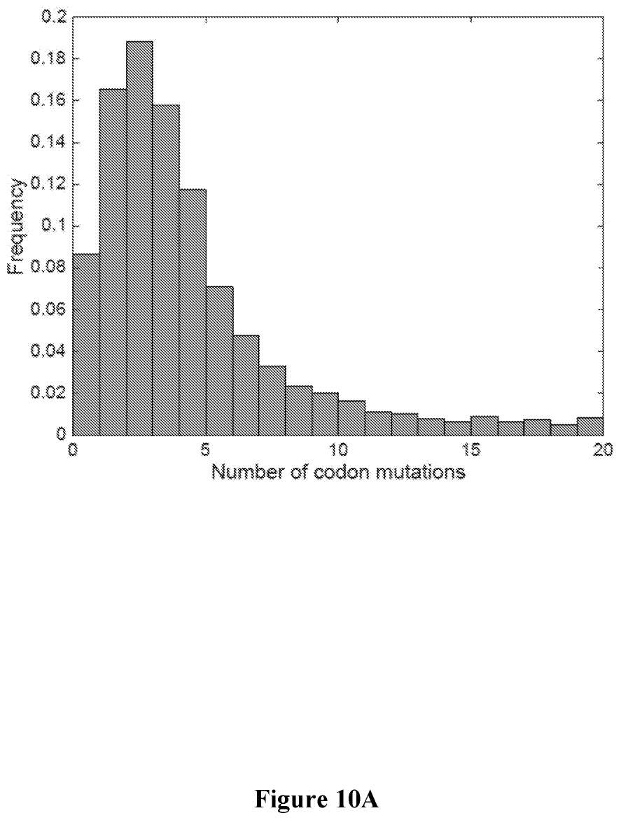

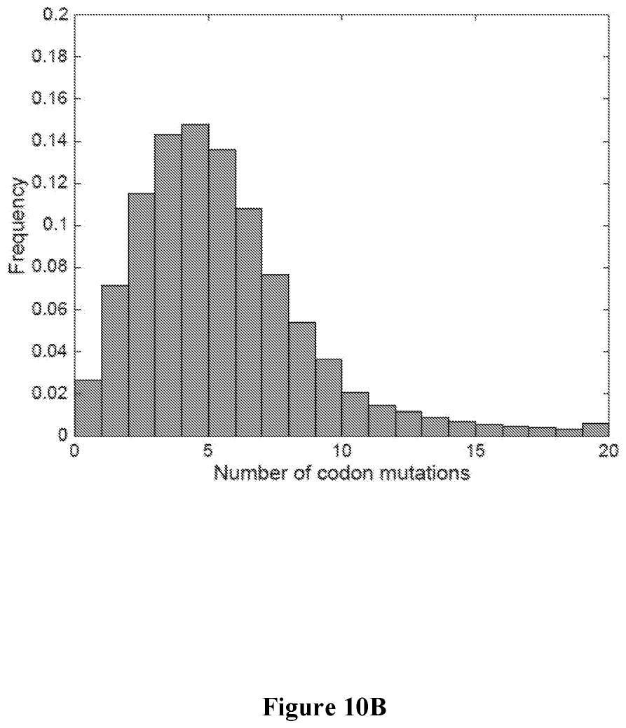

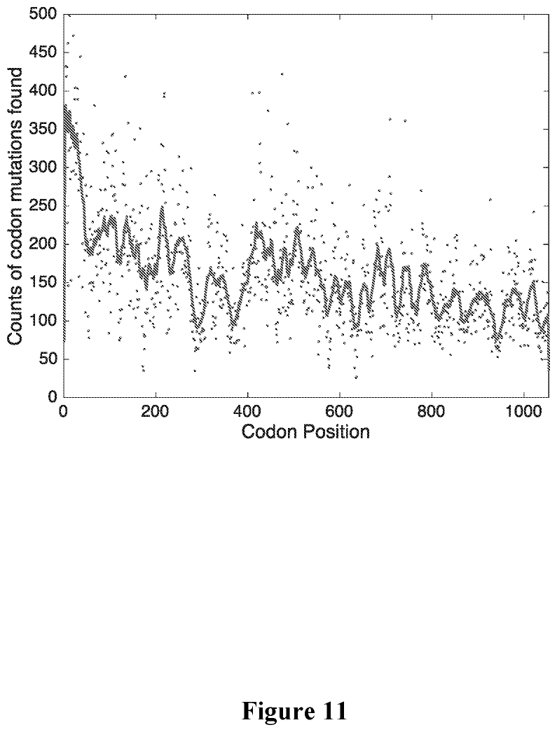

Results from next-generation sequencing demonstrated that the mutation rate was tunable. As shown in FIGS. 10A-10B, when the reaction was spiked with a non-degenerate, non-mutagenic forward primer, the median number of codon mutations was about three (FIG. 5A). On the other hand, the median number of codon mutations in un-spiked reactions was about five (FIG. 10B).

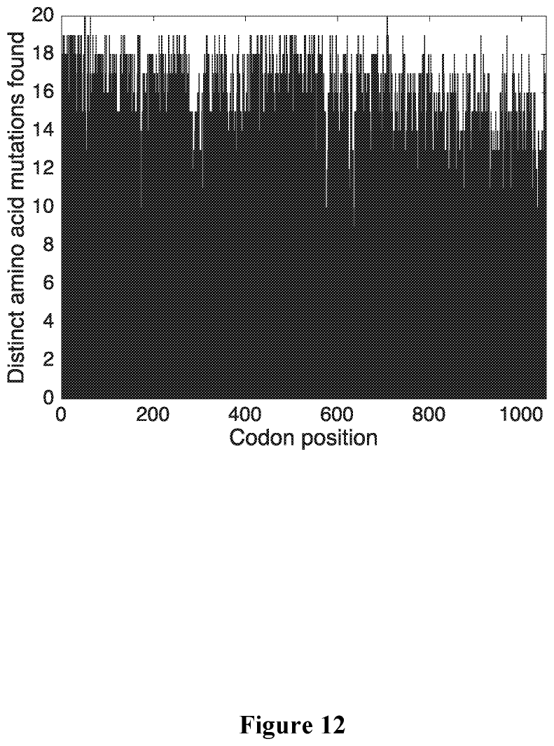

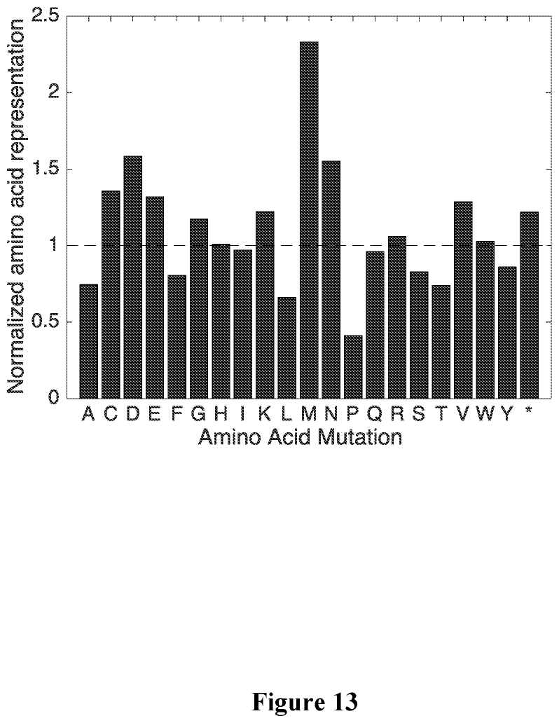

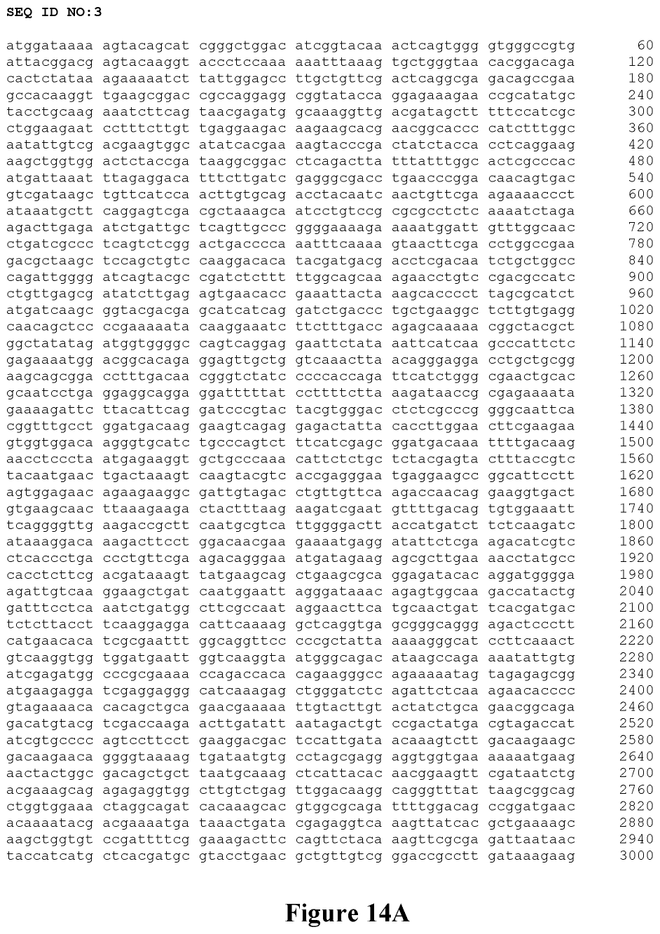

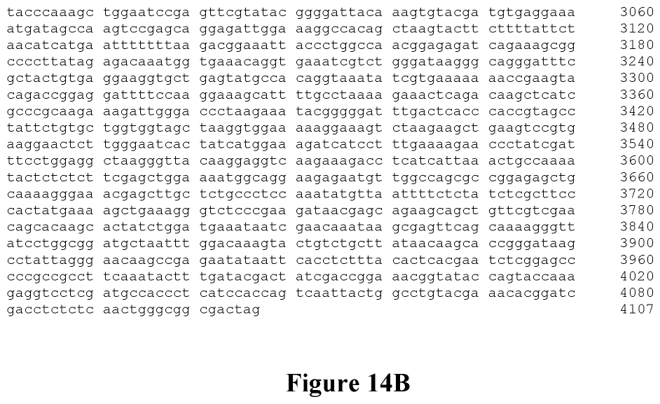

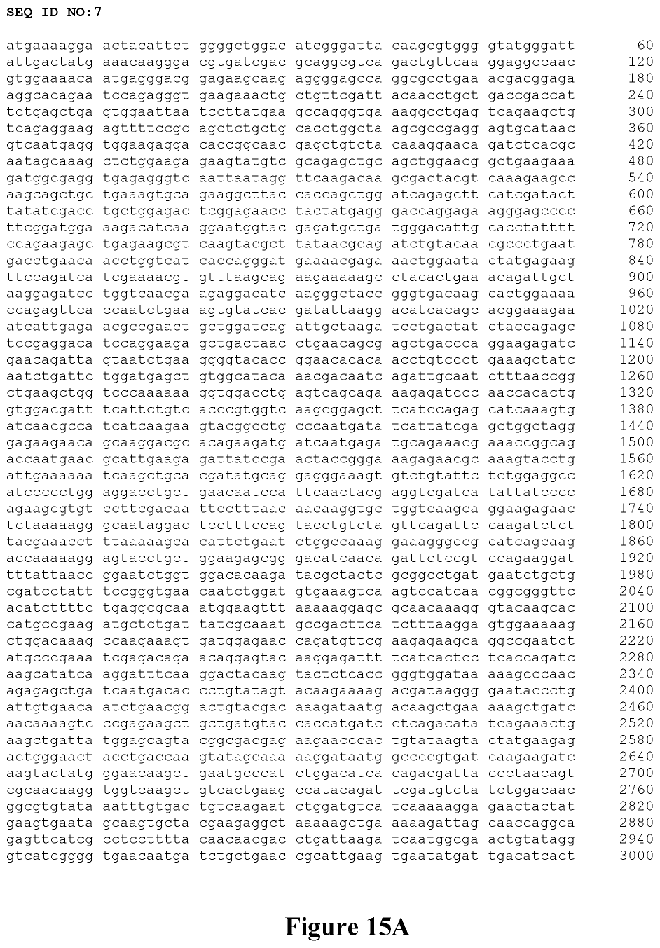

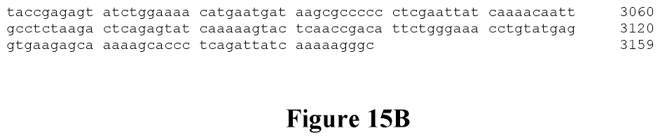

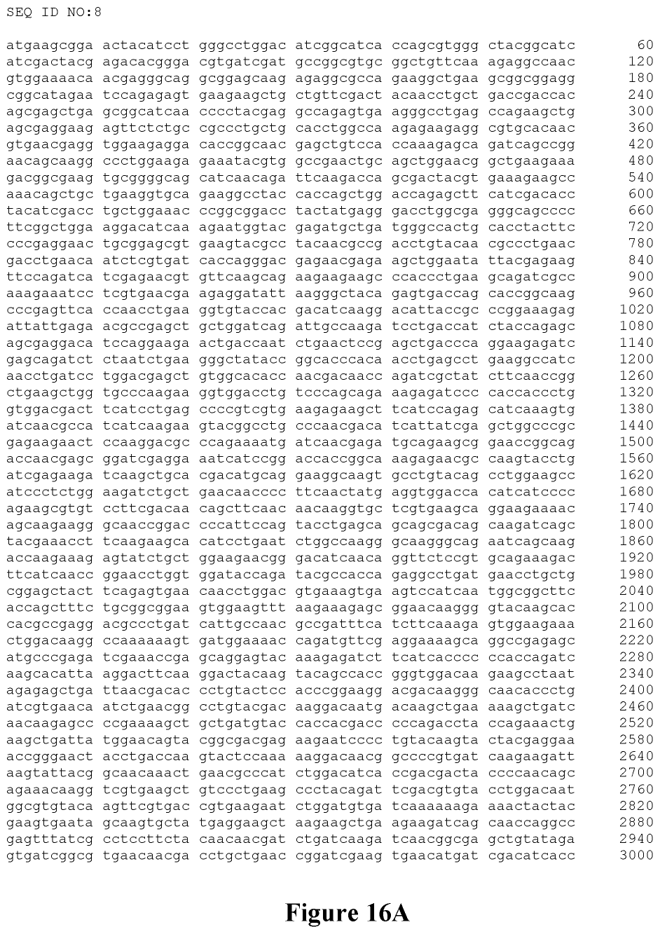

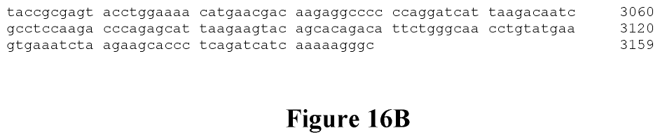

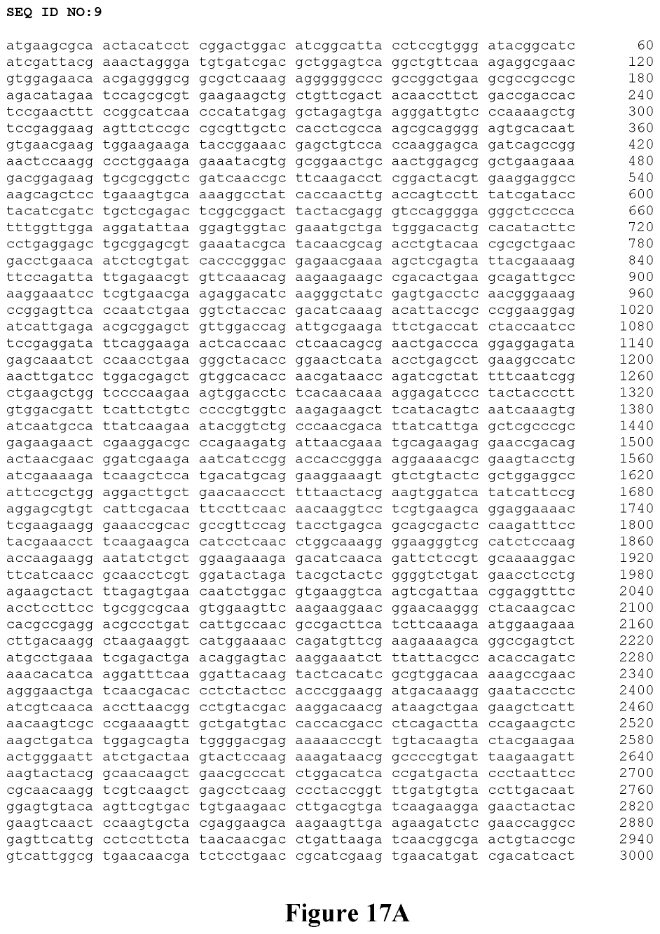

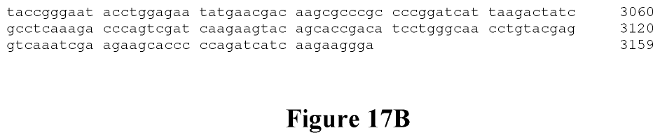

Additionally, there was a low overall position bias (FIG. 11), low overall codon bias (FIG. 12), and low overall bias among amino acids represented in mutations (FIG. 13).

### Example 2: Library Generation for an RNA Aptamer

An aptamer library is created using the same approach as Example 1 to create highly diverse RNA sequences from a DNA template that encodes an RNA aptamer. Libraries of mutant copies with targeted base-pair mutations or randomized “N” degenerate base-pair mutations are engineered by designing oligonucleotides with the mutagenic base flanked by homologous DNA to the template. The reaction randomly incorporates these designated mutations to create DNA encoding the aptamer library.

### Example 3: Library Generation for a DNA Binding Site

Transcription factor binding sites are empirically tuned to different activities through mutagenic libraries. DNA libraries are created using the same approach as Example 1 to create highly diverse DNA sequences from a DNA template that encodes a transcription factor. Libraries of mutant copies with targeted base-pair mutations or randomized “N” degenerate base-pair mutations within the transcription factor binding site are created by designing oligonucleotides with the mutagenic base flanked by DNA homologous to the template. The reaction randomly incorporates these designated mutations to create DNA encoding the binding site library.

### Example 4: Library Generation for Antibodies

Random codon mutation of antibodies allows for an extremely diverse and unbiased library of antibody variants. Degenerate or designated mutagenic primers are used to comprehensively mutate the coding region of the antibody or to target suspected binding regions (e.g., CDRs). Each synthesized primer contains a degenerate nucleotide triplet (“NNN”), corresponding to the codon to be randomly mutated. This degenerate site is flanked by DNA homologous to the flanking sites of the codon to be mutated. The reaction is performed using the same approach as Example 1 and randomly incorporates these designated mutations to create DNA encoding the antibody library.

### Example 5: Library Generation of Silent Mutations

Codon usage is often un-optimized in expression constructs. Random codon mutation allows for an extremely diverse and unbiased library of variants. Designated “silent” mutagenic primers can mutate part or all of the coding region of the protein. Each synthesized primer contains one or more mutagenic codons corresponding to a synonymous codon to the template. This mutagenic site is flanked by DNA homologous to the flanking sites of the codon to be mutated. The reaction is performed using the same approach as Example 1 and randomly incorporates these designated mutations to create DNA with the same amino acid sequence but with a distinct codon usage set.

### Example 6: Insertion or Deletion Library for a Cas9 gRNA Sequence

Sequences of gRNA molecules can be randomly lengthened or deleted using methods of the present invention. Deletions (the lack of one or multiple base-pairs flanked by homologous DNA) or insertions (the addition of extra DNA flanked by homologous DNA) may be designed into the primers and targeted to specified regions of DNA encoding a gRNA for a particular Cas9. The reaction is performed using the same approach as Example 1 and randomly incorporates these insertions or deletions into the gRNA DNA sequence, creating a library of varying length.

1. A method of preparing mutant copies of a double-stranded template polynucleotide, the method comprising steps of:
(a) providing a double-stranded template polynucleotide that comprises a first strand and a second strand, wherein the double-stranded template polynucleotide comprises a target region;
(b) providing a pool of first oligonucleotide primers, wherein each of the first oligonucleotide primers is independently
(i) capable of hybridizing to the first strand within the target region, and
(ii) complementary to a sequence within the target region except for at least one mutagenic site where the first oligonucleotide primer and first strand are non-complementary;
(c) providing a second oligonucleotide primer that is capable of hybridizing to the second strand; and
(d) combining the double-stranded template polynucleotide, pool of first oligonucleotide primers, and second oligonucleotide primer under conditions that allow amplification of the double-stranded template polynucleotide,
thereby generating mutant copies of the double-stranded template polynucleotide, wherein each mutant copy includes a mutated version of at least a portion of the target region.

2. The method of claim 1, wherein the target region encodes a polypeptide and comprises a plurality of codons, and wherein the at least one mutagenic site includes 1, 2 or all 3 nucleotides within one of the codons.

3. The method of claim 1, wherein the pool of first oligonucleotide primers collectively span the entire length of the target region.

4-7. (canceled)

8. The method of claim 1, wherein the second oligonucleotide primer is capable of hybridizing to the second strand outside the target region and is not capable of hybridizing to the second strand in the target region.

9. The method of claim 1, wherein the double-stranded template polynucleotide is circular.

10. (canceled)

11. The method of claim 1, further comprising a step of providing a third oligonucleotide primer which that is 100% complementary to the first strand of the double-stranded template polynucleotide, and wherein step (d) comprises combining the third oligonucleotide primer together with the double-stranded template polynucleotide, pool of first oligonucleotide primers, and second oligonucleotide primer under conditions that allow amplification of the double-stranded template polynucleotide.

12. The method of claim 11, wherein the third oligonucleotide is 100% complementary to the first strand of the double-stranded template polynucleotide outside of the target region.

13. (canceled)

14. The method of claim 1, wherein the conditions comprise incubating the double-stranded template polynucleotide, the pool of first oligonucleotide primers, and the second oligonucleotide primer together in a reaction mixture.

15. (canceled)

16. The method of claim 14, wherein the reaction mixture comprises a DNA polymerase.

17-19. (canceled)

20. The method of claim 16, wherein the DNA polymerase has an error rate of less than three base pair changes per kilobase of DNA.

21-25. (canceled)

26. The method of claim 13, further comprising a step of:
(e) combining the reaction mixture with one or more nucleases under conditions to allow activity of the one or more nucleases.

27. The method of claim 26, wherein the one or more nucleases comprises a methylation-specific nuclease, an exonuclease, a nuclease specific for single-stranded DNA, or a combination thereof.

28. (canceled)

29. The method of claim 27, wherein the methylation-specific nuclease is DpnI.

30-32. (canceled)

33. The method of claim 27, wherein the nuclease specific for single-stranded DNA is Exonuclease I (ExoI).

34. The method of claim 1, wherein the molar ratio of the pool of first oligonucleotide primers to the double-stranded template polynucleotide is 1:1 or greater.

35-36. (canceled)

37. The method of claim 1, wherein the molar ratio of the second oligonucleotide primer to the double-stranded template polynucleotide is 1:1 or greater.

38-39. (canceled)

40. The method of claim 1, further comprising a step of transforming cells with the library of mutated polynucleotides into cells.

41. A library obtained by the method of claim 1.


SEQUENCE LISTING (198 sequences)

SEQ ID NO: 1 - protein, 1345 aa, Streptococcus mutans
  REGION 10..21: note = MISC_FEATURE - N-terminal RuvC-like domain
  REGION 759..766: note = MISC_FEATURE - RuvC-like domain
  REGION 837..863: note = MISC_FEATURE - HNH-like domain
  REGION 982..989: note = MISC_FEATURE - RuvC-like domain
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 2 - protein, 1368 aa, Streptococcus pyogenes [sequence omitted]

SEQ ID NO: 3 - DNA, 4107 bp, Streptococcus pyogenes
  source 1..4107: mol_type = unassigned DNA
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 4 - protein, 1388 aa, Streptococcus thermophilus
  REGION 10..21: note = MISC_FEATURE - N-terminal RuvC-like domain
  REGION 760..767: note = MISC_FEATURE - RuvC-like domain
  REGION 844..870: note = MISC_FEATURE - HNH-like domain
  REGION 989..996: note = MISC_FEATURE - RuvC-like domain
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 5 - protein, 1334 aa, Listeria innocua
  REGION 10..21: note = MISC_FEATURE - N-terminal RuvC-like domain
  REGION 762..769: note = MISC_FEATURE - RuvC-like domain
  REGION 840..866: note = MISC_FEATURE - HNH-like domain
  REGION 985..992: note = MISC_FEATURE - RuvC-like domain
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 6 - skipped

SEQ ID NO: 7 - DNA, 3159 bp, Staphylococcus aureus
  source 1..3159: mol_type = unassigned DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 8 - DNA, 3159 bp, Staphylococcus aureus
  source 1..3159: mol_type = unassigned DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 9 - DNA, 3159 bp, Staphylococcus aureus
  source 1..3159: mol_type = unassigned DNA
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 10-13 - skipped (4 entries)

SEQ ID NO: 14 - protein, 1417 aa, synthetic construct
  VARIANT 2: note = MOD_RES - Any amino acid or absent
  VARIANT 4: note = MOD_RES - Any amino acid or absent
  VARIANT 6: note = Can be replaced by Thr
  REGION 8..18: note = MISC_FEATURE - N-terminal RuvC-like domain
  VARIANT 21: note = MOD_RES - Any amino acid or absent
  VARIANT 24: note = Can be replaced by Glu or Asn or Gln
  VARIANT 26: note = MOD_RES - Any amino acid or absent
  VARIANT 27: note = Can be replaced by Leu
  VARIANT 28: note = MOD_RES - Any amino acid or absent
  VARIANT 29: note = MOD_RES - Any amino acid or absent
  VARIANT 30: note = Can be replaced by Arg
  VARIANT 32: note = Can be replaced by Phe
  VARIANT 34: note = Can be replaced by Ile
  VARIANT 35: note = MOD_RES - Any amino acid or absent
  VARIANT 37: note = Can be replaced by Asp
  VARIANT 38: note = Can be replaced by Ser
  VARIANT 39: note = MOD_RES - Any amino acid or absent
  VARIANT 40: note = Can be replaced by Arg
  VARIANT 41: note = MOD_RES - Any amino acid or absent
  VARIANT 42: note = MOD_RES - Any amino acid or absent
  VARIANT 44: note = Can be replaced by Lys
  VARIANT 47: note = Can be replaced by Phe
  VARIANT 48: note = MOD_RES - Any amino acid or absent
  VARIANT 50: note = MOD_RES - Any amino acid or absent
  VARIANT 51: note = MOD_RES - Any amino acid or absent
  VARIANT 55: note = MOD_RES - Any amino acid or absent
  VARIANT 57: note = MOD_RES - Any amino acid or absent
  VARIANT 60: note = MOD_RES - Any amino acid or absent
  VARIANT 61: note = MOD_RES - Any amino acid or absent
  VARIANT 62: note = MOD_RES - Any amino acid or absent
  VARIANT 64: note = Can be replaced by Met
  VARIANT 65: note = MOD_RES - Any amino acid or absent
  VARIANT 72: note = MOD_RES - Any amino acid or absent
  VARIANT 73: note = MOD_RES - Any amino acid or absent
  VARIANT 76: note = Can be replaced by Lys
  VARIANT 80: note = MOD_RES - Any amino acid or absent
  VARIANT 84: note = MOD_RES - Any amino acid or absent
  VARIANT 87: note = Can be replaced by Ala
  VARIANT 88: note = MOD_RES - Any amino acid or absent
  VARIANT 91: note = MOD_RES - Any amino acid or absent
  VARIANT 92: note = MOD_RES - Any amino acid or absent
  VARIANT 93: note = MOD_RES - Any amino acid or absent
  VARIANT 95: note = MOD_RES - Any amino acid or absent
  VARIANT 96: note = MOD_RES - Any amino acid or absent
  VARIANT 99: note = MOD_RES - Any amino acid or absent
  VARIANT 102: note = MOD_RES - Any amino acid or absent
  VARIANT 103: note = Can be replaced by Glu
  VARIANT 106: note = MOD_RES - Any amino acid or absent
  VARIANT 108: note = MOD_RES - Any amino acid or absent
  VARIANT 109: note = Can be replaced by Asp or Asn
  VARIANT 110: note = Can be replaced by Glu
  VARIANT 112: note = Can be replaced by Lys
  VARIANT 113: note = MOD_RES - Any amino acid or absent
  VARIANT 114: note = MOD_RES - Any amino acid or absent
  VARIANT 115: note = Can be replaced by Lys
  VARIANT 116: note = Can be replaced by Tyr
  VARIANT 118: note = Can be replaced by Phe
  VARIANT 120: note = MOD_RES - Any amino acid or absent
  VARIANT 121: note = MOD_RES - Any amino acid or absent
  VARIANT 122: note = Can be replaced by Ile
  VARIANT 123: note = MOD_RES - Any amino acid or absent
  VARIANT 124: note = Can be replaced by Asp
  VARIANT 126: note = MOD_RES - Any amino acid or absent
  VARIANT 127: note = MOD_RES - Any amino acid or absent
  VARIANT 130: note = MOD_RES - Any amino acid or absent
  VARIANT 131: note = Can be replaced by Lys or Glu
  VARIANT 132: note = Can be replaced by Tyr
  VARIANT 140: note = Can be replaced by Lys or Glu
  VARIANT 141: note = MOD_RES - Any amino acid or absent
  VARIANT 143: note = MOD_RES - Any amino acid or absent
  VARIANT 144: note = Can be replaced by Asn
  VARIANT 145: note = MOD_RES - Any amino acid or absent
  VARIANT 146: note = MOD_RES - Any amino acid or absent
  VARIANT 147: note = MOD_RES - Any amino acid or absent
  VARIANT 149: note = MOD_RES - Any amino acid or absent
  VARIANT 154: note = Can be replaced by Ile
  VARIANT 161: note = Can be replaced by Met
  VARIANT 164: note = Can be replaced by Tyr
  VARIANT 173: note = MOD_RES - Any amino acid or absent
  VARIANT 174: note = Can be replaced by Leu
  VARIANT 175: note = Can be replaced by Asn
  VARIANT 176: note = MOD_RES - Any amino acid or absent
  VARIANT 177: note = MOD_RES - Any amino acid or absent
  VARIANT 179: note = MOD_RES - Any amino acid or absent
  VARIANT 180: note = MOD_RES - Any amino acid or absent
  VARIANT 181: note = Can be replaced by Ile
  VARIANT 182: note = Can be replaced by Asp
  VARIANT 183: note = MOD_RES - Any amino acid or absent
  VARIANT 184: note = MOD_RES - Any amino acid or absent
  VARIANT 185: note = Can be replaced by Tyr
  VARIANT 186: note = MOD_RES - Any amino acid or absent
  VARIANT 187: note = Can be replaced by Gln or Asp
  VARIANT 188: note = Can be replaced by Leu
  VARIANT 189: note = Can be replaced by Val or Ile
  VARIANT 190: note = MOD_RES - Any amino acid or absent
  VARIANT 191: note = MOD_RES - Any amino acid or absent
  VARIANT 193: note = Can be replaced by Asn
  VARIANT 194: note = MOD_RES - Any amino acid or absent
  VARIANT 195: note = MOD_RES - Any amino acid or absent
  VARIANT 197: note = MOD_RES - Any amino acid or absent
  VARIANT 198: note = MOD_RES - Any amino acid or absent
  VARIANT 199: note = MOD_RES - Any amino acid or absent
  VARIANT 200: note = MOD_RES - Any amino acid or absent
  VARIANT 201: note = MOD_RES - Any amino acid or absent
  VARIANT 202: note = MOD_RES - Any amino acid or absent
  VARIANT 203: note = MOD_RES - Any amino acid or absent
  VARIANT 204: note = MOD_RES - Any amino acid or absent
  VARIANT 205: note = MOD_RES - Any amino acid or absent
  VARIANT 206: note = MOD_RES - Any amino acid or absent
  VARIANT 207: note = MOD_RES - Any amino acid or absent
  VARIANT 208: note = MOD_RES - Any amino acid or absent
  VARIANT 209: note = MOD_RES - Any amino acid or absent
  VARIANT 210: note = MOD_RES - Any amino acid or absent
  VARIANT 211: note = MOD_RES - Any amino acid or absent
  VARIANT 212: note = MOD_RES - Any amino acid or absent
  VARIANT 213: note = Can be replaced by Asp
  VARIANT 214: note = MOD_RES - Any amino acid or absent
  VARIANT 215: note = MOD_RES - Any amino acid or absent
  VARIANT 216: note = MOD_RES - Any amino acid or absent
  VARIANT 218: note = Can be replaced by Val
  VARIANT 219: note = MOD_RES - Any amino acid or absent
  VARIANT 220: note = MOD_RES - Any amino acid or absent
  VARIANT 221: note = Can be replaced by Arg
  VARIANT 222: note = Can be replaced by Leu or Val
  VARIANT 223: note = Can be replaced by Thr
  VARIANT 224: note = Can be replaced by Arg
  VARIANT 225: note = MOD_RES - Any amino acid or absent
  VARIANT 226: note = MOD_RES - Any amino acid or absent
  VARIANT 227: note = Can be replaced by Arg
  VARIANT 228: note = MOD_RES - Any amino acid or absent
  VARIANT 229: note = Can be replaced by Glu
  VARIANT 230: note = MOD_RES - Any amino acid or absent
  VARIANT 231: note = Can be replaced by Leu or Ile
  VARIANT 232: note = Can be replaced by Ile
  VARIANT 233: note = MOD_RES - Any amino acid or absent
  VARIANT 234: note = MOD_RES - Any amino acid or absent
  VARIANT 235: note = MOD_RES - Any amino acid or absent
  VARIANT 237: note = MOD_RES - Any amino acid or absent
  VARIANT 240: note = MOD_RES - Any amino acid or absent
  VARIANT 241: note = MOD_RES - Any amino acid or absent
  VARIANT 243: note = MOD_RES - Any amino acid or absent
  VARIANT 245: note = MOD_RES - Any amino acid or absent
  VARIANT 246: note = Can be replaced by Asn or Gln
  VARIANT 247: note = Can be replaced by Leu
  VARIANT 248: note = Can be replaced by Ile
  VARIANT 249: note = MOD_RES - Any amino acid or absent
  VARIANT 251: note = MOD_RES - Any amino acid or absent
  VARIANT 252: note = Can be replaced by Leu
  VARIANT 254: note = MOD_RES - Any amino acid or absent
  VARIANT 255: note = MOD_RES - Any amino acid or absent
  VARIANT 256: note = MOD_RES - Any amino acid or absent
  VARIANT 257: note = Can be replaced by Asn
  VARIANT 259: note = Can be replaced by Arg or Gln
  VARIANT 260: note = MOD_RES - Any amino acid or absent
  VARIANT 261: note = MOD_RES - Any amino acid or absent
  VARIANT 263: note = Can be replaced by Asp or Asn
  VARIANT 265: note = MOD_RES - Any amino acid or absent
  VARIANT 267: note = MOD_RES - Any amino acid or absent
  VARIANT 268: note = Can be replaced by Ser
  VARIANT 269: note = MOD_RES - Any amino acid or absent
  VARIANT 270: note = Can be replaced by Ile
  VARIANT 271: note = MOD_RES - Any amino acid or absent
  VARIANT 272: note = MOD_RES - Any amino acid or absent
  VARIANT 273: note = Can be replaced by Ala
  VARIANT 275: note = Can be replaced by Glu
  VARIANT 276: note = Can be replaced by Ser
  VARIANT 278: note = Can be replaced by Asp
  VARIANT 279: note = Can be replaced by Asp
  VARIANT 280: note = Can be replaced by Asp
  VARIANT 282: note = Can be replaced by Asp
  VARIANT 283: note = MOD_RES - Any amino acid or absent
  VARIANT 286: note = MOD_RES - Any amino acid or absent
  VARIANT 287: note = MOD_RES - Any amino acid or absent
  VARIANT 291: note = Can be replaced by Gln or Asp or Glu
  VARIANT 293: note = Can be replaced by Ser
  VARIANT 294: note = Can be replaced by Asp
  VARIANT 295: note = Can be replaced by Val
  VARIANT 297: note = Can be replaced by Val
  VARIANT 298: note = MOD_RES - Any amino acid or absent
  VARIANT 301: note = Can be replaced by Asn
  VARIANT 302: note = MOD_RES - Any amino acid or absent
  VARIANT 303: note = MOD_RES - Any amino acid or absent
  VARIANT 304: note = MOD_RES - Any amino acid or absent
  VARIANT 305: note = Can be replaced by Ala
  VARIANT 306: note = Can be replaced by Val
  VARIANT 307: note = Can be replaced by Val
  VARIANT 310: note = MOD_RES - Any amino acid or absent
  VARIANT 311: note = Can be replaced by Phe
  VARIANT 312: note = Can be replaced by Ile
  VARIANT 313: note = MOD_RES - Any amino acid or absent
  VARIANT 315: note = MOD_RES - Any amino acid or absent
  VARIANT 316: note = MOD_RES - Any amino acid or absent
  VARIANT 317: note = MOD_RES - Any amino acid or absent
  VARIANT 318: note = MOD_RES - Any amino acid or absent
  VARIANT 320: note = Can be replaced by Glu or Asn
  VARIANT 322: note = MOD_RES - Any amino acid or absent
  VARIANT 325: note = Can be replaced by Ser
  VARIANT 326: note = Can be replaced by Ala
  VARIANT 329: note = Can be replaced by Lys or Glu
  VARIANT 331: note = Can be replaced by Phe
  VARIANT 332: note = Can be replaced by Asp
  VARIANT 333: note = MOD_RES - Any amino acid or absent
  VARIANT 335: note = MOD_RES - Any amino acid or absent
  VARIANT 336: note = MOD_RES - Any amino acid or absent
  VARIANT 339: note = MOD_RES - Any amino acid or absent
  VARIANT 340: note = MOD_RES - Any amino acid or absent
  VARIANT 343: note = MOD_RES - Any amino acid or absent
  VARIANT 344: note = MOD_RES - Any amino acid or absent
  VARIANT 345: note = Can be replaced by Val
  VARIANT 346: note = Can be replaced by Lys
  VARIANT 347: note = MOD_RES - Any amino acid or absent
  VARIANT 348: note = MOD_RES - Any amino acid or absent
  VARIANT 349: note = MOD_RES - Any amino acid or absent
  VARIANT 350: note = MOD_RES - Any amino acid or absent
  VARIANT 351: note = MOD_RES - Any amino acid or absent
  VARIANT 352: note = MOD_RES - Any amino acid or absent
  VARIANT 354: note = Can be replaced by Lys or Glu
  VARIANT 356: note = Can be replaced by Ile
  VARIANT 358: note = MOD_RES - Any amino acid or absent
  VARIANT 359: note = Can be replaced by Asn
  VARIANT 360: note = MOD_RES - Any amino acid or absent
  VARIANT 361: note = MOD_RES - Any amino acid or absent
  VARIANT 363: note = MOD_RES - Any amino acid or absent
  VARIANT 372: note = MOD_RES - Any amino acid or absent
  VARIANT 373: note = Can be replaced by Ala
  VARIANT 374: note = MOD_RES - Any amino acid or absent
  VARIANT 376: note = MOD_RES - Any amino acid or absent
  VARIANT 377: note = MOD_RES - Any amino acid or absent
  VARIANT 380: note = MOD_RES - Any amino acid or absent
  VARIANT 381: note = Can be replaced by Phe
  VARIANT 382: note = Can be replaced by Ile or Met
  VARIANT 384: note = MOD_RES - Any amino acid or absent
  VARIANT 385: note = MOD_RES - Any amino acid or absent
  VARIANT 387: note = MOD_RES - Any amino acid or absent
  VARIANT 388: note = Can be replaced by Glu or Asn
  VARIANT 389: note = Can be replaced by Met or Phe
  VARIANT 390: note = Can be replaced by Asp
  VARIANT 392: note = Can be replaced by Thr or Ala
  VARIANT 393: note = MOD_RES - Any amino acid or absent
  VARIANT 394: note = MOD_RES - Any amino acid or absent
  VARIANT 395: note = Can be replaced by Leu
  VARIANT 396: note = Can be replaced by Ile
  VARIANT 397: note = MOD_RES - Any amino acid or absent
  VARIANT 399: note = Can be replaced by Leu
  VARIANT 400: note = Can be replaced by Asn or Asp
  VARIANT 401: note = Can be replaced by Lys
  VARIANT 403: note = Can be replaced by Asn
  VARIANT 404: note = Can be replaced by Leu
  VARIANT 415: note = Can be replaced by Ala
  VARIANT 418: note = Can be replaced by Tyr
  VARIANT 420: note = Can be replaced by Leu
  VARIANT 423: note = MOD_RES - Any amino acid or absent
  VARIANT 425: note = Can be replaced by Leu
  VARIANT 426: note = MOD_RES - Any amino acid or absent
  VARIANT 429: note = Can be replaced by Leu
  VARIANT 430: note = MOD_RES - Any amino acid or absent
  VARIANT 431: note = Can be replaced by Lys or Gln
  VARIANT 433: note = MOD_RES - Any amino acid or absent
  VARIANT 434: note = MOD_RES - Any amino acid or absent
  VARIANT 435: note = Can be replaced by Tyr
  VARIANT 440: note = MOD_RES - Any amino acid or absent
  VARIANT 441: note = MOD_RES - Any amino acid or absent
  VARIANT 443: note = MOD_RES - Any amino acid or absent
  VARIANT 444: note = Can be replaced by Glu
  VARIANT 445: note = Can be replaced by Lys
  VARIANT 447: note = Can be replaced by Lys
  VARIANT 448: note = MOD_RES - Any amino acid or absent
  VARIANT 449: note = Can be replaced by Ile
  VARIANT 450: note = Can be replaced by Val
  VARIANT 457: note = Can be replaced by Phe
  VARIANT 463: note = MOD_RES - Any amino acid or absent
  VARIANT 465: note = Can be replaced by Asn or Gln
  VARIANT 467: note = MOD_RES - Any amino acid or absent
  VARIANT 471: note = MOD_RES - Any amino acid or absent
  VARIANT 472: note = MOD_RES - Any amino acid or absent
  VARIANT 475: note = MOD_RES - Any amino acid or absent
  VARIANT 476: note = MOD_RES - Any amino acid or absent
  VARIANT 477: note = MOD_RES - Any amino acid or absent
  VARIANT 478: note = MOD_RES - Any amino acid or absent
  VARIANT 480: note = MOD_RES - Any amino acid or absent
  VARIANT 484: note = Can be replaced by Ile
  VARIANT 485: note = Can be replaced by Glu
  VARIANT 486: note = Can be replaced by Asp
  VARIANT 487: note = MOD_RES - Any amino acid or absent
  VARIANT 488: note = Can be replaced by Ile
  VARIANT 490: note = MOD_RES - Any amino acid or absent
  VARIANT 491: note = MOD_RES - Any amino acid or absent
  VARIANT 492: note = MOD_RES - Any amino acid or absent
  VARIANT 495: note = MOD_RES - Any amino acid or absent
  REGION 495..502: note = MISC_FEATURE - RuvC-like domain
  VARIANT 496: note = MOD_RES - Any amino acid or absent
  VARIANT 499: note = Can be replaced by Glu
  VARIANT 500: note = Can be replaced by Lys
  VARIANT 503: note = MOD_RES - Any amino acid or absent
  VARIANT 504: note = MOD_RES - Any amino acid or absent
  VARIANT 506: note = MOD_RES - Any amino acid or absent
  VARIANT 507: note = MOD_RES - Any amino acid or absent
  VARIANT 510: note = Can be replaced by Glu or Lys
  VARIANT 511: note = Can be replaced by Glu
  VARIANT 512: note = Can be replaced by Asn
  VARIANT 520: note = MOD_RES - Any amino acid or absent
  VARIANT 522: note = Can be replaced by Gln
  VARIANT 523: note = MOD_RES - Any amino acid or absent
  VARIANT 524: note = Can be replaced by Tyr
  VARIANT 525: note = MOD_RES - Any amino acid or absent
  VARIANT 534: note = Can be replaced by Arg
  VARIANT 535: note = Can be replaced by Phe
  VARIANT 536: note = MOD_RES - Any amino acid or absent
  VARIANT 537: note = MOD_RES - Any amino acid or absent
  VARIANT 538: note = Can be replaced by Asp
  VARIANT 539: note = MOD_RES - Any amino acid or absent
  VARIANT 540: note = MOD_RES - Any amino acid or absent
  VARIANT 541: note = MOD_RES - Any amino acid or absent
  VARIANT 542: note = MOD_RES - Any amino acid or absent
  VARIANT 543: note = MOD_RES - Any amino acid or absent
  VARIANT 544: note = MOD_RES - Any amino acid or absent
  VARIANT 545: note = Can be replaced by Tyr
  VARIANT 546: note = Can be replaced by Leu
  VARIANT 547: note = MOD_RES - Any amino acid or absent
  VARIANT 548: note = MOD_RES - Any amino acid or absent
  VARIANT 549: note = Can be replaced by Glu or Lys or Gln
  VARIANT 550: note = MOD_RES - Any amino acid or absent
  VARIANT 552: note = Can be replaced by Lys or Glu
  VARIANT 553: note = MOD_RES - Any amino acid or absent
  VARIANT 555: note = MOD_RES - Any amino acid or absent
  REGION 555..575: note = MISC_FEATURE - HNH-like domain
  VARIANT 556: note = MOD_RES - Any amino acid or absent
  VARIANT 557: note = MOD_RES - Any amino acid or absent
  VARIANT 558: note = MOD_RES - Any amino acid or absent
  VARIANT 561: note = MOD_RES - Any amino acid or absent
  VARIANT 562: note = MOD_RES - Any amino acid or absent
  VARIANT 566: note = MOD_RES - Any amino acid or absent
  VARIANT 567: note = MOD_RES - Any amino acid or absent
  VARIANT 568: note = MOD_RES - Any amino acid or absent
  VARIANT 569: note = MOD_RES - Any amino acid or absent
  VARIANT 570: note = Can be replaced by Ile
  VARIANT 571: note = MOD_RES - Any amino acid or absent
  VARIANT 572: note = MOD_RES - Any amino acid or absent
  VARIANT 573: note = MOD_RES - Any amino acid or absent
  VARIANT 574: note = MOD_RES - Any amino acid or absent
  VARIANT 575: note = MOD_RES - Any amino acid or absent
  VARIANT 576: note = Can be replaced by His or Arg
  VARIANT 577: note = MOD_RES - Any amino acid or absent
  VARIANT 578: note = Can be replaced by Ile or Met
  VARIANT 579: note = MOD_RES - Any amino acid or absent
  VARIANT 580: note = MOD_RES - Any amino acid or absent
  VARIANT 581: note = MOD_RES - Any amino acid or absent
  VARIANT 582: note = MOD_RES - Any amino acid or absent
  VARIANT 583: note = MOD_RES - Any amino acid or absent
  VARIANT 584: note = MOD_RES - Any amino acid or absent
  VARIANT 585: note = MOD_RES - Any amino acid or absent
  VARIANT 586: note = Can be replaced by Ile
  VARIANT 587: note = MOD_RES - Any amino acid or absent
  VARIANT 589: note = Can be replaced by Val or Ile
  VARIANT 590: note = Can be replaced by Glu
  VARIANT 591: note = MOD_RES - Any amino acid or absent
  VARIANT 592: note = MOD_RES - Any amino acid or absent
  VARIANT 593: note = MOD_RES - Any amino acid or absent
  VARIANT 594: note = MOD_RES - Any amino acid or absent
  VARIANT 595: note = MOD_RES - Any amino acid or absent
  VARIANT 598: note = Can be replaced by Ser
  VARIANT 600: note = MOD_RES - Any amino acid or absent
  VARIANT 601: note = MOD_RES - Any amino acid or absent
  VARIANT 607: note = MOD_RES - Any amino acid or absent
  VARIANT 608: note = Can be replaced by Asn
  VARIANT 609: note = Can be replaced by Val
  VARIANT 610: note = MOD_RES - Any amino acid or absent
  VARIANT 611: note = MOD_RES - Any amino acid or absent
  VARIANT 612: note = MOD_RES - Any amino acid or absent
  VARIANT 613: note = Can be replaced by Gln
  VARIANT 614: note = Can be replaced by Glu
  VARIANT 615: note = Can be replaced by Ile
  VARIANT 618: note = Can be replaced by Asp
  VARIANT 619: note = MOD_RES - Any amino acid or absent
  VARIANT 620: note = MOD_RES - Any amino acid or absent
  VARIANT 622: note = MOD_RES - Any amino acid or absent
  VARIANT 623: note = MOD_RES - Any amino acid or absent
  VARIANT 624: note = Can be replaced by Met
  VARIANT 625: note = Can be replaced by Ile
  VARIANT 627: note = Can be replaced by Glu or Asn
  VARIANT 629: note = Can be replaced by Ile
  VARIANT 630: note = MOD_RES - Any amino acid or absent
  VARIANT 631: note = MOD_RES - Any amino acid or absent
  VARIANT 634: note = Can be replaced by Ile or Val
  VARIANT 638: note = Can be replaced by Lys
  VARIANT 639: note = MOD_RES - Any amino acid or absent
  VARIANT 642: note = MOD_RES - Any amino acid or absent
  VARIANT 643: note = Can be replaced by Glu or Gln
  VARIANT 644: note = Can be replaced by Gln
  VARIANT 646: note = MOD_RES - Any amino acid or absent
  VARIANT 647: note = MOD_RES - Any amino acid or absent
  VARIANT 648: note = Can be replaced by Phe
  VARIANT 649: note = MOD_RES - Any amino acid or absent
  VARIANT 650: note = MOD_RES - Any amino acid or absent
  VARIANT 651: note = Can be replaced by Ile or Val
  VARIANT 652: note = Can be replaced by Phe
  VARIANT 653: note = MOD_RES - Any amino acid or absent
  VARIANT 654: note = MOD_RES - Any amino acid or absent
  VARIANT 655: note = MOD_RES - Any amino acid or absent
  VARIANT 656: note = MOD_RES - Any amino acid or absent
  VARIANT 657: note = Can be replaced by Met or Leu
  VARIANT 659: note = Can be replaced by Gln
  VARIANT 661: note = MOD_RES - Any amino acid or absent
  VARIANT 664: note = Can be replaced by Arg
  VARIANT 670: note = Can be replaced by Lys
  REGION 670..677: note = MISC_FEATURE - RuvC-like domain
  VARIANT 673: note = MOD_RES - Any amino acid or absent
  VARIANT 674: note = Can be replaced by Lys
  VARIANT 676: note = Can be replaced by Leu
  VARIANT 677: note = MOD_RES - Any amino acid or absent
  VARIANT 681: note = Can be replaced by Asp
  VARIANT 682: note = Can be replaced by Glu
  VARIANT 683: note = Can be replaced by Gln or Lys
  VARIANT 685: note = MOD_RES - Any amino acid or absent
  VARIANT 686: note = MOD_RES - Any amino acid or absent
  VARIANT 691: note = Can be replaced by Phe
  VARIANT 693: note = MOD_RES - Any amino acid or absent
  VARIANT 694: note = MOD_RES - Any amino acid or absent
  VARIANT 696: note = MOD_RES - Any amino acid or absent
  VARIANT 697: note = MOD_RES - Any amino acid or absent
  VARIANT 698: note = MOD_RES - Any amino acid or absent
  VARIANT 702: note = Can be replaced by Leu
  VARIANT 707: note = Can be replaced by His
  VARIANT 709: note = MOD_RES - Any amino acid or absent
  VARIANT 710: note = MOD_RES - Any amino acid or absent
  VARIANT 712: note = Can be replaced by Thr
  VARIANT 715: note = MOD_RES - Any amino acid or absent
  VARIANT 716: note = MOD_RES - Any amino acid or absent
  VARIANT 718: note = MOD_RES - Any amino acid or absent
  VARIANT 720: note = MOD_RES - Any amino acid or absent
  VARIANT 722: note = Can be replaced by Ile
  VARIANT 723: note = MOD_RES - Any amino acid or absent
  VARIANT 724: note = MOD_RES - Any amino acid or absent
  VARIANT 725: note = MOD_RES - Any amino acid or absent
  VARIANT 726: note = MOD_RES - Any amino acid or absent
  VARIANT 727: note = MOD_RES - Any amino acid or absent
  VARIANT 728: note = MOD_RES - Any amino acid or absent
  VARIANT 729: note = MOD_RES - Any amino acid or absent
  VARIANT 730: note = MOD_RES - Any amino acid or absent
  VARIANT 731: note = Can be replaced by Ile
  VARIANT 732: note = Can be replaced by His or Lys or Gln
  VARIANT 733: note = MOD_RES - Any amino acid or absent
  VARIANT 734: note = MOD_RES - Any amino acid or absent
  VARIANT 735: note = Can be replaced by Ile
  VARIANT 736: note = MOD_RES - Any amino acid or absent
  VARIANT 737: note = MOD_RES - Any amino acid or absent
  VARIANT 738: note = Can be replaced by Leu
  VARIANT 739: note = MOD_RES - Any amino acid or absent
  VARIANT 751: note = Can be replaced by Thr
  VARIANT 752: note = Can be replaced by Val or Ile
  VARIANT 754: note = Can be replaced by Val
  VARIANT 760: note = MOD_RES - Any amino acid or absent
  VARIANT 761: note = Can be replaced by Val
  VARIANT 764: note = MOD_RES - Any amino acid or absent
  VARIANT 765: note = MOD_RES - Any amino acid or absent
  VARIANT 766: note = MOD_RES - Any amino acid or absent
  VARIANT 768: note = Can be replaced by Gln
  VARIANT 769: note = MOD_RES - Any amino acid or absent
  VARIANT 772: note = Can be replaced by Ile
  VARIANT 780: note = MOD_RES - Any amino acid or absent
  VARIANT 782: note = MOD_RES - Any amino acid or absent
  VARIANT 783: note = Can be replaced by Lys
  VARIANT 785: note = MOD_RES - Any amino acid or absent
  VARIANT 786: note = MOD_RES - Any amino acid or absent
  VARIANT 789: note = Can be replaced by Arg
  VARIANT 790: note = MOD_RES - Any amino acid or absent
  VARIANT 792: note = MOD_RES - Any amino acid or absent
  VARIANT 794: note = MOD_RES - Any amino acid or absent
  VARIANT 795: note = Can be replaced by Ile
  VARIANT 796: note = MOD_RES - Any amino acid or absent
  VARIANT 797: note = MOD_RES - Any amino acid or absent
  VARIANT 798: note = MOD_RES - Any amino acid or absent
  VARIANT 799: note = Can be replaced by Leu
  VARIANT 802: note = Can be replaced by Leu
  VARIANT 805: note = Can be replaced by Lys
  VARIANT 810: note = Can be replaced by Asn
  VARIANT 811: note = MOD_RES - Any amino acid or absent
  VARIANT 812: note = MOD_RES - Any amino acid or absent
  VARIANT 813: note = MOD_RES - Any amino acid or absent
  VARIANT 814: note = MOD_RES - Any amino acid or absent
  VARIANT 815: note = MOD_RES - Any amino acid or absent
  VARIANT 816: note = MOD_RES - Any amino acid or absent
  VARIANT 817: note = MOD_RES - Any amino acid or absent
  VARIANT 818: note = MOD_RES - Any amino acid or absent
  VARIANT 819: note = Can be replaced by Asp
  VARIANT 821: note = MOD_RES - Any amino acid or absent
  VARIANT 822: note = MOD_RES - Any amino acid or absent
  VARIANT 824: note = Can be replaced by Arg
  VARIANT 826: note = Can be replaced by Glu or Asn
  VARIANT 827: note = Can be replaced by Lys
  VARIANT 829: note = Can be replaced by Tyr
  VARIANT 837: note = Can be replaced by Lys
  VARIANT 841: note = MOD_RES - Any amino acid or absent
  VARIANT 842: note = MOD_RES - Any amino acid or absent
  VARIANT 843: note = Can be replaced by Gln or Asp
  VARIANT 844: note = Can be replaced by Asp
  VARIANT 848: note = MOD_RES - Any amino acid or absent
  VARIANT 849: note = MOD_RES - Any amino acid or absent
  VARIANT 852: note = Can be replaced by Asp or Asn
  VARIANT 855: note = Can be replaced by Val
  VARIANT 859: note = Can be replaced by Val
  VARIANT 862: note = Can be replaced by Ser
  VARIANT 864: note = Can be replaced by Leu
  VARIANT 865: note = MOD_RES - Any amino acid or absent
  VARIANT 867: note = Can be replaced by Asp
  VARIANT 872: note = MOD_RES - Any amino acid or absent
  VARIANT 875: note = MOD_RES - Any amino acid or absent
  VARIANT 876: note = MOD_RES - Any amino acid or absent
  VARIANT 878: note = MOD_RES - Any amino acid or absent
  VARIANT 879: note = MOD_RES - Any amino acid or absent
  VARIANT 882: note = MOD_RES - Any amino acid or absent
  VARIANT 884: note = MOD_RES - Any amino acid or absent
  VARIANT 886: note = Can be replaced by Asn
  VARIANT 889: note = MOD_RES - Any amino acid or absent
  VARIANT 890: note = MOD_RES - Any amino acid or absent
  VARIANT 891: note = Can be replaced by Glu
  VARIANT 892: note = Can be replaced by Ile
  VARIANT 894: note = Can be replaced by Lys
  VARIANT 896: note = MOD_RES - Any amino acid or absent
  VARIANT 898: note = MOD_RES - Any amino acid or absent
  VARIANT 899: note = Can be replaced by Phe
  VARIANT 901: note = MOD_RES - Any amino acid or absent
  VARIANT 902: note = Can be replaced by Gln
  VARIANT 904: note = MOD_RES - Any amino acid or absent
  VARIANT 905: note = MOD_RES - Any amino acid or absent
  VARIANT 906: note = MOD_RES - Any amino acid or absent
  VARIANT 907: note = Can be replaced by Asn
  VARIANT 909: note = Can be replaced by Met
  VARIANT 910: note = Can be replaced by Ser
  VARIANT 911: note = Can be replaced by Lys
  VARIANT 916: note = MOD_RES - Any amino acid or absent
  VARIANT 926: note = Can be replaced by Ser
  VARIANT 927: note = MOD_RES - Any amino acid or absent
  VARIANT 928: note = MOD_RES - Any amino acid or absent
  VARIANT 932: note = MOD_RES - Any amino acid or absent
  VARIANT 935: note = Can be replaced by Gln or His
  VARIANT 947: note = Can be replaced by Asn
  VARIANT 950: note = MOD_RES - Any amino acid or absent
  VARIANT 951: note = Can be replaced by Leu
  VARIANT 953: note = MOD_RES - Any amino acid or absent
  VARIANT 954: note = MOD_RES - Any amino acid or absent
  VARIANT 955: note = Can be replaced by Lys
  VARIANT 956: note = Can be replaced by Met
  VARIANT 958: note = MOD_RES - Any amino acid or absent
  VARIANT 959: note = Can be replaced by Lys
  VARIANT 960: note = MOD_RES - Any amino acid or absent
  VARIANT 962: note = Can be replaced by Asp
  VARIANT 963: note = Can be replaced by His
  VARIANT 964: note = MOD_RES - Any amino acid or absent
  VARIANT 965: note = MOD_RES - Any amino acid or absent
  VARIANT 966: note = MOD_RES - Any amino acid or absent
  VARIANT 967: note = Can be replaced by Val or Met
  VARIANT 968: note = Can be replaced by Lys
  VARIANT 969: note = MOD_RES - Any amino acid or absent
  VARIANT 971: note = Can be replaced by Arg
  VARIANT 972: note = Can be replaced by Val
  VARIANT 973: note = Can be replaced by Ile
  VARIANT 978: note = MOD_RES - Any amino acid or absent
  VARIANT 982: note = Can be replaced by Asp or Gln
  VARIANT 986: note = Can be replaced by Asp or Gln
  VARIANT 988: note = Can be replaced by Gln
  VARIANT 989: note = Can be replaced by Phe
  VARIANT 994: note = Can be replaced by Asp
  VARIANT 995: note = Can be replaced by Val
  VARIANT 997: note = Can be replaced by Asn
  VARIANT 998: note = Can be replaced by Phe
  VARIANT 1008: note = MOD_RES - Any amino acid or absent
  VARIANT 1010: note = Can be replaced by Val
  VARIANT 1011: note = MOD_RES - Any amino acid or absent
  VARIANT 1012: note = MOD_RES - Any amino acid or absent
  VARIANT 1013: note = Can be replaced by Thr
  VARIANT 1015: note = Can be replaced by Ile
  VARIANT 1016: note = MOD_RES - Any amino acid or absent
  VARIANT 1017: note = MOD_RES - Any amino acid or absent
  VARIANT 1020: note = Can be replaced by Lys
  VARIANT 1023: note = MOD_RES - Any amino acid or absent
  VARIANT 1031: note = MOD_RES - Any amino acid or absent
  VARIANT 1032: note = MOD_RES - Any amino acid or absent
  VARIANT 1033: note = Can be replaced by Tyr
  VARIANT 1034: note = MOD_RES - Any amino acid or absent
  VARIANT 1035: note = MOD_RES - Any amino acid or absent
  VARIANT 1036: note = MOD_RES - Any amino acid or absent
  VARIANT 1037: note = Can be replaced by Arg
  VARIANT 1038: note = MOD_RES - Any amino acid or absent
  VARIANT 1039: note = MOD_RES - Any amino acid or absent
  VARIANT 1040: note = MOD_RES - Any amino acid or absent
  VARIANT 1041: note = MOD_RES - Any amino acid or absent
  VARIANT 1042: note = MOD_RES - Any amino acid or absent
  VARIANT 1043: note = MOD_RES - Any amino acid or absent
  VARIANT 1044: note = MOD_RES - Any amino acid or absent
  VARIANT 1045: note = MOD_RES - Any amino acid or absent
  VARIANT 1046: note = MOD_RES - Any amino acid or absent
  VARIANT 1047: note = MOD_RES - Any amino acid or absent
  VARIANT 1049: note = MOD_RES - Any amino acid or absent
  VARIANT 1052: note = MOD_RES - Any amino acid or absent
  VARIANT 1054: note = MOD_RES - Any amino acid or absent
  VARIANT 1055: note = MOD_RES - Any amino acid or absent
  VARIANT 1058: note = Can be replaced by Thr
  VARIANT 1062: note = MOD_RES - Any amino acid or absent
  VARIANT 1063: note = Can be replaced by Ile
  VARIANT 1065: note = MOD_RES - Any amino acid or absent
  VARIANT 1066: note = MOD_RES - Any amino acid or absent
  VARIANT 1067: note = MOD_RES - Any amino acid or absent
  VARIANT 1068: note = MOD_RES - Any amino acid or absent
  VARIANT 1069: note = MOD_RES - Any amino acid or absent
  VARIANT 1070: note = MOD_RES - Any amino acid or absent
  VARIANT 1071: note = MOD_RES - Any amino acid or absent
  VARIANT 1072: note = Can be replaced by Asn
  VARIANT 1073: note = MOD_RES - Any amino acid or absent
  VARIANT 1074: note = MOD_RES - Any amino acid or absent
  VARIANT 1075: note = MOD_RES - Any amino acid or absent
  VARIANT 1076: note = MOD_RES - Any amino acid or absent
  VARIANT 1077: note = MOD_RES - Any amino acid or absent
  VARIANT 1078: note = MOD_RES - Any amino acid or absent
  VARIANT 1079: note = MOD_RES - Any amino acid or absent
  VARIANT 1080: note = MOD_RES - Any amino acid or absent
  VARIANT 1081: note = MOD_RES - Any amino acid or absent
  VARIANT 1082: note = MOD_RES - Any amino acid or absent
  VARIANT 1083: note = MOD_RES - Any amino acid or absent
  VARIANT 1084: note = MOD_RES - Any amino acid or absent
  VARIANT 1085: note = MOD_RES - Any amino acid or absent
  VARIANT 1086: note = Can be replaced by Glu
  VARIANT 1087: note = MOD_RES - Any amino acid or absent
  VARIANT 1090: note = MOD_RES - Any amino acid or absent
  VARIANT 1091: note = Can be replaced by Val or Leu
  VARIANT 1093: note = MOD_RES - Any amino acid or absent
  VARIANT 1095: note = MOD_RES - Any amino acid or absent
  VARIANT 1096: note = MOD_RES - Any amino acid or absent
  VARIANT 1097: note = MOD_RES - Any amino acid or absent
  VARIANT 1098: note = Can be replaced by Phe or Leu
  VARIANT 1099: note = MOD_RES - Any amino acid or absent
  VARIANT 1100: note = MOD_RES - Any amino acid or absent
  VARIANT 1101: note = Can be replaced by Val
  VARIANT 1102: note = Can be replaced by Arg
  VARIANT 1103: note = Can be replaced by Arg
  VARIANT 1105: note = Can be replaced by Met
  VARIANT 1107: note = MOD_RES - Any amino acid or absent
  VARIANT 1108: note = MOD_RES - Any amino acid or absent
  VARIANT 1110: note = Can be replaced by Met
  VARIANT 1112: note = Can be replaced by Val
  VARIANT 1116: note = MOD_RES - Any amino acid or absent
  VARIANT 1118: note = MOD_RES - Any amino acid or absent
  VARIANT 1120: note = MOD_RES - Any amino acid or absent
  VARIANT 1121: note = MOD_RES - Any amino acid or absent
  VARIANT 1122: note = MOD_RES - Any amino acid or absent
  VARIANT 1123: note = Can be replaced by Leu
  VARIANT 1124: note = MOD_RES - Any amino acid or absent
  VARIANT 1125: note = Can be replaced by Arg
  VARIANT 1126: note = MOD_RES - Any amino acid or absent
  VARIANT 1127: note = MOD_RES - Any amino acid or absent
  VARIANT 1128: note = MOD_RES - Any amino acid or absent
  VARIANT 1129: note = MOD_RES - Any amino acid or absent
  VARIANT 1130: note = MOD_RES - Any amino acid or absent
  VARIANT 1131: note = MOD_RES - Any amino acid or absent
  VARIANT 1132: note = MOD_RES - Any amino acid or absent
  VARIANT 1133: note = MOD_RES - Any amino acid or absent
  VARIANT 1134: note = MOD_RES - Any amino acid or absent
  VARIANT 1135: note = MOD_RES - Any amino acid or absent
  VARIANT 1136: note = MOD_RES - Any amino acid or absent
  VARIANT 1137: note = Can be replaced by Thr
  VARIANT 1138: note = MOD_RES - Any amino acid or absent
  VARIANT 1139: note = MOD_RES - Any amino acid or absent
  VARIANT 1142: note = MOD_RES - Any amino acid or absent
  VARIANT 1145: note = MOD_RES - Any amino acid or absent
  VARIANT 1146: note = Can be replaced by Glu
  VARIANT 1147: note = MOD_RES - Any amino acid or absent
  VARIANT 1149: note = Can be replaced by Val
  VARIANT 1150: note = MOD_RES - Any amino acid or absent
  VARIANT 1151: note = MOD_RES - Any amino acid or absent
  VARIANT 1153: note = MOD_RES - Any amino acid or absent
  VARIANT 1154: note = MOD_RES - Any amino acid or absent
  VARIANT 1155: note = MOD_RES - Any amino acid or absent
  VARIANT 1156: note = MOD_RES - Any amino acid or absent
  VARIANT 1157: note = MOD_RES - Any amino acid or absent
  VARIANT 1158: note = MOD_RES - Any amino acid or absent
  VARIANT 1160: note = MOD_RES - Any amino acid or absent
  VARIANT 1161: note = MOD_RES - Any amino acid or absent
  VARIANT 1166: note = MOD_RES - Any amino acid or absent
  VARIANT 1167: note = MOD_RES - Any amino acid or absent
  VARIANT 1168: note = MOD_RES - Any amino acid or absent
  VARIANT 1169: note = MOD_RES - Any amino acid or absent
  VARIANT 1170: note = MOD_RES - Any amino acid or absent
  VARIANT 1171: note = MOD_RES - Any amino acid or absent
  VARIANT 1172: note = Can be replaced by Ser
  VARIANT 1173: note = Can be replaced by Phe
  VARIANT 1174: note = Can be replaced by Thr or Ala
  VARIANT 1175: note = Can be replaced by Val
  VARIANT 1176: note = Can be replaced by Val
  VARIANT 1177: note = Can be replaced by Ile
  VARIANT 1178: note = MOD_RES - Any amino acid or absent
  VARIANT 1179: note = MOD_RES - Any amino acid or absent
  VARIANT 1180: note = MOD_RES - Any amino acid or absent
  VARIANT 1181: note = MOD_RES - Any amino acid or absent
  VARIANT 1182: note = MOD_RES - Any amino acid or absent
  VARIANT 1185: note = MOD_RES - Any amino acid or absent
  VARIANT 1186: note = MOD_RES - Any amino acid or absent
  VARIANT 1187: note = MOD_RES - Any amino acid or absent
  VARIANT 1189: note = Can be replaced by Ile
  VARIANT 1190: note = MOD_RES - Any amino acid or absent
  VARIANT 1191: note = MOD_RES - Any amino acid or absent
  VARIANT 1192: note = MOD_RES - Any amino acid or absent
  VARIANT 1193: note = MOD_RES - Any amino acid or absent
  VARIANT 1194: note = MOD_RES - Any amino acid or absent
  VARIANT 1195: note = Can be replaced by Phe or Ile
  VARIANT 1196: note = MOD_RES - Any amino acid or absent
  VARIANT 1197: note = MOD_RES - Any amino acid or absent
  VARIANT 1198: note = Can be replaced by Ile
  VARIANT 1199: note = Can be replaced by Ser
  VARIANT 1201: note = Can be replaced by Leu
  VARIANT 1202: note = Can be replaced by Asp
  VARIANT 1203: note = Can be replaced by Arg
  VARIANT 1204: note = MOD_RES - Any amino acid or absent
  VARIANT 1205: note = MOD_RES - Any amino acid or absent
  VARIANT 1206: note = Can be replaced by Tyr
  VARIANT 1207: note = MOD_RES - Any amino acid or absent
  VARIANT 1208: note = Can be replaced by Lys
  VARIANT 1209: note = Can be replaced by Asn
  VARIANT 1210: note = MOD_RES - Any amino acid or absent
  VARIANT 1211: note = MOD_RES - Any amino acid or absent
  VARIANT 1212: note = MOD_RES - Any amino acid or absent
  VARIANT 1215: note = MOD_RES - Any amino acid or absent
  VARIANT 1216: note = MOD_RES - Any amino acid or absent
  VARIANT 1217: note = Can be replaced by Gln
  VARIANT 1220: note = Can be replaced by Lys
  VARIANT 1221: note = Can be replaced by Glu or Asp or Gln
  VARIANT 1222: note = MOD_RES - Any amino acid or absent
  VARIANT 1223: note = MOD_RES - Any amino acid or absent
  VARIANT 1224: note = MOD_RES - Any amino acid or absent
  VARIANT 1225: note = MOD_RES - Any amino acid or absent
  VARIANT 1226: note = MOD_RES - Any amino acid or absent
  VARIANT 1227: note = Can be replaced by Leu
  VARIANT 1228: note = MOD_RES - Any amino acid or absent
  VARIANT 1229: note = Can be replaced by Glu
  VARIANT 1234: note = Can be replaced by Thr
  VARIANT 1236: note = Can be replaced by Tyr
  VARIANT 1237: note = Can be replaced by Glu
  VARIANT 1238: note = MOD_RES - Any amino acid or absent
  VARIANT 1239: note = MOD_RES - Any amino acid or absent
  VARIANT 1240: note = Can be replaced by Asp or Glu
  VARIANT 1242: note = MOD_RES - Any amino acid or absent
  VARIANT 1243: note = Can be replaced by Arg
  VARIANT 1245: note = Can be replaced by Met
  VARIANT 1249: note = MOD_RES - Any amino acid or absent
  VARIANT 1250: note = MOD_RES - Any amino acid or absent
  VARIANT 1251: note = MOD_RES - Any amino acid or absent
  VARIANT 1252: note = MOD_RES - Any amino acid or absent
  VARIANT 1253: note = MOD_RES - Any amino acid or absent
  VARIANT 1254: note = MOD_RES - Any amino acid or absent
  VARIANT 1255: note = MOD_RES - Any amino acid or absent
  VARIANT 1256: note = MOD_RES - Any amino acid or absent
  VARIANT 1257: note = MOD_RES - Any amino acid or absent
  VARIANT 1259: note = MOD_RES - Any amino acid or absent
  VARIANT 1260: note = Can be replaced by His
  VARIANT 1264: note = Can be replaced by Gln
  VARIANT 1265: note = MOD_RES - Any amino acid or absent
  VARIANT 1266: note = MOD_RES - Any amino acid or absent
  VARIANT 1268: note = MOD_RES - Any amino acid or absent
  VARIANT 1269: note = MOD_RES - Any amino acid or absent
  VARIANT 1270: note = Can be replaced by Lys
  VARIANT 1271: note = MOD_RES - Any amino acid or absent
  VARIANT 1272: note = MOD_RES - Any amino acid or absent
  VARIANT 1273: note = MOD_RES - Any amino acid or absent
  VARIANT 1274: note = Can be replaced by Phe
  VARIANT 1276: note = Can be replaced by His
  VARIANT 1277: note = MOD_RES - Any amino acid or absent
  VARIANT 1279: note = MOD_RES - Any amino acid or absent
  VARIANT 1280: note = MOD_RES - Any amino acid or absent
  VARIANT 1281: note = MOD_RES - Any amino acid or absent
  VARIANT 1282: note = MOD_RES - Any amino acid or absent
  VARIANT 1283: note = MOD_RES - Any amino acid or absent
  VARIANT 1284: note = MOD_RES - Any amino acid or absent
  VARIANT 1285: note = MOD_RES - Any amino acid or absent
  VARIANT 1286: note = MOD_RES - Any amino acid or absent
  VARIANT 1287: note = MOD_RES - Any amino acid or absent
  VARIANT 1288: note = MOD_RES - Any amino acid or absent
  VARIANT 1289: note = MOD_RES - Any amino acid or absent
  VARIANT 1290: note = MOD_RES - Any amino acid or absent
  VARIANT 1291: note = MOD_RES - Any amino acid or absent
  VARIANT 1292: note = Can be replaced by Asn or Asp
  VARIANT 1293: note = MOD_RES - Any amino acid or absent
  VARIANT 1294: note = Can be replaced by Asn
  VARIANT 1295: note = MOD_RES - Any amino acid or absent
  VARIANT 1296: note = MOD_RES - Any amino acid or absent
  VARIANT 1297: note = MOD_RES - Any amino acid or absent
  VARIANT 1298: note = Can be replaced by Phe
  VARIANT 1299: note = Can be replaced by Ile
  VARIANT 1300: note = Can be replaced by Glu
  VARIANT 1301: note = MOD_RES - Any amino acid or absent
  VARIANT 1302: note = Can be replaced by Asn
  VARIANT 1303: note = Can be replaced by Arg
  VARIANT 1304: note = MOD_RES - Any amino acid or absent
  VARIANT 1305: note = MOD_RES - Any amino acid or absent
  VARIANT 1306: note = Can be replaced by Leu
  VARIANT 1307: note = MOD_RES - Any amino acid or absent
  VARIANT 1309: note = Can be replaced by Ile
  VARIANT 1310: note = Can be replaced by Ile or Phe
  VARIANT 1311: note = MOD_RES - Any amino acid or absent
  VARIANT 1312: note = MOD_RES - Any amino acid or absent
  VARIANT 1313: note = Can be replaced by Ile
  VARIANT 1314: note = MOD_RES - Any amino acid or absent
  VARIANT 1315: note = Can be replaced by Glu
  VARIANT 1317: note = MOD_RES - Any amino acid or absent
  VARIANT 1318: note = Can be replaced by Glu
  VARIANT 1319: note = MOD_RES - Any amino acid or absent
  VARIANT 1320: note = MOD_RES - Any amino acid or absent
  VARIANT 1321: note = MOD_RES - Any amino acid or absent
  VARIANT 1322: note = MOD_RES - Any amino acid or absent
  VARIANT 1324: note = MOD_RES - Any amino acid or absent
  VARIANT 1325: note = MOD_RES - Any amino acid or absent
  VARIANT 1327: note = MOD_RES - Any amino acid or absent
  VARIANT 1328: note = MOD_RES - Any amino acid or absent
  VARIANT 1329: note = MOD_RES - Any amino acid or absent
  VARIANT 1330: note = Can be replaced by Val or Leu
  VARIANT 1331: note = MOD_RES - Any amino acid or absent
  VARIANT 1332: note = MOD_RES - Any amino acid or absent
  VARIANT 1333: note = MOD_RES - Any amino acid or absent
  VARIANT 1334: note = Can be replaced by Phe
  VARIANT 1335: note = MOD_RES - Any amino acid or absent
  VARIANT 1336: note = MOD_RES - Any amino acid or absent
  VARIANT 1337: note = MOD_RES - Any amino acid or absent
  VARIANT 1338: note = MOD_RES - Any amino acid or absent
  VARIANT 1339: note = MOD_RES - Any amino acid or absent
  VARIANT 1340: note = MOD_RES - Any amino acid or absent
  VARIANT 1341: note = MOD_RES - Any amino acid or absent
  VARIANT 1342: note = Can be replaced by Ile
  VARIANT 1343: note = MOD_RES - Any amino acid or absent
  VARIANT 1344: note = MOD_RES - Any amino acid or absent
  VARIANT 1345: note = MOD_RES - Any amino acid or absent
  VARIANT 1346: note = MOD_RES - Any amino acid or absent
  VARIANT 1347: note = MOD_RES - Any amino acid or absent
  VARIANT 1348: note = MOD_RES - Any amino acid or absent
  VARIANT 1349: note = Can be replaced by Ile
  VARIANT 1350: note = Can be replaced by Val
  VARIANT 1351: note = MOD_RES - Any amino acid or absent
  VARIANT 1352: note = MOD_RES - Any amino acid or absent
  VARIANT 1353: note = MOD_RES - Any amino acid or absent
  VARIANT 1354: note = MOD_RES - Any amino acid or absent
  VARIANT 1355: note = MOD_RES - Any amino acid or absent
  VARIANT 1356: note = MOD_RES - Any amino acid or absent
  VARIANT 1357: note = MOD_RES - Any amino acid or absent
  VARIANT 1358: note = MOD_RES - Any amino acid or absent
  VARIANT 1359: note = MOD_RES - Any amino acid or absent
  VARIANT 1361: note = Can be replaced by Phe or Met
  VARIANT 1362: note = MOD_RES - Any amino acid or absent
  VARIANT 1363: note = Can be replaced by Leu
  VARIANT 1364: note = MOD_RES - Any amino acid or absent
  VARIANT 1365: note = MOD_RES - Any amino acid or absent
  VARIANT 1366: note = MOD_RES - Any amino acid or absent
  VARIANT 1368: note = Can be replaced by Ser
  VARIANT 1369: note = MOD_RES - Any amino acid or absent
  VARIANT 1371: note = MOD_RES - Any amino acid or absent
  VARIANT 1373: note = Can be replaced by Glu
  VARIANT 1374: note = Can be replaced by Tyr
  VARIANT 1375: note = Can be replaced by Leu
  VARIANT 1376: note = MOD_RES - Any amino acid or absent
  VARIANT 1377: note = MOD_RES - Any amino acid or absent
  VARIANT 1378: note = MOD_RES - Any amino acid or absent
  VARIANT 1380: note = MOD_RES - Any amino acid or absent
  VARIANT 1382: note = MOD_RES - Any amino acid or absent
  VARIANT 1384: note = MOD_RES - Any amino acid or absent
  VARIANT 1386: note = MOD_RES - Any amino acid or absent
  VARIANT 1387: note = MOD_RES - Any amino acid or absent
  VARIANT 1388: note = MOD_RES - Any amino acid or absent
  VARIANT 1389: note = MOD_RES - Any amino acid or absent
  VARIANT 1390: note = MOD_RES - Any amino acid or absent
  VARIANT 1391: note = Can be replaced by Val or Leu
  VARIANT 1392: note = MOD_RES - Any amino acid or absent
  VARIANT 1393: note = Can be replaced by Asp
  VARIANT 1394: note = Can be replaced by Ser
  VARIANT 1396: note = Can be replaced by Ile
  VARIANT 1398: note = Can be replaced by Tyr
  VARIANT 1401: note = Can be replaced by Val
  VARIANT 1407: note = Can be replaced by Ser
  VARIANT 1409: note = MOD_RES - Any amino acid or absent
  VARIANT 1410: note = MOD_RES - Any amino acid or absent
  VARIANT 1412: note = MOD_RES - Any amino acid or absent
  VARIANT 1413: note = MOD_RES - Any amino acid or absent
  VARIANT 1414: note = MOD_RES - Any amino acid or absent
  VARIANT 1415: note = MOD_RES - Any amino acid or absent
  VARIANT 1416: note = MOD_RES - Any amino acid or absent
  VARIANT 1417: note = MOD_RES - Any amino acid or absent
  REGION 1..1417: note = MISC_FEATURE - Variant residues given in the sequence have no preference with respect to those in the annotations for variant positions
  REGION 1..1417: note = Description of Artificial Sequence: Synthetic polypeptide
  REGION 1..1417: note = Synthetic Cas9 consensus sequence derived from Sm, Sp, St, and Li Cas9 sequences
MXKXYSIGLDIGTNSVGWAVXTDDYXVXXKKMKVXGNTXKXXIEKNLXGXXLFDXGXTAXXXRLXRTARRRXXRRRNRIXYLQXIFSXEMXXXDXXFFXRLXDSFXVXEDKRXXRHPIFXXLXEEXXYHXNFPTIYHLRQXLXDXXXKXDLRLVYLALAHIIKFRGNFLIEGXFDXXNXXVQXXFXEFLXXYDXXFXXXXXXXXXXXXXXXXQXXXILXXKISKXXKXDXVLXXXPXEKXXGXFXEFLXLXVGXXXDFKXXFELXEXAXLXXSKDTYEEELEXLLXXIGDNYAELFLXAKKXXXSILLSXILXVXXXXTKAXLSASMIQRYNXHXXDLXXLKXXIRXXXXXXYNEVFXDXXKXGYAGYIDGXTXQXXFYXYLKXXLXKIEGSXXFLXKIEREDFLRKQRTFDNGSIPHQIHLXEMXAIIXRQXXFYPFLXXNXDRIEXLLTFRIPYYVGPLAXGKSXFAWXXRKXXXXIXPWNFDEXVDXXXSAXXFINRMTXXDXXLPNQKVLPKHSLXYEXFXVYNELTKVKYXXEXXXXXXFFXXNXKQXIXXXXFKXXRKVXXXXLXXXXXKXFXXXXXXXLXGLDXXXXXFNASXXTYHDLXKIXXXKDFLDNXXNXXILEDIVXXLTLFEDRXMIXKRLXXYXXLLXXXXVKKLXRRHYTGWGRLSXELIXGIRNKESXXTILDYLXXDXXXNRNFMQLINDXXLSFKXXIXKXQVXXXXXXXXLNXXVXXIXGSPAIKKGILQSLKIVDELVXIMGXXXPEXIVVEMARENQXTXQGXXNSQXRXKXLXXXIKEFGSQILKEHXXXXXXXXENXXLQNDRLFLYYLQNGRDMYXXEELDIXXLSQYDIDHIIPQAFIXDNSIDNXVLXXSXXNRXKXDDVPXXDVVRKXKXYWXKLXXXKLITQRKFDXLTKAERGGLTXXDKAXFIKRQLVETRQITKHVAXILXXRFNXEXDENXXXIRXVKIVTLKSXLVSNFRKEFELLYKVEINDYHHAHDAYLNXVIXXALLXXYPQLEXEFVYGDYXXFXXXKXXXXXXXXXXKXATXKXXFYSNIMXFFXXXXXXXDXXXXXXXXXXXXXKXGEXIWXKXXXIXXIKKVLMXXQVNIVKKXEXQXXXFXKXXXXXXXXXXXSXXPKXNSXKXLIXXKXXXXXXDXXKYGGXXXXXXAYSILVXXXXXKGXXXKLXXXXXLXXVTIMEKXXFXRDXXXFLXXKGYRNXXXXXIXKLPKYSLFKXXNGXKRLLASXXXXXXXXXEXQKGNEXXLXXHXXXLLYXAXXXXXXXXXXXXXEXKXXXYVDXHKXXFXELLXXVXNFXKXXXXAXXNXXXIXXXYXXXXXXXLXXXXXXFIXXXXXXXXXLLXFXXXGAXAXFKFFXXXIXRXRXYXXXXXIXNATLIHQSITGLYETRXXLXXXXXX

SEQ ID NO: 15-37 - skipped (23 entries)

SEQ ID NO: 38 - RNA, 102 nt, synthetic construct
  misc_difference 1..20: note = modified_base - n is a, c, g, or u
  misc_feature 1..20: note = Targeting domain
  misc_feature 1..102: note = Description of Artificial Sequence: Synthetic polynucleotide
  misc_feature 1..102: note = Unimolecular gRNA derived from S. aureus
  source 1..102: mol_type = other RNA
nnnnnnnnnnnnnnnnnnnngttttagtactctggaaacagaatctactaaaacaaggcaaaatgccgtgtttatctcgtcaacttgttggcgagatttttt

SEQ ID NO: 39 - RNA, 42 nt, synthetic construct
  misc_difference 1..20: note = modified_base - a, c, u, g, unknown or other
  misc_feature 1..20: note = Targeting domain
  misc_feature 21..42: note = First complementarity domain
  misc_feature 1..42: note = Description of Artificial Sequence: Synthetic oligonucleotide
  misc_feature 1..42: note = Modular gRNA derived from S. pyogenes
  source 1..42: mol_type = other RNA
nnnnnnnnnnnnnnnnnnnngttttagagctatgctgttttg

SEQ ID NO: 40 - RNA, 85 nt, synthetic construct
  misc_feature 1..9: note = 5' extension domain
  misc_feature 10..33: note = Second complementarity domain
  misc_feature 34..45: note = Proximal domain
  misc_feature 46..85: note = Tail domain
  misc_feature 1..85: note = Description of Artificial Sequence: Synthetic oligonucleotide
  misc_feature 1..85: note = Modular gRNA derived from S. pyogenes
  source 1..85: mol_type = other RNA
ggaaccattcaaaacagcatagcaagttaaaataaggctagtccgttatcaacttgaaaaagtggcaccgagtcggtgctttttt

SEQ ID NO: 41 - RNA, 62 nt, synthetic construct
  misc_difference 1..20: note = modified_base - a, c, u, g, unknown or other
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  misc_feature 1..62: note = Description of Artificial Sequence: Synthetic oligonucleotide
  misc_feature 1..62: note = Unimolecular gRNA derived from S. pyogenes
  source 1..62: mol_type = other RNA
nnnnnnnnnnnnnnnnnnnngttttagagctagaaatagcaagttaaaataaggctagtccg

SEQ ID NO: 42 - RNA, 102 nt, synthetic construct
  misc_difference 1..20: note = modified_base - a, c, u, g, unknown or other
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  misc_feature 63..102: note = Tail domain
  misc_feature 1..102: note = Description of Artificial Sequence: Synthetic polynucleotide
  misc_feature 1..102: note = Unimolecular gRNA derived from S. pyogenes
  source 1..102: mol_type = other RNA
nnnnnnnnnnnnnnnnnnnngttttagagctagaaatagcaagttaaaataaggctagtccgttatcaacttgaaaaagtggcaccgagtcggtgctttttt

SEQ ID NO: 43 - RNA, 75 nt, synthetic construct
  misc_feature 1..20: note = Targeting domain
  misc_difference 1..20: note = modified_base - a, c, u, g, unknown or other
  misc_feature 21..36: note = First complementarity domain
  misc_feature 37..40: note = Linking domain
  misc_feature 41..58: note = Second complementarity domain
  misc_feature 59..70: note = Proximal domain
  misc_feature 71..75: note = Tail domain
  misc_feature 1..75: note = Description of Artificial Sequence: Synthetic oligonucleotide
  misc_feature 1..75: note = Unimolecular gRNA derived from S. pyogenes
  source 1..75: mol_type = other RNA
nnnnnnnnnnnnnnnnnnnngttttagagctatgctgaaaagcatagcaagttaaaataaggctagtccgttatc

SEQ ID NO: 44 - RNA, 87 nt, synthetic construct
  misc_difference 1..20: note = modified_base - a, c, u, g, unknown or other
  misc_feature 1..20: note = Targeting domain
  misc_feature 21..32: note = First complementarity domain
  misc_feature 43..46: note = Linking domain
  misc_feature 57..70: note = Second complementarity domain
  misc_feature 71..82: note = Proximal domain
  misc_feature 83..87: note = Tail domain
  misc_feature 1..87: note = Description of Artificial Sequence: Synthetic oligonucleotide
  misc_feature 1..87: note = Unimolecular gRNA derived from S. pyogenes
  source 1..87: mol_type = other RNA
nnnnnnnnnnnnnnnnnnnngttttagagctatgctgttttggaaacaaaacagcatagcaagttaaaataaggctagtccgttatc

SEQ ID NO: 45 - RNA, 42 nt, synthetic construct
  misc_difference 1..20: note = modified_base - a, c, u, g, unknown or other
  misc_feature 1..20: note = Targeting domain
  misc_feature 21..42: note = First complementarity domain
  misc_feature 1..42: note = Description of Artificial Sequence: Synthetic oligonucleotide
  misc_feature 1..42: note = Modular gRNA derived from S. thermophilus
  source 1..42: mol_type = other RNA
nnnnnnnnnnnnnnnnnnnngttttagagctgtgttgtttcg

SEQ ID NO: 46 - RNA, 78 nt, synthetic construct
  misc_feature 1..3: note = 5' extension domain
  misc_feature 4..27: note = Second complementarity domain
  misc_feature 28..40: note = Proximal domain
  misc_feature 41..78: note = Tail domain
  misc_feature 1..78: note = Description of Artificial Sequence: Synthetic oligonucleotide
  misc_feature 1..78: note = Modular gRNA derived from S. thermophilus
  source 1..78: mol_type = other RNA
gggcgaaacaacacagcgagttaaaataaggcttagtccgtactcaacttgaaaaggtggcaccgattcggtgttttt

SEQ ID NO: 47 - RNA, 85 nt, synthetic construct
  misc_feature 1..85: note = Description of Artificial Sequence: Synthetic oligonucleotide
  misc_feature 1..85: note = Modular gRNA derived from S. pyogenes
  source 1..85: mol_type = other RNA
gaaccattcaaaacagcatagcaagttaaaataaggctagtccgttatcaacttgaaaaagtggcaccgagtcggtgcttttttt

SEQ ID NO: 48-51 - skipped (4 entries)

SEQ ID NO: 52 - protein, 12 aa, Peptoniphilus sp.
  source 1..12: note = Peptoniphilus duerdenii
DIGTASVGWAVT

SEQ ID NO: 53 - protein, 12 aa, Treponema denticola
DVGTGSVGWAVT

SEQ ID NO: 54 - protein, 12 aa, Streptococcus mutans
DIGTNSVGWAVV

SEQ ID NO: 55 - protein, 12 aa, Streptococcus pyogenes
DIGTNSVGWAVI

SEQ ID NO: 56 - protein, 12 aa, Listeria innocua
DIGTNSVGWAVL

SEQ ID NO: 57 - protein, 12 aa, Flavobacterium branchiophilum
  source 1..12: note = Flavobacterium branchiophilum FL-15
DLGTNSIGWAVV

SEQ ID NO: 58 - protein, 11 aa, synthetic construct
  REGION 1..11: note = Description of Artificial Sequence: Synthetic peptide
  REGION 1..11: note = N-terminal RuvC-like domain
DLGTNSIGWAI

SEQ ID NO: 59 - protein, 12 aa, Bacteroides fragilis
  source 1..12: note = Bacteroides fragilis, NCTC 9343
DLGTNSIGWALV

SEQ ID NO: 60 - protein, 12 aa, Fusobacterium nucleatum
DIGTNSVGWCVT

SEQ ID NO: 61 - protein, 12 aa, Acidaminococcus sp.
  source 1..12: note = Acidaminococcus sp. D21
DIGTNSVGYAVT

SEQ ID NO: 62 - protein, 12 aa, Coprococcus catus
  source 1..12: note = Coprococcus catus GD-7
DMGTGSLGWAVT

SEQ ID NO: 63 - protein, 12 aa, Oenococcus kitaharae
  source 1..12: note = Oenococcus kitaharae DSM 17330
DIGTSSVGWAAI

SEQ ID NO: 64 - protein, 12 aa, Catenibacterium mitsuokai
  source 1..12: note = Catenibacterium mitsuokai DSM 15897
DLGTGSVGWAVV

SEQ ID NO: 65 - protein, 12 aa, Mycoplasma gallisepticum
  source 1..12: note = Mycoplasma gallisepticum str. F
DLGVGSVGWAIV

SEQ ID NO: 66 - protein, 12 aa, Mycoplasma ovipneumoniae
  source 1..12: note = Mycoplasma ovipneumoniae SC01
DLGIASIGWAII

SEQ ID NO: 67 - protein, 12 aa, Mycoplasma canis
  source 1..12: note = Mycoplasma canis PG 14
DLGIASVGWAIV

SEQ ID NO: 68 - protein, 12 aa, Mycoplasma synoviae
  source 1..12: note = Mycoplasma synoviae 53
DLGVASVGWSIV

SEQ ID NO: 69 - protein, 12 aa, Eubacterium rectale
DIGIASVGWAIL

SEQ ID NO: 70 - protein, 12 aa, Enterococcus faecalis
  source 1..12: note = Enterococcus faecalis TX0012
DLGISSVGWSVI

SEQ ID NO: 71 - protein, 12 aa, Ilyobacter polytropus
  source 1..12: note = Ilyobacter polytropus DSM 2926
DIGIASVGWSVI

SEQ ID NO: 72 - protein, 12 aa, Ruminococcus albus
  source 1..12: note = Ruminococcus albus 8
DVGIGSIGWAVI

SEQ ID NO: 73 - protein, 12 aa, Elusimicrobium minutum
  source 1..12: note = Elusimicrobium minutum Pei191
DLGVGSIGFAIV

SEQ ID NO: 74 - protein, 12 aa, Akkermansia muciniphila
DIGYASIGWAVI

SEQ ID NO: 75 - protein, 12 aa, synthetic construct
  REGION 1..12: note = Description of Artificial Sequence: Synthetic peptide
  REGION 1..12: note = N-terminal RuvC-like domain
DTGTNSLGWAIV

SEQ ID NO: 76 - protein, 12 aa, unidentified
  source 1..12: note = Candidatus Puniceispirillum marinum
DLGTNSIGWCLL

SEQ ID NO: 77 - protein, 12 aa, Rhodospirillum rubrum
DIGTDSLGWAVF

SEQ ID NO: 78 - protein, 12 aa, Lactobacillus rhamnosus
  source 1..12: note = Lactobacillus rhamnosus GG
DIGSNSIGFAVV

SEQ ID NO: 79 - protein, 12 aa, unidentified
  source 1..12: note = Sphaerochaeta globus str. Buddy
DLGVGSIGVAVA

SEQ ID NO: 80 - protein, 12 aa, synthetic construct
  REGION 1..12: note = Description of Artificial Sequence: Synthetic peptide
  REGION 1..12: note = N-terminal RuvC-like domain
DLGIASCGWGVV

SEQ ID NO: 81 - protein, 12 aa, Mycoplasma mobile
  source 1..12: note = Mycoplasma mobile 163K
DLGIASVGWCLT

SEQ ID NO: 82 - protein, 12 aa, Streptococcus thermophilus
  source 1..12: note = Streptococcus thermophilus LMD-9
DIGIGSVGVGIL

SEQ ID NO: 83 - protein, 12 aa, Staphylococcus lugdunensis
  source 1..12: note = Staphylococcus lugdunensis M23590
DIGITSVGYGLI

SEQ ID NO: 84 - protein, 12 aa, Eubacterium dolichum
  source 1..12: note = Eubacterium dolichum DSM 3991
DIGITSVGFGII

SEQ ID NO: 85 - protein, 12 aa, Lactobacillus coryniformis
  source 1..12: note = Lactobacillus coryniformis KCTC 3535
DVGITSTGYAVL

SEQ ID NO: 86 - protein, 12 aa, Nitratifractor salsuginis
  source 1..12: note = Nitratifractor salsuginis DSM 16511
DLGITSFGYAIL

SEQ ID NO: 87 - protein, 12 aa, Bifidobacterium bifidum
  source 1..12: note = Bifidobacterium bifidum S17
DIGNASVGWSAF

SEQ ID NO: 88 - protein, 12 aa, synthetic construct
  REGION 1..12: note = Description of Artificial Sequence: Synthetic peptide
  REGION 1..12: note = N-terminal RuvC-like domain
DVGTNSCGWVAM

SEQ ID NO: 89 - protein, 12 aa, Acidothermus cellulolyticus
  source 1..12: note = Acidothermus cellulolyticus 11B
DVGERSIGLAAV

SEQ ID NO: 90 - protein, 12 aa, Bifidobacterium longum
  source 1..12: note = Bifidobacterium longum DJO10A
DVGLNSVGLAAV

SEQ ID NO: 91 - protein, 12 aa, synthetic construct
  REGION 1..12: note = Description of Artificial Sequence: Synthetic peptide
  REGION 1..12: note = N-terminal RuvC-like domain
DVGLMSVGLAAI

SEQ ID NO: 92 - protein, 12 aa, synthetic construct
  REGION 1..12: note = Description of Artificial Sequence: Synthetic peptide
  REGION 1..12: note = N-terminal RuvC-like domain
DVGTFSVGLAAI

SEQ ID NO: 93 - protein, 12 aa, Staphylococcus pseudintermedius
  source 1..12: note = Staphylococcus pseudintermedius ED99
DIGTGSVGYACM

SEQ ID NO: 94 - protein, 12 aa, synthetic construct
  REGION 1..12: note = Description of Artificial Sequence: Synthetic peptide
  REGION 1..12: note = N-terminal RuvC-like domain
DLGTTSIGFAHI

SEQ ID NO: 95 - protein, 12 aa, synthetic construct
  REGION 1..12: note = Description of Artificial Sequence: Synthetic peptide
  REGION 1..12: note = N-terminal RuvC-like domain
DLGTNSIGSSVR

SEQ ID NO: 96 - protein, 12 aa, synthetic construct
  REGION 1..12: note = Description of Artificial Sequence: Synthetic peptide
  REGION 1..12: note = N-terminal RuvC-like domain
DIGTNSIGWAVI

SEQ ID NO: 97 - protein, 12 aa, Pasteurella multocida
  source 1..12: note = Pasteurella multocida str. Pm70
DLGIASVGWAVV

SEQ ID NO: 98 - protein, 12 aa, synthetic construct
  REGION 1..12: note = Description of Artificial Sequence: Synthetic peptide
  REGION 1..12: note = N-terminal RuvC-like domain
DIGIASVGWAVL

SEQ ID NO: 99 - protein, 12 aa, Helicobacter mustelae
  source 1..12: note = Helicobacter mustelae 12198
DIGIASIGWAVI

SEQ ID NO: 100 - protein, 12 aa, synthetic construct
  REGION 1..12: note = Description of Artificial Sequence: Synthetic peptide
  REGION 1..12: note = N-terminal RuvC-like domain
DIGIASVGWAII

SEQ ID NO: 101 - protein, 12 aa, Clostridium cellulolyticum
  source 1..12: note = Clostridium cellulolyticum H10
DVGIASVGWAVI

SEQ ID NO: 102 - protein, 11 aa, synthetic construct
  REGION 1..11: note = Description of Artificial Sequence: Synthetic peptide
  REGION 1..11: note = N-terminal RuvC-like domain
DIGIASVGWAL

SEQ ID NO: 103 - protein, 12 aa, Neisseria meningitidis
DIGIASVGWAMV

SEQ ID NO: 104 - protein, 12 aa, synthetic construct
  REGION 1..12: note = Description of Artificial Sequence: Synthetic peptide
  REGION 1..12: note = N-terminal RuvC-like domain
DIGITSVGWAVI

SEQ ID NO: 105 - protein, 12 aa, Wolinella succinogenes
  source 1..12: note = Wolinella succinogenes DSM 1740
DLGISSLGWAIV

SEQ ID NO: 106 - protein, 12 aa, Azospirillum sp.
  source 1..12: note = Azospirillum sp. B510
DLGTNSIGWGLL

SEQ ID NO: 107 - protein, 12 aa, synthetic construct
  REGION 1..12: note = Description of Artificial Sequence: Synthetic peptide
  REGION 1..12: note = N-terminal RuvC-like domain
DLGSTSLGWAIF

SEQ ID NO: 108 - protein, 12 aa, Campylobacter jejuni
  source 1..12: note = Campylobacter jejuni, NCTC 11168
DIGISSIGWAFS

SEQ ID NO: 109 - protein, 12 aa, Parvibaculum lavamentivorans
  source 1..12: note = Parvibaculum lavamentivorans DS-1
DIGTTSIGFSVI

SEQ ID NO: 110 - protein, 12 aa, Dinoroseobacter shibae
  source 1..12: note = Dinoroseobacter shibae DFL 12
DIGTSSIGWWLY

SEQ ID NO: 111 - protein, 12 aa, Nitrobacter hamburgensis
  source 1..12: note = Nitrobacter hamburgensis X14
DLGSNSLGWFVT

SEQ ID NO: 112 - protein, 12 aa, Bradyrhizobium sp.
  source 1..12: note = Bradyrhizobium sp. BTAi1
DLGANSLGWFVV

SEQ ID NO: 113 - protein, 15 aa, synthetic construct
  REGION 1..15: note = Description of Artificial Sequence: Synthetic peptide
  REGION 1..15: note = N-terminal RuvC-like domain
DIGLRIGITSCGWSI

SEQ ID NO: 114 - protein, 12 aa, synthetic construct
  REGION 1..12: note = Description of Artificial Sequence: Synthetic peptide
  REGION 1..12: note = N-terminal RuvC-like domain
DMGAKYTGVFYA

SEQ ID NO: 115 - protein, 12 aa, Wolinella succinogenes
  source 1..12: note = Wolinella succinogenes DSM 1740
DLGGKNTGFFSF

SEQ ID NO: 116 - protein, 12 aa, synthetic construct
  REGION 1..12: note = Description of Artificial Sequence: Synthetic peptide
  REGION 1..12: note = N-terminal RuvC-like domain
DLGVKNTGVFSA

SEQ ID NO: 117 - protein, 12 aa, synthetic construct
  REGION 1..12: note = Description of Artificial Sequence: Synthetic peptide
  REGION 1..12: note = N-terminal RuvC-like domain
DLGAKFTGVALY

SEQ ID NO: 118 - protein, 12 aa, Legionella pneumophila
  source 1..12: note = Legionella pneumophila str. Paris
DLGGKFTGVCLS

SEQ ID NO: 119 - protein, 12 aa, synthetic construct
  REGION 1..12: note = Description of Artificial Sequence: Synthetic peptide
  REGION 1..12: note = N-terminal RuvC-like domain
DLGGTYTGTFIT

SEQ ID NO: 120 - protein, 12 aa, Streptococcus thermophilus
DIGTNSVGWAVT

SEQ ID NO: 121 - protein, 12 aa, Eubacterium yurii
DVGTNSVGWAVT

SEQ ID NO: 122 - protein, 12 aa, synthetic construct
  REGION 1..12: note = Description of Artificial Sequence: Synthetic peptide
  REGION 1..12: note = N-terminal RuvC-like domain
DMGTNSVGWAVT

SEQ ID NO: 123 - protein, 12 aa, Solobacterium moorei
  source 1..12: note = Solobacterium moorei F0204
DVGTSSVGWAVT

SEQ ID NO: 124 - protein, 27 aa, Treponema denticola
DIDHIYPQSKIKDDSISNRVLVCSSCN

SEQ ID NO: 125 - protein, 27 aa, Coprococcus catus
  source 1..27: note = Coprococcus catus GD-7
DIDHIYPQSKTMDDSLNNRVLVKKNYN

SEQ ID NO: 126 - protein, 27 aa, Peptoniphilus sp.
  source 1..27: note = Peptoniphilus duerdenii
DQDHIYPKSKIYDDSLENRVLVKKNLN

SEQ ID NO: 127 - protein, 27 aa, Catenibacterium mitsuokai
  source 1..27: note = Catenibacterium mitsuokai DSM 15897
QIDHIVPQSLVKDDSFDNRVLVVPSEN

SEQ ID NO: 128 - protein, 27 aa, Streptococcus mutans
DIDHIIPQAFIKDNSIDNRVLTSSKEN

SEQ ID NO: 129 - protein, 27 aa, Streptococcus thermophilus
DIDHIIPQAFLKDNSIDNKVLVSSASN

SEQ ID NO: 130 - protein, 27 aa, Oenococcus kitaharae
  source 1..27: note = Oenococcus kitaharae DSM 17330
DIDHIIPQAYTKDNSLDNRVLVSNITN

SEQ ID NO: 131 - protein, 27 aa, Listeria innocua
DIDHIVPQSFITDNSIDNLVLTSSAGN

SEQ ID NO: 132 - protein, 27 aa, Streptococcus pyogenes
DVDHIVPQSFLKDDSIDNKVLTRSDKN

SEQ ID NO: 133 - protein, 27 aa, Acidaminococcus sp.
  source 1..27: note = Acidaminococcus sp. D21
NIDHIYPQSMVKDDSLDNKVLVQSEIN

SEQ ID NO: 134 - protein, 27 aa, Lactobacillus rhamnosus
  source 1..27: note = Lactobacillus rhamnosus GG
DIDHILPQSLIKDDSLDNRVLVNATIN

SEQ ID NO: 135 - protein, 27 aa, synthetic construct
  REGION 1..27: note = Description of Artificial Sequence: Synthetic peptide
  REGION 1..27: note = HNH-LIKE domain
DIDHILPQSFIKDDSLENRVLVKKAVN

SEQ ID NO: 136 - protein, 27 aa, Staphylococcus pseudintermedius
  source 1..27: note = Staphylococcus pseudintermedius ED99
EVDHIFPRSFIKDDSIDNKVLVIKKMN

SEQ ID NO: 137 - protein, 27 aa, synthetic construct
  REGION 1..27: note = Description of Artificial Sequence: Synthetic peptide
  REGION 1..27: note = HNH-LIKE domain
EVDHIIPRSYIKDDSFENKVLVYREEN

SEQ ID NO: 138 - protein, 27 aa, Bifidobacterium bifidum
  source 1..27: note = Bifidobacterium bifidum S17
DIDHIIPQAVTQNDSIDNRVLVARAEN

SEQ ID NO: 139 - protein, 27 aa, Mycoplasma gallisepticum
  source 1..27: note = Mycoplasma gallisepticum str. F
EIDHIIPYSISFDDSSSNKLLVLAESN

SEQ ID NO: 140 - protein, 27 aa, Mycoplasma canis
  source 1..27: note = Mycoplasma canis PG 14
EIDHIIPYSLCFDDSSANKVLVHKQSN

SEQ ID NO: 141 - protein, 27 aa, Ilyobacter polytropus
  source 1..27: note = Ilyobacter polytropus DSM 2926
DIDHIIPYSRSMDDSYSNKVLVLSGEN

SEQ ID NO: 142 - protein, 27 aa, synthetic construct
  REGION 1..27: note = Description of Artificial Sequence: Synthetic peptide
  REGION 1..27: note = HNH-LIKE domain
DIDHIIPYSKSMDDSFNNKVLCLAEEN

SEQ ID NO: 143 - protein, 27 aa, Campylobacter jejuni
EIDHIYPYSRSFDDSYMNKVLVFTKQN

SEQ ID NO: 144 - protein, 27 aa, Clostridium cellulolyticum
  source 1..27: note = Clostridium cellulolyticum H10
QIDHIYPYSRSMDDSYMNKVLVLTDEN

SEQ ID NO: 145 - protein, 27 aa, synthetic construct
  REGION 1..27: note = Description of Artificial Sequence: Synthetic peptide
  REGION 1..27: note = HNH-LIKE domain
EIDHIIPFSRSFDDSLSNKILVLGSEN

SEQ ID NO: 146 - protein, 27 aa, Neisseria meningitidis
EIDHALPFSRTWDDSFNNKVLVLGSEN

SEQ ID NO: 147 - protein, 27 aa, Pasteurella multocida
  source 1..27: note = Pasteurella multocida str. Pm70
EIDHALPFSRTWDDSFNNKVLVLASEN

SEQ ID NO: 148 - protein, 27 aa, Enterococcus faecalis
  source 1..27: note = Enterococcus faecalis TX0012
EIDHIIPISISLDDSINNKVLVLSKAN

SEQ ID NO: 149 - protein, 27 aa, Eubacterium dolichum
  source 1..27: note = Eubacterium dolichum DSM 3991
EVDHIIPISISLDDSITNKVLVTHREN

SEQ ID NO: 150 - protein, 27 aa, synthetic construct
  REGION 1..27: note = Description of Artificial Sequence: Synthetic peptide
  REGION 1..27: note = HNH-LIKE domain
QVDHALPYSRSYDDSKNNKVLVLTHEN

SEQ ID NO: 151 - protein, 27 aa, Streptococcus thermophilus
  source 1..27: note = Streptococcus thermophilus LMD-9
EVDHILPLSITFDDSLANKVLVYATAN

SEQ ID NO: 152 - protein, 27 aa, Eubacterium rectale
EIDHIIPRSISFDDARSNKVLVYRSEN

SEQ ID NO: 153 - protein, 27 aa, Staphylococcus lugdunensis
  source 1..27: note = Staphylococcus lugdunensis M23590
EVDHIIPRSVSFDNSYHNKVLVKQSEN

SEQ ID NO: 154 - protein, 27 aa, synthetic construct
  REGION 1..27: note = Description of Artificial Sequence: Synthetic peptide
  REGION 1..27: note = HNH-LIKE domain
DIDHILPYSITFDDSFRNKVLVTSQEN

SEQ ID NO: 155 - protein, 27 aa, Wolinella succinogenes
  source 1..27: note = Wolinella succinogenes DSM 1740
EIDHILPRSRSADDSFANKVLCLARAN

SEQ ID NO: 156 - protein, 27 aa, unidentified
  source 1..27: note = Candidatus Puniceispirillum marinum
EIEHLLPFSLTLDDSMANKTVCFRQAN

SEQ ID NO: 157 - protein, 27 aa, Azospirillum sp.
  source 1..27: note = Azospirillum sp. B510
DIDHILPFSVSLDDSAANKVVCLREAN

SEQ ID NO: 158 - protein, 27 aa, Bradyrhizobium sp.
  source 1..27: note = Bradyrhizobium sp. BTAi1
DIDHLIPFSISWDDSAANKVVCMRYAN

SEQ ID NO: 159 - protein, 27 aa, Nitrobacter hamburgensis
  source 1..27: note = Nitrobacter hamburgensis X14
DIDHILPVAMTLDDSPANKIICMRYAN

SEQ ID NO: 160 - protein, 27 aa, synthetic construct
  REGION 1..27: note = Description of Artificial Sequence: Synthetic peptide
  REGION 1..27: note = HNH-LIKE domain
DVDHILPYSRTLDDSFPNRTLCLREAN

SEQ ID NO: 161 - protein, 27 aa, synthetic construct
  REGION 1..27: note = Description of Artificial Sequence: Synthetic peptide
  REGION 1..27: note = HNH-LIKE domain
EIEHILPFSRTLDDSLNNRTVAMRRAN

SEQ ID NO: 162 - protein, 27 aa, Lactobacillus coryniformis
  source 1..27: note = Lactobacillus coryniformis KCTC 3535
EVDHIIPYSISWDDSYTNKVLTSAKCN

SEQ ID NO: 163 - protein, 27 aa, synthetic construct
  REGION 1..27: note = Description of Artificial Sequence: Synthetic peptide
  REGION 1..27: note = HNH-LIKE domain
QVDHILPWSRFGDDSYLNKTLCTARSN

SEQ ID NO: 164 - protein, 27 aa, Ralstonia syzygii
  source 1..27: note = Ralstonia syzygii R24
QVDHILPFSKTLDDSFANKVLAQHDAN

SEQ ID NO: 165 - protein, 27 aa, Helicobacter mustelae
  source 1..27: note = Helicobacter mustelae 12198
QIDHAFPLSRSLDDSQSNKVLCLTSSN

SEQ ID NO: 166 - protein, 27 aa, Mycoplasma mobile
  source 1..27: note = Mycoplasma mobile 163K
DIDHIVPRSISFDDSFSNLVIVNKLDN

SEQ ID NO: 167 - protein, 27 aa, Mycoplasma ovipneumoniae
  source 1..27: note = Mycoplasma ovipneumoniae SC01
EIEHIIPYSMSYDNSQANKILTEKAEN

SEQ ID NO: 168 - protein, 27 aa, Mycoplasma synoviae
  source 1..27: note = Mycoplasma synoviae 53
EIDHVIPYSKSADDSWFNKLLVKKSTN

SEQ ID NO: 169 - protein, 27 aa, Aminomonas paucivorans
  source 1..27: note = Aminomonas paucivorans DSM 12260
EMDHILPYSRSLDNGWHNRVLVHGKDN

SEQ ID NO: 170 - protein, 27 aa, Ruminococcus albus
  source 1..27: note = Ruminococcus albus 8
EVDHIVPYSLILDNTINNKALVYAEEN

SEQ ID NO: 171 - protein, 27 aa, synthetic construct
  REGION 1..27: note = Description of Artificial Sequence: Synthetic peptide
  REGION 1..27: note = HNH-LIKE domain
EIEHVIPQSLYFDDSFSNKVICEAEVN

SEQ ID NO: 172 - protein, 27 aa, Bacteroides fragilis
  source 1..27: note = Bacteroides fragilis, NCTC 9343
DIEHIIPQARLFDDSFSNKTLEARSVN

SEQ ID NO: 173 - protein, 27 aa, synthetic construct
  REGION 1..27: note = Description of Artificial Sequence: Synthetic peptide
  REGION 1..27: note = HNH-LIKE domain
EIEHIVPKARVFDDSFSNKTLTFHRIN

SEQ ID NO: 174 - protein, 28 aa, Finegoldia magna
DKDHIIPQSMKKDDSIINNLVLVNKNAN

SEQ ID NO: 175 - protein, 27 aa, Parvibaculum lavamentivorans
  source 1..27: note = Parvibaculum lavamentivorans DS-1
EVEHIWPRSRSFDNSPRNKTLCRKDVN

SEQ ID NO: 176 - protein, 27 aa, synthetic construct
  REGION 1..27: note = Description of Artificial Sequence: Synthetic peptide
  REGION 1..27: note = HNH-LIKE domain
IVNHIIPYNRSFDDTYHNRVLTLTETK

SEQ ID NO: 177 - protein, 27 aa, synthetic construct
  REGION 1..27: note = Description of Artificial Sequence: Synthetic peptide
  REGION 1..27: note = HNH-LIKE domain
DMEHTIPKSISFDNSDQNLTLCESYYN

SEQ ID NO: 178 - protein, 27 aa, synthetic construct
  REGION 1..27: note = Description of Artificial Sequence: Synthetic peptide
  REGION 1..27: note = HNH-LIKE domain
DIEHTIPRSAGGDSTKMNLTLCSSRFN

SEQ ID NO: 179 - protein, 27 aa, synthetic construct
  REGION 1..27: note = Description of Artificial Sequence: Synthetic peptide
  REGION 1..27: note = HNH-LIKE domain
DIEHTIPRSISQDNSQMNKTLCSLKFN

SEQ ID NO: 180 - protein, 27 aa, Rhodospirillum rubrum
DIDHVIPLARGGRDSLDNMVLCQSDAN

SEQ ID NO: 181 - protein, 27 aa, Elusimicrobium minutum
  source 1..27: note = Elusimicrobium minutum Pei191
DIEHLFPIAESEDNGRNNLVISHSACN

SEQ ID NO: 182 - protein, 27 aa, unidentified
  source 1..27: note = Sphaerochaeta globus str. Buddy
DVDHIFPRDDTADNSYGNKVVAHRQCN

SEQ ID NO: 183 - protein, 27 aa, Nitratifractor salsuginis
  source 1..27: note = Nitratifractor salsuginis DSM 16511
DIEHIVPQSLGGLSTDYNTIVTLKSVN

SEQ ID NO: 184 - protein, 27 aa, Acidothermus cellulolyticus
  source 1..27: note = Acidothermus cellulolyticus 11B
ELDHIVPRTDGGSNRHENLAITCGACN

SEQ ID NO: 185 - protein, 28 aa, Bifidobacterium longum
  source 1..28: note = Bifidobacterium longum DJO10A
EMDHIVPRKGVGSTNTRTNFAAVCAECN

SEQ ID NO: 186 - protein, 28 aa, synthetic construct
  REGION 1..28: note = Description of Artificial Sequence: Synthetic peptide
  REGION 1..28: note = HNH-LIKE domain
EMDHIVPRKGVGSTNTRVNLAAACAACN

SEQ ID NO: 187 - protein, 28 aa, synthetic construct
  REGION 1..28: note = Description of Artificial Sequence: Synthetic peptide
  REGION 1..28: note = HNH-LIKE domain
EMDHIVPRAGQGSTNTRENLVAVCHRCN

SEQ ID NO: 188 - protein, 33 aa, synthetic construct
  REGION 1..33: note = Description of Artificial Sequence: Synthetic polypeptide
  REGION 1..33: note = HNH-LIKE domain
EIDHILPRSLIKDARGIVFNAEPNLIYASSRGN

SEQ ID NO: 189 - protein, 33 aa, synthetic construct
  REGION 1..33: note = Description of Artificial Sequence: Synthetic polypeptide
  REGION 1..33: note = HNH-LIKE domain
EIDHIIPRSLTGRTKKTVFNSEANLIYCSSKGN

SEQ ID NO: 190 - protein, 33 aa, synthetic construct
  REGION 1..33: note = Description of Artificial Sequence: Synthetic polypeptide
  REGION 1..33: note = HNH-LIKE domain
EIDHIIPRSLTLKKSESIYNSEVNLIFVSAQGN

SEQ ID NO: 191 - protein, 33 aa, Legionella pneumophila
  source 1..33: note = Legionella pneumophila str. Paris
EIDHIYPRSLSKKHFGVIFNSEVNLIYCSSQGN

SEQ ID NO: 192 - protein, 33 aa, Wolinella succinogenes
  source 1..33: note = Wolinella succinogenes DSM 1740
EIDHILPRSHTLKIYGTVFNPEGNLIYVHQKCN

SEQ ID NO: 193 - protein, 30 aa, synthetic construct
  REGION 1..30: note = Description of Artificial Sequence: Synthetic polypeptide
  REGION 1..30: note = HNH-LIKE domain
ELDHIIPRSHKKYGTLNDEANLICVTRGDN

SEQ ID NO: 194 - protein, 27 aa, Akkermansia muciniphila
ELEHIVPHSFRQSNALSSLVLTWPGVN

SEQ ID NO: 195 - protein, 27 aa, Solobacterium moorei
  source 1..27: note = Solobacterium moorei F0204
DIDHIYPRSKIKDDSITNRVLVEKDIN

SEQ ID NO: 196 - protein, 28 aa, Veillonella atypica
  source 1..28: note = Veillonella atypica ACS-134-V-Col7a
YDIDHIYPRSLTKDDSFDNLVLCERTAN

SEQ ID NO: 197 - protein, 28 aa, Fusobacterium nucleatum
DIDHIYPRSKVIKDDSFDNLVLVLKNEN

SEQ ID NO: 198 - protein, 27 aa, Filifactor alocis
DRDHIYPQSKIKDDSIDNLVLVNKTYN